SEQUENCE LISTING (12 sequences)

SEQ ID NO: 1 - protein, 117 aa, synthetic construct [sequence omitted]

SEQ ID NO: 2 - protein, 108 aa, synthetic construct [sequence omitted]

SEQ ID NO: 3 - protein, 5 aa, synthetic construct
  REGION 1..5: note = HCDR1
NYWIA

SEQ ID NO: 4 - protein, 17 aa, synthetic construct
  REGION 1..17: note = HCDR2
IIYPGDSDIRYSPSFQG

SEQ ID NO: 5 - protein, 8 aa, synthetic construct
  REGION 1..8: note = HCDR3
HDIEGFDY

SEQ ID NO: 6 - protein, 12 aa, synthetic construct
  REGION 1..12: note = LCDR1
RASQSVSSSFFA

SEQ ID NO: 7 - protein, 7 aa, synthetic construct
  REGION 1..7: note = LCDR2
GASSRAT

SEQ ID NO: 8 - protein, 9 aa, synthetic construct
  REGION 1..9: note = LCDR3
QQYDSSAIT

SEQ ID NO: 9 - protein, 107 aa, synthetic construct [sequence omitted]

SEQ ID NO: 10 - protein, 330 aa, synthetic construct
  REGION 1..330: note = Heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 447 aa, synthetic construct
  REGION 1..447: note = Heavy chain
EVQLVQSGAEVKKPGESLKISCKGSGYIFTNYWIAWVRQMPGKGLESMGIIYPGDSDIRYSPSFQGQVTISADKSITTAYLQWSSLKASDTAMYYCARHDIEGFDYWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 215 aa, synthetic construct
  REGION 1..215: note = Light chain
EIVLTQSPGTLSLSPGERATLSCRASQSVSSSFFAWYQQKPGQAPRLLIYGASSRATGIPDRLSGSGSGTDFTLTITRLEPEDFAVYYCQQYDSSAITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0122294 A1
(43) Pub. Date: Apr. 17, 2025

(54) TREATMENT OF LUPUS

(71) Applicant: ASTRAZENECA AB, Södertälje (SE)

(72) Inventors: Rajendra TUMMALA, Gaithersburg, MD (US); Eric MORAND, Victoria (AU); Gabriel ABREU, Södertälje (SE)

(21) Appl. No.: 18/698,343

(22) PCT Filed: Oct. 3, 2022
(86) PCT No.: PCT/EP2022/077438

§ 371 Date: Apr. 3, 2024

Related U.S. Application Data
(60) Provisional application No. 63/262,039, filed on Oct. 4, 2021.

(51) Int. Cl. C07K 16/28; A61K 31/573; A61K 39/00; A61K 39/395; A61P 37/06

(52) U.S. Cl. CPC C07K 16/2866; A61K 31/573; A61K 39/3955; A61P 37/06; A61K 2039/505; A61K 2039/545

(57) ABSTRACT

The disclosure relates to methods and compositions for the treatment of Systemic Lupus Erythematosus (SLE).

### 1 BACKGROUND

### 1.1 Systemic Lupus Erythematosus (SLE)

Systemic lupus erythematosus (SLE) is a chronic, multisystemic, disabling autoimmune rheumatic disease of unknown aetiology. There is substantial unmet medical need in the treatment of SLE, particularly in subjects with moderate or severe disease. Long-term prognosis remains poor for many subjects.

A significant problem associated with the treatment of SLE, is the heterogeneous clinical manifestations of SLE¹. Any organ may be affected in SLE, with the skin, joints, and kidneys being the most commonly involved²⁻⁴. Incomplete disease control leads to progressive organ damage, poor quality of life, and increased mortality, with approximately half of all patients with SLE developing organ damage within 10 years of diagnosis^{5,6}. There remains the need for a medical intervention that improves SLE disease activity across multiple systems.

Clinical manifestations of SLE include, but are not limited to, constitutional symptoms, alopecia, rashes, serositis, arthritis, nephritis, vasculitis, lymphadenopathy, splenomegaly, haemolytic anaemia, cognitive dysfunction and other nervous system involvement. Increased hospitalisations and side effects of medications including chronic oral corticosteroids (OCS) and other immunosuppressive treatments add to disease burden in SLE⁷⁻⁹.

All of the therapies currently used for the treatment of SLE have well known adverse effect profiles and there is a medical need to identify new targeted therapies, particularly agents that may reduce the requirement for corticosteroids and cytotoxic agents. There have been only 2 new treatments (belimumab and anifrolumab) for SLE approved by the US Food and Drug Administration (FDA) and European Medicines Agency (EMA) in the approximately 50 years since hydroxychloroquine was approved for use in discoid lupus and SLE. However, belimumab is not approved everywhere, and the uptake has been modest. Many agents currently used to treat SLE, such as azathioprine, cyclophosphamide, and mycophenolate mofetil/mycophenolic acid, have not been approved for the disease. Furthermore, these drugs all have well-documented safety issues and are not effective in all patients for all manifestations of lupus. Antimalarial agents (e.g. hydroxychloroquine) and corticosteroids may be used to control arthralgia, arthritis, and rashes. Other treatments include nonsteroidal anti-inflammatory drugs (NSAIDs); analgesics for fever, arthralgia, and arthritis; and topical sunscreens to minimise photosensitivity. It is often difficult to taper subjects with moderate or severe disease completely off corticosteroids, which cause long-term morbidity and may contribute to early cardiovascular mortality^{8,10}. Even small daily doses of 5 to 10 mg prednisone used long-term carry increased risks of side effects such as cataracts, osteoporosis, and coronary artery disease⁸.

### 1.2 the Challenge of Finding a Treatment for SLE

The clinical development of a new drug is a lengthy and costly process with low odds of success. For molecules that enter clinical development, less than 10% will eventually be approved by health regulatory authorities¹¹. Furthermore, the early clinical development of biotherapeutics is much lengthier than for small molecules.

Phase II trials are conducted in a small number of volunteers who have the disease of interest. They are designed to test safety, pharmacokinetics, and pharmacodynamics. A phase II trial may offer preliminary evidence of drug efficacy. However, the small number of participants and primary safety concerns within a phase II trial usually limit its power to establish efficacy. A Phase III trial is required to demonstrate the efficacy and safety of a clinical candidate. Critically, many clinical candidates that have shown promise at Phase II fail at Phase III. More than 90% of novel therapeutics entering Phase I trials fail during clinical development, primarily because of failure in efficacy or safety. The probability of success at phase III, following successful Phase II, is less than 50%¹².

The process of drug development is particularly difficult for SLE. This is because SLE is an especially complex and poorly understood disease. Not only is our understanding of the genetics of SLE rudimentary, but our insight into pathogenesis of most of the clinical manifestations are still relatively limited compared to other disease.

The complexity of SLE presents those wishing to develop new therapeutics with the problem of a patient population with extensive inhomogeneity¹³. This makes protocol design for clinical trials in SLE even more difficult, for example, as regards to the choice of inclusion criteria and primary and secondary endpoints. It is further difficult to predict the disease course in each patient. This inevitably increases the background noise that reduces the statistical power of a trial. A high placebo response rate limits the range in which the tested new drug can show an efficacy signal, making clinical trials even more difficult to conduct and interpret.

The difficulty in developing effective therapeutics for SLE leads to an even higher failure rate of therapeutics in this area in clinical trials, compared to therapeutics for other indications. The development of novel therapeutics for the treatment of SLE has thus proved extremely difficult. There are many examples of clinical candidates that showed promise at Phase II but failed to show efficacy and/or safety in subsequent Phase or Phase III trials.

### 1.3 Clinical Meaningful Endpoints in SLE Trials

BICLA and SRI(4) both evaluate clinically meaningful elements of global SLE disease activity. However, it is recognized that differences in their basic metric properties make it possible for a given patient's response to be inconsistently classified by these outcome measures. The BILAG-2004 index, on which the BICLA is anchored, grades each manifestation according to severity and the physician's intention to treat; it also captures incremental, clinically meaningful improvement or worsening. In contrast, the SLEDAI-2K, on which the SRI(4) is anchored, consists of dichotomous scoring (present or absent) of each manifestation independent of severity and assigns individual items a distinct number of points resulting in differential weights of the SLEDAI-2K elements. In order to be a BICLA responder, a patient must have at least partial improvement in all severe (BILAG-2004 A) or moderate (BILAG-2004 B) clinical manifestations affected at baseline, whereas to be an SRI(4) responder a patient needs to have complete resolution of enough manifestations affected at baseline to reduce the total SLEDAI-2K score by ≥4 points.

Furthermore, a further complication with finding new effective therapies for SLE is that Clinical and laboratory measures of SLE disease activity and damage, e.g. BICLA and SRI(4), do not assess disease and treatment-related impacts of SLE on patients' daily lives. Discordance can often exist between clinical measures of disease activity in SLE and patients' perceptions of their disease and its impact on health-related quality of life (HRQoL)¹⁴. Measuring direct treatment benefit with PROs in patients with SLE in a clinical trial setting is difficult and complex. Both poor correlation between PROs and disease activity indices and discordance between patient and physician assessments of disease activity are very well known in SLE. Multiple factors including background medications, glucocorticoid use, flares, and comorbidities can confound treatment response and significantly impact HRQoL.

### 1.4 Belimumab

Belimumab is an anti-BAFF antibody approved for the treatment of SLE patients. Belimumab remains the only new treatment for SLE approved by the US Food and Drug Administration (FDA) and the European Medicines Agency (EMA) for approximately 60 years. Belimumab is also the only biologic approved for the treatment of SLE. However, belimumab does not permit steroid sparing, as evaluated by three phase 3, multicentre, double-blind, 52-week studies in adult patients with active SLE (BLISS-52, BLISS-76 and BLISS-SC)¹⁵⁻¹⁷. In these trials, sustained steroid sparing in patients receiving belimumab (IV or SC) did not achieve statistical significance¹⁵⁻¹⁷. For example, in patients receiving >7.5 mg/day of prednisone at baseline, only 18-19% of belimumab 10 mg/kg recipients were able to reduce their prednisone dose by ≥25% to ≤7.5 mg/day for 12 weeks, compared with 12-13% of placebo recipients¹⁵. In a post-hoc analysis for the BLISS-52 and BLISS-76 data sets overall exposure to all corticosteroids actually increased on average for both the belimumab and placebo treatment groups¹⁸.

### 1.5 Type I IFN and Anifrolumab

Anifrolumab (MEDI-546) is a human immunoglobulin G1 kappa (IgG1κ) monoclonal antibody (mAb) directed against subunit 1 of the type I interferon receptor (IFNAR1). It is composed of 2 identical light chains and 2 identical heavy chains, with an overall molecular weight of approximately 148 kDa. Anifrolumab inhibits binding of type I IFN to type I interferon receptor (IFNAR) and inhibits the biologic activity of all type I IFNs.

Type I interferons (IFNs) are cytokines that have been implicated in SLE pathogenesis based on the finding of increased IFN-stimulated gene expression in most patients with SLE. In the phase 3 TULIP-2 trial of anifrolumab in patients with moderate to severe SLE, treatment response (assessed using British Isles Lupus Assessment Group [BILAG]-based Composite Lupus Assessment [BICLA]) was achieved by significantly more patients receiving anifrolumab compared with placebo at Week 52¹⁹. Similar results with this composite endpoint were observed in the phase 2 MUSE and phase 3 TULIP-1 trials^{20,21}. Importantly, composite endpoints used in SLE trials, such as BICLA and the SLE responder index (SRI), dichotomize changes in disease activity across different organ domains into a binary responder versus nonresponder result. While helpful for definitive demonstration of efficacy, this approach limits the ability to interpret treatment efficacy across the many organ domains that potentially affect patients with SLE.

In these trials, responses were assessed using both the BICLA and SRI(4) composite indices¹⁹⁻²¹. An SRI(4)-based outcome was the primary endpoint in MUSE and, given the robust outcomes, it was originally selected as the primary endpoint for both TULIP-1 and TULIP-2. BICLA, a secondary endpoint that also yielded robust outcomes in MUSE and TULIP-1, was subsequently designated the primary endpoint in TULIP-2 prior to unblinding of the TULIP-2 dataset²². In TULIP-2, anifrolumab 300 mg demonstrated a statistically significant benefit compared with placebo measured by both BICLA and SRI(4) responses at Week 52; similar results were also observed in MUSE¹⁹⁻²². In TULIP-1, the effect of anifrolumab 300 mg on BICLA response was of similar magnitude to that seen in TULIP-2 and MUSE; however, the treatment difference between anifrolumab and placebo with SRI(4) did not achieve statistical significance as seen in MUSE and TULIP-2.

### 1.6 Treat-to-Target in SLE

Treat-to-target implies a process of initiating and adjusting therapy to achieve and maintain a predefined treatment goal (clinical state, laboratory marker or combination of both)²³. Compared with other rheumatic conditions, where new targeted therapies have resulted in high rates of remission or low disease activity, the effect sizes of therapies for SLE have thus far been relatively small. Therapies that are able to be used in Treat to target (T2T) are thus needed to improve outcomes for patients with systemic lupus erythematosus.

T2T endpoints in the form of low disease activity and remission have been recently developed in SLE, including the lupus low disease activity state (LLDAS). However, in patients receiving belimumab, LLDAS at week 52 was only achieved by 10.0% of the patients in BLISS-52 and 7.1% in BLISS-76, with a greater percentage within patients who received belimumab 10 mg/kg compared with patients who received placebo in BLISS-52 (11.9% vs 6.2%; p=0.030), but not in BLISS-76 (8.3% vs 6.4%; p=0.473) 24.

### 1.7 Pain in SLE

Pain is a frequent self-reported symptom in SLE and is often one of the first symptoms of the disease²⁵. Pain in SLE maybe musculoskeletal, headache, abdominal pain, and/or pain caused by Raynaud's phenomenon²⁶.

### 1.8 Conclusion

The present invention solves one or more of the above-mentioned problems.

### 2 SUMMARY

The present invention relates to a method of treating-to-target a subject having SLE, the method comprising administering to the subject a therapeutically effective amount of a type I IFN receptor (IFNAR1) inhibitor, wherein the method treats SLE in the subject. The invention is supported inter alia by data, presented for the first time herein, demonstrating that a type I IFN receptor inhibitor (anifrolumab) can be used to achieve resolution of disease activity in a subject (e.g. a Lupus Low Disease Activity State), and can therefore be used in a treat-to-target method.

The present invention also relates to a method of treating pain in a subject in need thereof, the method comprising administering to the subject a therapeutically effective amount of a type I IFN receptor (IFNAR1) inhibitor, wherein the subject has SLE, wherein the method reduces pain in the subject. The invention is supported inter alia by data, presented for the first time herein, demonstrating that administration of a type I IFN receptor inhibitor (anifrolumab) treats pain in patients with SLE. The reduction in pain may be measured using the Numerical Rating Score (NRS)

### 3 BRIEF DESCRIPTION OF THE DRAWINGS

FIG. 1: Distribution of IFN transcript scores

FIG. 2: Glucocorticoid response and changes to dosage in patients receiving glucocorticoid≥10 mg/day at baseline in TULIP-1 and TULIP-2 (pooled data)

FIG. 2A, FIG. 2B: Patients achieving sustained oral glucocorticoid dosage reduction to (FIG. 2A)≤7.5 mg/day and (FIG. 2B)≤5 mg/day at Week 52. Error bars represent 95% CI. FIG. 2C: Oral glucocorticoid AUC through Week 52 per treatment group. Error bars represent SE. FIG. 2D: Oral glucocorticoid AUC through Week 52 for glucocorticoid responders and nonresponders. Error bars represent SE. Glucocorticoid responder defined as a glucocorticoid dosage reduction to ≤7.5 mg/day by Week 40 without a dosage increase between Week 40 and Week 52 in patients with a baseline glucocorticoid dosage≥10 mg/day. AUC, area under the curve; CI, confidence interval; LS, least squares; SE, standard error.

FIG. 3: Combined BICLA and SIR (4) response and stringent BICLA response definitions at Week 52 in patients with SLE in the MUSE, TULIP-1, and TULIP-2 trials

Response rate differences favouring anifrolumab 300 mg over placebo were observed for all 5 stringent BICLA endpoints across MUSE, TULIP-1, and TULIP-2. Rates, differences, 95% Cis and nominal P values were calculated using a stratified Cochran-Mantel-Haenszel approach (stratification factors SLEDAI-2K score at screening, Day 1 GC dose, and IFNGS test status at screening). Response for all endpoints required no trial treatment discontinuation and no use of protocol-restricted medications. BICLA response, v baseline: improvements in all BILAG-2004 organ domains (A and B scores to B/C/D and C/D respectively, no BILAG-2004 domain worsening, SRI(4) response worsening; no PGA worsening (≥0.3 points). crBICLA response criteria are defined in Table 8-1. Error bars represent standard error of the mean. *Nominal P<0.05; **nominal P<0.01; ***nominal P<0.001.

FIG. 4: Spydergram of domain scores at baseline and Week 52 in SF-36 by BICLA responders vs nonresponders in TULIP-1 and TULIP-2 (pooled data)

At Week 52, BICLA responders reported greater improvements in PCS, MCS, and all eight SF-36 domains than nonresponders (all nominal p<0.001). BICLA, British Isles Lupus Assessment Group-based Composite Lupus Assessment; LS, least squares; LupusQoL, Lupus Quality of Life; SF-36, Short Form 36 Health Survey. A/G norms, age-matched and gender-matched normative values, BI, body image; BICLA, British Isles Lupus Assessment Group-based Composite Lupus Assessment; BL, baseline; BP, bodily pain; BTO, burden to others; EH, emotional health; FACIT-F, Functional Assessment of Chronic Illness Therapy-Fatigue; FG, fatigue; GH, general health; IR, intimate relationships; LS, least squares; LupusQOL, Lupus Quality of Life; MH, mental health; MMRM, mixed model for repeated measures; PAIN, pain; PF, physical functioning; PH, physical health; PLAN, planning; RE, role emotional; RP, role physical; SF, social functioning; SF-36, Short Form 36 Health Survey; VT, vitality. Age- and gender-matched normative scores are shown for SF-36 domains. The spydergram was generated using domain raw scores (range: 0-100); US age-gender norms were matched to the study population as a benchmark comparison. A higher SF-36 or LupusQoL score indicates a more positive health state, and greater positive changes from baseline. Combined baseline mean comprises all patients (BICLA responders and nonresponders). Nominal P-values are derived from the MMRM with fixed effects outlined in the Methods section, based on the least-squares mean differences between BICLA responders and nonresponders. All domains of SF-36 and LupusQoL are nominally significant in responders versus nonresponders.

FIG. 5: Spydergram of domain scores at baseline and Week 52 in LupusQOL by BICLA responders vs nonresponders in TULIP-1 and TULIP-2 (pooled data)

At Week 52, BICLA responders reported greater improvements in all LupusQOL domains (all nominal p<0.001; intimate relationships, nominal p=0.005). BICLA, British Isles Lupus Assessment Group-based Composite Lupus Assessment; LS, least squares; LupusQoL, Lupus Quality of Life; SF-36, Short Form 36 Health Survey. A/G norms, age-matched and gender-matched normative values, BI, body image; BICLA, British Isles Lupus Assessment Group-based Composite Lupus Assessment; BL, baseline; BP, bodily pain; BTO, burden to others; EH, emotional health; FACIT-F, Functional Assessment of Chronic Illness Therapy-Fatigue; FG, fatigue; GH, general health; IR, intimate relationships; LS, least squares; LupusQOL, Lupus Quality of Life; MH, mental health; MMRM, mixed model for repeated measures; PAIN, pain; PF, physical functioning; PH, physical health; PLAN, planning; RE, role emotional; RP, role physical; SF, social functioning; SF-36, Short Form 36 Health Survey; VT, vitality. Age- and gender-matched normative scores are shown for SF-36 domains. The spydergram was generated using domain raw scores (range: 0-100); US age-gender norms were matched to the study population as a benchmark comparison. A higher SF-36 or LupusQoL score indicates a more positive health state, and greater positive changes from baseline. Combined baseline mean comprises all patients (BICLA responders and nonresponders). Nominal P-values are derived from the MMRM with fixed effects outlined in the Methods section, based on the least-squares mean differences between BICLA responders and nonresponders. All domains of SF-36 and LupusQoL are nominally significant in responders versus nonresponders.

FIG. 6: LS mean changes from baseline over time in SF-36 and LupusQoL comparative domains in BICLA responders and nonresponders in TULIP-1 and TULIP-2 (pooled data)

In comparable domains of SF-36 (physical functioning, bodily pain, vitality, and mental health) (FIG. 6A) and LupusQoL (physical health, pain, fatigue, and emotional health) (FIG. 6B), improvements from baseline reported by BICLA responders occurred at the first evaluation post treatment and were maintained to Week 52. BICLA, British Isles Lupus Assessment Group-based Composite Lupus Assessment; LS, least squares; LupusQOL, Lupus Quality of Life; SF-36, Short Form 36 Health Survey.

FIG. 7: Proportion of patients reporting improvements≥MCID at Week 52 in A) SF-36, B) LupusQOL, and C) FACIT-F by BICLA responders versus nonresponders in TULIP-1 and TULIP-2 (pooled data)

At Week 52, a greater proportion of BICLA responders compared with nonresponders reported clinically meaningful improvements≥MCID in PCS, MCS, and all eight domains of SF-36 (FIG. 7A) and all LupusQOL domains (FIG. 7B). A greater proportion of BICLA responders compared with nonresponders reported FACIT-F improvements≥ MCID at Week 52 (FIG. 7C). BICLA, British Isles Lupus Assessment Group-based Composite Lupus Assessment; LS, least squares; LupusQoL, Lupus Quality of Life; SF-36, Short Form 36 Health Survey.

FIG. 8: Proportion of patients reporting scores≥normative values at baseline and Week 52 in A) SF-36 and B) FACIT-F by BICLA responders versus nonresponders in TULIP-1 and TULIP-2 (pooled data)

The proportions of BICLA responders reporting scores≥normative values at baseline were generally higher compared with BICLA nonresponders, especially in SF-36 MCS and bodily pain, vitality, and social functioning domain scores. At Week 52, a greater proportion of BICLA responders compared with nonresponders reported scores≥normative values in PCS, MCS, and all eight domains of SF-36. BICLA, British Isles Lupus Assessment Group-based Composite Lupus Assessment; BP, bodily pain; FACIT-F, Functional Assessment of Chronic Illness Therapy-Fatigue; GH, general health; MCS, mental component summary; MH, mental health; PCS, physical component summary; PF, physical functioning; RE, role emotional; RP, role physical; SF, social functioning; SF-36, Short Form 36 Health Survey; VT, vitality. The normative values for SF-36 PCS and MCS: 50; age- and gender-matched norms for SF-36 domains: PF: 83.25, RP: 82.96, BP: 72.75, GH: 69.95, VT: 55.82, SF: 83.30, RE: 86.68, and MH: 73.34. FACIT-F normative value is 43.6. For some domains, fewer BICLA responders reported scores≥normative values at Week 52 compared with baseline.

FIG. 9: LS mean changes from baseline over time in A) SF-36 and B) LupusQoL comparative domains by treatment group in TULIP-1 and TULIP-2 (pooled data)

Over time, anifrolumab-treated patients, compared with placebo, reported numerically greater LS mean changes from baseline at first evaluation in four SF-36 domains (physical functioning, bodily pain, vitality, and mental health; Week 8) and LupusQoL comparable domains (physical health, pain, fatigue, and emotional health; Week 12). These differences were maintained to Week 52, except in the LupusQoL physical health and emotional health domains. LS, least squares; LupusQoL, Lupus Quality of Life; SF-36, Short Form 36 Health Survey.

FIG. 10: LS mean changes from baseline over time in A) SF-36 and B) LupusQoL comparative domains by treatment group in TULIP-1 and TULIP-2 (pooled data)

At Week 52, a numerically greater proportion of anifrolumab-treated patients compared with placebo reported clinically meaningful improvements≥ MCID in PCS, MCS, and all eight domains of SF-36, all LupusQoL domains, and FACIT-F. LS, least squares; LupusQoL, Lupus Quality of Life; SF-36, Short Form 36 Health Survey.

FIG. 11: Hypothesized Structural Equation Path Model of Treatment, Clinical Measures, and PROs

After baseline, treatment was hypothesized as having direct effects on the clinical measures of BICLA, BILAG-2004, and SLEDAI-2K responses, and was anticipated to result in reductions in glucocorticoid dosage. In addition, a correlation (the double-headed arrow) between clinical measures and glucocorticoid reduction was anticipated, such that if treatment results in a BICLA response and an improved SLEDAI-2K/BILAG-2004 score, this should also be associated with a reduction in glucocorticoid dosage. BILAG, British Isles Lupus Assessment Group; EQ-5D-5L, EuroQol 5 Dimension 5 Level; EQ-5D VAS, EuroQol 5 Dimensions visual analog scale; FACIT-F, Functional Assessment of Chronic Illness Therapy-Fatigue; HRQoL, health-related quality of life; LupusQOL, Lupus Quality of Life; NRS, Numerical Rating Scale; PHQ-8, 8-item Patient Health Questionnaire depression scale; PRO, patient-reported outcomes; SF-36, Short Form 36 Health Survey; Tx, treatment.

FIG. 12: Week 24 and Week 52: BICLA and Number of Glucocorticoid Tapers in TULIP-1 and TULIP-2 (Pooled Data)

BICLA, British Isles Lupus Assessment Group (BILAG)-based Composite Lupus Assessment; HRQoL, health-related quality of life; Tx, treatment. Betas larger than 1.0 are mathematically possible and not indicative of something wrong with the model. FIG. 12A presents the model for Week 24 pooled data that included BICLA response and the number of glucocorticoid dosage tapers between Weeks 8 and 24 as the clinical measures. FIG. 12B presents the model for Week 52 pooled data.

FIG. 13: Week 24 and Week 52: BICLA and Percentage Change from Baseline in Glucocorticoid Dosage in TULIP-1 and TULIP-2 (Pooled Data)

BICLA, British Isles Lupus Assessment Group (BILAG)-based Composite Lupus Assessment; CFB, change from baseline; HRQoL, health-related quality of life; Tx, treatment. Betas larger than 1.0 are mathematically possible and not indicative of something wrong with the model. FIG. 13A: Week 24; FIG. 13B: Week 52.

FIG. 14: Week 24 and Week 52: BICLA and Percentage Change from Baseline in Glucocorticoid Dosage in TULIP-1 and TULIP-2 (Pooled Data)

FIG. 14A: Week 24 and Week 52: BILAG-2004 and Number of Glucocorticoid Tapers in TULIP-1 and TULIP-2 (Pooled Data). FIG. 14B: Week 24 and Week 52: SLEDAI-2K and Number of Glucocorticoid Tapers in TULIP-1 and TULIP-2 (Pooled Data). FIG. 14C: Week 24 and Week 52: BILAG-2004 and Change from Baseline in Glucocorticoid Dosage in TULIP-1 and TULIP-2 (Pooled Data). FIG. 14D: Week 24 and Week 52: SLEDAI-2K and Change from Baseline in Glucocorticoid Dosage in TULIP-1 and TULIP-2 (Pooled Data). BILAG-2004, British Isles Lupus Assessment Group 2004; CFB, change from baseline; HRQoL, health-related quality of life; Tx, treatment. Only paths significant at P≤0.05 are shown. Coefficients are equivalent to standardized regression coefficients (Betas). Tx=Treatment arm (1=anifrolumab; 2=placebo). Betas larger than 1.0 are mathematically possible and not indicative of something wrong with the model.

FIG. 15. LLDAS response rate by randomized treatment in patients with SLE in the TULIP-1 and TULIP-2 trials

Response rates were calculated using a stratified CMH approach, with stratification factors or SLEDAI-2K at screening, day 1 glucocorticoid dosage, type I IFN gene signature at screening, and study. Nominal P values were calculated using logistic regression with the same factors as for the CMH approach. *Nominal P<0.05; **nominal P<0.01; ***nominal P<0.001. CMH, Cochran-Mantel-Haenszel; LLDAS, Lupus Low Disease Activity State; SE, standard error.

FIG. 16. Concordance between patient responder status for BICLA and SRI(4) outcomes at Week 52 in TULIP-1, TULIP-2, and (%)

In TULIP-1, the proportions of patients who were both BICLA and SRI(4) responders (“dual” responders) were 42.2% for the anifrolumab group and 27.7% for the placebo group. This treatment difference was statistically significant and was consistent with those differences observed in TULIP-2 and in MUSE. Smaller proportions of patients in each study had discordant BICLA and SRI(4) outcomes. BICLA, British Isles Lupus Assessment Group-based Composite Lupus Assessment; SRI(4), Systemic Lupus Erythematosus Responder Index of ≥4.

FIG. 17. Proportion of patients with and without sustained taper of glucocorticoids to $7.5 mg/day from Week 40 to Week 52 among patients receiving ≥10 mg/day at baseline, stratified by BICLA/SRI(4) response in TULIP-1

The proportion of patients achieving glucocorticoid taper to ≤7.5 mg/day was also lower in the placebo group than in the anifrolumab 300 mg group. All patients included in this analysis were receiving glucocorticoids (prednisone or equivalent)≥10 mg/day at baseline. BICLA− and SRI(4)− refer to nonresponders; BICLA+ and SRI(4)+ refer to responders. BICLA, British Isles Lupus Assessment Group-based Composite Lupus Assessment; GC, glucocorticoid; SRI(4), Systemic Lupus Erythematosus Responder Index of ≥4.

FIG. 18. Reasons for SRI(4) response and BICLA nonresponse at Week 52 in TULIP-1 among BICLA nonresponders/SRI(4) responders

Reasons for SRI(4) response (FIG. 18A) and BICLA nonresponse (FIG. 18B) at Week 52 in TULIP-1 among BICLA nonresponders/SRI(4) responders. In the TULIP-1 BICLA nonresponder/SRI(4) responder subgroup, the majority (22/28, 78.6%) of patients in the placebo group attained the 4-point reduction in SLEDAI-2K required for an SRI(4) response as a result of their arthritis response. BICLA− and SRI(4)− refer to nonresponders; BICLA+ and SRI(4)+ refer to responders. BICLA− and SRI(4)− refer to nonresponders; BICLA+ and SRI(4)+ refer to responders. BICLA, British Isles Lupus Assessment Group-based Composite Lupus Assessment; SRI(4), Systemic Lupus Erythematosus Responder Index of ≥4

FIG. 19. Change in active joint counts from baseline to Week 52 among patients with SLEDAI-2K arthritis response in the TULIP-1 BICLA nonresponder/SRI(4) responder subgroup

Among the 22/28 BICLA nonresponders/SRI(4) responders in the placebo group who had SLEDAI-2K arthritis responses, 11 patients (50.0%) had <6 swollen and <6 tender joints at baseline compared with 2/6 (33.3%) anifrolumab-treated patients in this subgroup Active joints were both swollen and tender. Dashed line in panel A represents the threshold of reduction to <2 active joints needed for a SLEDAI-2K arthritis response.

FIG. 20: Delivery device

Anifrolumab is administered by an injection device [1] [9] such as a prefilled syringe (PFS) (FIG. 20A) or an autoinjector (AI) (FIG. 20B).

FIG. 21. Autoinjector

The autoinjector for administering anifrolumab of the functional variant thereof in exploded view (FIG. 21A), assembled (FIG. 21B) and filled with drug substance (FIG. 21C).

FIG. 22. Accessorized pre-filled syringe

The accessorized pre-filled syringe (APFS) for anifrolumab of the functional variant thereof. The primary tube is shown in assembled form (FIG. 22A) and in exploded view (FIG. 22B). The APFS with its additional components is shown in assembled form (FIG. 22C) and in exploded view FIG. 22D).

FIG. 23. Packaging for the delivery device

### 4 DETAILED DESCRIPTION

### 4.1 Methods of Treatment

The invention relates to a method of treating-to-target a subject having SLE, the method comprising administering to the subject a therapeutically effective amount of a type I IFN receptor (IFNAR1) inhibitor, wherein the method treats SLE in the subject. The target may be a Lupus Low Disease Activity State (LLDAS) in the subject, wherein the method achieves a LLDAS in the subject. The LLDAS may be sustained for 2 weeks. The LLDAS may be sustained for 4 weeks. The LLDAS may be sustained for at least 6 weeks. The LLDAS may be sustained for at least 8 weeks. The LLDAS may be sustained for at least 12 weeks. The LLDAS may be sustained for at least 16 weeks. The LLDAS may be sustained for at least 20 weeks. The LLDAS may be sustained for at least 24 weeks. The LLDAS may be sustained for at least 28 weeks. The LLDAS may be sustained for at least 32 weeks. The LLDAS may be sustained for at least 36 weeks. The LLDAS may be sustained for at least 40 weeks. The LLDAS may be sustained for at least 44 weeks. The LLDAS may be sustained for at least 48 weeks. The LLDAS may be sustained for at least 52 weeks.

The LLDAS may be achieved by at least week 52, 48, 44, 40, 36, 32, 28, 24, 20, 16 or 12 of treatment. The LLDAS may be achieved by at least week 52 of treatment. The LLDAS may be achieved by at least week 48 of treatment. The LLDAS may be achieved by at least week 44 of treatment. The LLDAS may be achieved by at least week 36 of treatment. The LLDAS may be achieved by at least week 32 of treatment. The LLDAS may be achieved by at least week 28 of treatment. The LLDAS may be achieved by at least week 24 of treatment. The LLDAS may be achieved by at least week 20 of treatment. The LLDAS may be achieved by at least week 16 of treatment. The LLDAS may be achieved by at least week 12 of treatment. The LLDAS may be sustained for at least 2 weeks. The LLDAS may be sustained for at least 4 weeks. The LLDAS may be sustained for at least 6 weeks. The LLDAS may be sustained for at least 8 weeks. The LLDAS may be sustained for at least 12 weeks. The LLDAS may be sustained for at least 16 weeks. The LLDAS may be sustained for at least 20 weeks. The LLDAS may be sustained for at least 24 weeks. The LLDAS may be sustained for at least 28 weeks. The LLDAS may be sustained for at least 32 weeks. The LLDAS may be sustained for at least 36 weeks. The LLDAS may be sustained for at least 40 weeks. The LLDAS may be sustained for at least 44 weeks. The LLDAS may be sustained for at least 48 weeks. The LLDAS may be sustained for at least 52 weeks.

The invention also relates to a method of treating SLE in a subject in need thereof, the method comprising administering to the subject a therapeutically effective amount of a type I IFN receptor (IFNAR1) inhibitor, wherein the method resolves SLE disease activity in the subject. Resolving SLE disease activity may comprise a BILCA response. Resolving SLE disease activity may comprise a complete BILCA (crBICLA) response. The crBILCA response may be obtained by week 28 of treatment. Resolving SLE disease activity in the subject may comprise attainment of a LLDAS in the subject. The LLDAS may be achieved by at least week 52, 48, 44, 40, 36, 32, 28, 24, 20, 16 or 12 of treatment. The LLDAS may be achieved by at least week 52 of treatment. The LLDAS may be achieved by at least week 48 of treatment. The LLDAS may be achieved by at least week 44 of treatment. The LLDAS may be achieved by at least week 36 of treatment. The LLDAS may be achieved by at least week 32 of treatment. The LLDAS may be achieved by at least week 28 of treatment. The LLDAS may be achieved by at least week 24 of treatment. The LLDAS may be achieved by at least week 20 of treatment. The LLDAS may be achieved by at least week 16 of treatment. The LLDAS may be achieved by at least week 12 of treatment. Resolving SLE disease activity in the subject may comprise attainment of a LLDAS in the subject and a crBILCA response. The LLDAS may be sustained.

The invention also relates to a method of treating pain in a subject in need thereof, the method comprising administering to the subject a therapeutically effective amount of a type I IFN receptor (IFNAR1) inhibitor, wherein the subject has SLE, wherein the method reduces pain in the subject. The pain may be bodily pain. The pain may be abdominal pain, headache, and/or pain associated with the Raynaud's phenomenon.

The method or methods of the invention may reduce fatigue in the subject.

The method or methods of the invention may improve the subject's mood.

The method or methods of the invention may improve the subject's physical functioning.

The method or methods of the invention may reduce SLE disease activity in the subject. The reduction in SLE disease activity may comprise a BILCA and/or SRI(4) response.

### 4.2 IFNAR1 Inhibitor

A “type I interferon receptor inhibitor” refers to a molecule that is antagonistic for the receptor of type I interferon ligands such as interferon-α and interferon-β. Such inhibitors, subsequent to administration to a patient, preferably provide a reduction in the expression of at least 1 (preferably at least 4) pharmacodynamic (PD) marker genes selected from the group consisting of IFI6, RSAD2, IFI44, IFI44L, IFI27, MX1, IFIT1, HERC5, ISG15, LAMP3, OAS3, OAS1, EPST1, IFIT3, LY6E, OAS2, PLSCR1, SIGLECI, USP18, RTP4, and DNAPTP6. The at least 4 genes may suitably be IFI27, IFI44, IFI44L, and RSAD2. The “type I interferon receptor” is preferably interferon-α/β receptor (IFNAR).

For example, the type I interferon receptor inhibitor may be an antibody or antigen-binding fragment thereof that inhibits type I IFN activity (by inhibiting the receptor). An example of a suitable antibody or antigen-binding fragment thereof (that inhibits type I IFN activity) is an interferon-α/β receptor (IFNAR) antagonist. The type I interferon receptor inhibitor may be an antibody or antigen-binding fragment thereof that inhibits type I IFN activity. Additionally or alternatively, the type I interferon receptor inhibitor may be a small molecule inhibitor of a type I interferon receptor (e.g. for pharmacological inhibition of type I interferon receptor activity).

The IFNAR1 inhibitor may be a human monoclonal antibody specific for IFNAR1. The IFNAR1 inhibitor may be a modified IgG1 class human monoclonal antibody specific for IFNAR1.

The antibody may comprise a heavy chain variable region complementarity determining region 1 (HCDR1) comprising the amino acid sequence of SEQ ID NO: 3. The antibody may comprise a heavy chain variable region complementarity determining region 2 (HCDR2) comprising the amino acid sequence of SEQ ID NO: 4. The antibody may comprise a heavy chain variable region complementarity determining region 3 (HCDR3) comprising the amino acid sequence of SEQ ID NO: 5. The antibody may comprise a light chain variable region complementarity determining region 1 (LCDR1) comprising the amino acid sequence SEQ ID NO: The antibody may comprise a light chain variable region complementarity determining region 2 (LCDR2) comprising the amino acid sequence SEQ ID NO: 7. The antibody may comprise a light chain variable region complementarity determining region 3 (LCDR3) comprising the amino acid sequence SEQ ID NO: 8.

The antibody may comprise a human heavy chain variable region comprising the amino acid sequence of SEQ ID NO: 1. The antibody may comprise a human light chain variable region comprising the amino acid sequence of SEQ ID NO: 2. The antibody may comprise a human light chain constant region comprising the amino acid sequence of SEQ ID NO: 9. The antibody may comprise a human heavy chain constant region comprising the amino acid sequence of SEQ ID NO: 10. The antibody may comprise in the Fc region an amino acid substitution of L234F, as numbered by the EU index as set forth in Kabat and wherein said antibody exhibits reduced affinity for at least one Fc ligand compared to an unmodified antibody. The antibody may comprise a human heavy chain comprising the amino acid sequence of SEQ ID NO: 11. The antibody may comprise a human light chain comprising the amino acid sequence of SEQ ID NO: 12.

The antibody may comprise: (a) a heavy chain variable region complementarity determining region 1 (HCDR1) comprising the amino acid sequence of SEQ ID NO: 3; (b) a heavy chain of SEQ ID NO: 4; c) a heavy chain variable region complementarity determining region 3 (HCDR3) comprising the amino acid sequence of SEQ ID NO: 5; (d) a light chain variable region complementarity determining region 1 (LCDR1) comprising the amino acid sequence SEQ ID NO: 6; (b) a light chain variable region complementarity determining region 2 (LCDR2) comprising the amino acid sequence SEQ ID NO: 7; c) a light chain variable region complementarity determining region 3 (LCDR3) comprising the amino acid sequence SEQ ID NO: 8.

The antibody may comprise (a) a human heavy chain comprising the amino acid sequence of SEQ ID NO: 11; and (b) a human light chain comprising the amino acid sequence of SEQ ID NO: 12. The IFNAR1 inhibitor may be anifrolumab or a functional variant thereof.

### 4.3 Pharmaceutical Composition

The present invention also relates to a pharmaceutical composition for use in the methods of the invention. The pharmaceutical composition may comprise a IFNAR1 inhibitor. The IFNAR1 inhibitor may be a human monoclonal antibody specific for IFNAR1. The IFNAR1 inhibitor may be a modified IgG1 class human monoclonal antibody specific for IFNAR1.

The antibody may comprise a heavy chain variable region complementarity determining region 1 (HCDR1) comprising the amino acid sequence of SEQ ID NO: 3. The antibody may comprise a heavy chain variable region complementarity determining region 2 (HCDR2) comprising the amino acid sequence of SEQ ID NO: 4. The antibody may comprise a heavy chain variable region complementarity determining region 3 (HCDR3) comprising the amino acid sequence of SEQ ID NO: 5. The antibody may comprise a light chain variable region complementarity determining region 1 (LCDR1) comprising the amino acid sequence SEQ ID NO: The antibody may comprise a light chain variable region complementarity determining region 2 (LCDR2) comprising the amino acid sequence SEQ ID NO: 7. The antibody may comprise a light chain variable region complementarity determining region 3 (LCDR3) comprising the amino acid sequence SEQ ID NO: 8.

The antibody may comprise a human heavy chain variable region comprising the amino acid sequence of SEQ ID NO: 1. The antibody may comprise a human light chain variable region comprising the amino acid sequence of SEQ ID NO: 2. The antibody may comprise a human light chain constant region comprising the amino acid sequence of SEQ ID NO: 9. The antibody may comprise a human heavy chain constant region comprising the amino acid sequence of SEQ ID NO: 10. The antibody may comprise in the Fc region an amino acid substitution of L234F, as numbered by the EU index as set forth in Kabat and wherein said antibody exhibits reduced affinity for at least one Fc ligand compared to an unmodified antibody. The antibody may comprise a human heavy chain comprising the amino acid sequence of SEQ ID NO: 11. The antibody may comprise a human light chain comprising the amino acid sequence of SEQ ID NO: 12.

The antibody may comprise: (a) a heavy chain variable region complementarity determining region 1 (HCDR1) comprising the amino acid sequence of SEQ ID NO: 3; (b) a heavy chain of SEQ ID NO: 4; c) a heavy chain variable region complementarity determining region 3 (HCDR3) comprising the amino acid sequence of SEQ ID NO: 5; (d) a light chain variable region complementarity determining region 1 (LCDR1) comprising the amino acid sequence SEQ ID NO: 6; (b) a light chain variable region complementarity determining region 2 (LCDR2) comprising the amino acid sequence SEQ ID NO: 7; c) a light chain variable region complementarity determining region 3 (LCDR3) comprising the amino acid sequence SEQ ID NO: 8.

The antibody may comprise (a) a human heavy chain comprising the amino acid sequence of

SEQ ID NO: 11; and (b) a human light chain comprising the amino acid sequence of SEQ ID NO: 12. The IFNAR1 inhibitor may be anifrolumab or a functional variant thereof.

### 4.4 Device

The invention also relates to an injection device comprising the pharmaceutical composition for the use of any of the invention.

The pharmaceutical in the injection device may comprise >105 mg (i.e. more than 105 mg) and <150 mg (i.e. less than 150 mg) anifrolumab or a functional variant thereof. The pharmaceutical composition in the injection device may comprise about 120 mg anifrolumab or the functional variant thereof. The pharmaceutical composition in the injection device may comprise 120 mg anifrolumab or the functional variant thereof. The concentration of anifrolumab or the functional variant thereof in the pharmaceutical composition in the injection device may be 150 mg/ml. The volume of the pharmaceutical composition in the injection device may be at least about 0.8 ml. The volume of the pharmaceutical composition may be about 0.8 ml.

The pharmaceutical composition in the injection device may comprise about 150 to 200 mg/ml anifrolumab or the functional variant thereof, about 25 to 150 mM of lysine sale and an uncharged excipient. The pharmaceutical composition in the injection device may comprise 150 mg/ml anifrolumab or the functional variant thereof. The pharmaceutical composition in the injection device may comprise 50 mM lysine HCl. The pharmaceutical composition may comprise 130 mM trehalose dihydrate. The pharmaceutical composition in the injection device may comprise about 150 to 200 mg/ml anifrolumab or the functional variant thereof, about 25 to 150 mM of lysine sale and an uncharged excipient. The pharmaceutical composition in the injection device may comprise 150 mg/ml anifrolumab or the functional variant thereof. The pharmaceutical composition may comprise 50 mM lysine HCl. The pharmaceutical composition in the injection device may comprise 130 mM trehalose dihydrate. The pharmaceutical composition in the injection device may comprise 0.05% polysorbate 80. The pharmaceutical composition in the injection device may comprise 25 mM histidine/histidine HCl. The pharmaceutical composition in the injection device may comprise 150 mg/mL anifrolumab or the functional variant thereof, 50 mM lysine HCl, 130 mM trehalose dihydrate, 0.05% polysorbate 80 and 25 mM histidine/histidine HCl.

In another aspect the invention relates to an injection device comprising a unit dose. The unit dose may comprise >105 mg (i.e. at least 105 mg) and <150 mg (i.e. less than 150 mg) anifrolumab or a functional variant thereof. The unit dose may comprise ≤135 mg (i.e. 135 mg or less) anifrolumab or the functional variant thereof. The unit dose may comprise about 120 mg anifrolumab or the functional variant thereof. The unit dose in the injection device may comprise 120 mg anifrolumab or the functional variant thereof. The unit dose in the injection device may consist essentially of >105 mg and <150 mg anifrolumab or the functional variant thereof. The unit dose in the injection device may consist essentially of ≤135 mg anifrolumab or the functional variant thereof. The unit dose in the injection device may consist essentially of about 120 mg anifrolumab or the or the functional variant thereof. The concentration of anifrolumab or the functional variant thereof in the unit dose in the injection device may be about 150 mg/ml. The volume of the unit dose in the injection device may be less than 1 ml. The unit dose in the injection device may have a volume of about 0.5 to about 1 ml. The concentration of the unit dose may be about 0.8 ml. The volume of the unit dose may be 0.8 ml. The unit dose in the injection device may comprise a formulation of about 150 to 200 mg/ml anifrolumab or the functional variant thereof, about 25 to 150 mM of lysine sale and an uncharged excipient. The unit dose in the injection device may comprise a formulation of 150 to 200 mg/ml anifrolumab or the functional variant thereof, 25 to 150 mM of lysine sale and an uncharged excipient. The unit dose comprises a formulation of 25 mM histidine-HCL, 130 mM trehalose, and 0.05% w/v polysorbate 80. The formulation may have a pH of about 5.9.

The injection device may be a pre-filled syringe (PFS). The injection device may be an accessorized pre-filed syringe (AFPS). The injection device may be an auto-injector (AI).

### 4.5 Doses and Methods of Administration

The method may comprise administering an intravenous dose of anifrolumab or the functional variant thereof to the subject. The intravenous dose may be ≥300 mg anifrolumab or the functional variant thereof. The intravenous dose may be ≤1000 mg. The intravenous dose may be about 300 mg, about 900 mg or about 1000 mg. The intravenous dose may be administered every four weeks (Q4W).

The method may comprise administering a subcutaneous dose of anifrolumab or the functional variant thereof. The subcutaneous dose may be >105 mg and <150 mg anifrolumab or the functional variant thereof. The subcutaneous dose may be ≤135 mg anifrolumab or the functional variant thereof. The subcutaneous dose may be about 120 mg. The subcutaneous dose may be administered in a single administration step. The subcutaneous dose may be administered at intervals of 6-8 days. The subcutaneous dose may be administered once per week. The subcutaneous dose may have a volume of about 0.5 to about 1 m. The subcutaneous dose may have a volume of about 0.8 ml.

The invention also relates to a unit dose for use in the methods of the invention, wherein the unit dose comprises >105 mg and ≤150 mg anifrolumab or a functional variant thereof.

The unit dose may comprise ≤135 mg (i.e. 135 mg or less) anifrolumab or the functional variant thereof. The unit dose may comprise about 120 mg anifrolumab or the functional variant thereof. The unit dose may comprise 120 mg anifrolumab or the functional variant thereof. The unit dose may consist essentially of >105 mg and <150 mg anifrolumab or the functional variant thereof. The unit dose may consist essentially of ≤135 mg anifrolumab or the functional variant thereof. The unit dose may consist essentially of about 120 mg anifrolumab or the or the functional variant thereof. The concentration of anifrolumab or the functional variant thereof in the unit dose may be about 150 mg/ml. The volume of the unit dose may be less than 1 ml. The dose or unit dose may have a volume of about 0.5 to about 1 ml. The concentration of the unit dose may be about 0.8 ml. The volume of the unit dose may be 0.8 ml. The unit dose may comprise a formulation of about 150 to 200 mg/ml anifrolumab or the functional variant thereof, about 25 to 150 mM of lysine sale and an uncharged excipient. The unit dose may comprise a formulation of 150 to 200 mg/ml anifrolumab or the functional variant thereof, 25 to 150 mM of lysine sale and an uncharged excipient. The unit dose comprises a formulation of 25 mM histidine-HCL, 130 mM trehalose, and 0.05% w/v polysorbate 80. The formulation may have a pH of about 5.9.

Administration of the dose or unit dose may provide a plasma concentration of anifrolumab or the functional variant thereof in the patient of ≥10 μg (i.e. 10 μg or more) anifrolumab or the functional variant thereof per ml of plasma (i.e. a plasma concentration of ≥10 μg/ml). Administration of the dose or unit dose may provide a plasma concentration of anifrolumab or the functional variant thereof in the subject of about 10-100 μg/ml. Administration of the dose or unit dose may provide a plasma concentration of anifrolumab or the functional variant thereof in the subject of about 20-80 μg/ml. Administration of the dose or unit dose may provide a plasma concentration of anifrolumab or the functional variant thereof in the subject of about 30-70 μg/ml. Administration of the dose or unit dose may provide a trough concentration of anifrolumab or the functional variant thereof in the subject of ≥20 μg/ml (i.e. 20 μg/ml or more). Administration of the dose or unit dose may provide a trough concentration of anifrolumab or the functional variant thereof in the subject of ≥30 μg/ml (i.e. 30 μg/ml or more). Administration of the dose or unit dose may provide a trough concentration of anifrolumab or the functional variant thereof in the subject of ≥40 μg/ml (i.e. 40 μg/ml or more). Administration of the dose or unit dose may provide a trough concentration of anifrolumab or the functional variant thereof in the subject of about 20-100 μg/ml. Administration of the dose or unit dose may provide a trough concentration of anifrolumab or the functional variant thereof in the subject of about 30-80 μg/ml. Administration of the dose or unit dose may provide a trough concentration of anifrolumab or the functional variant thereof in the subject of about 40-70 μg/ml.

The dose or unit dose may provide a therapeutic effect in the subject that is at least equivalent to a therapeutic effect provided by administration of an intravenous dose of 300 mg anifrolumab or the functional variant thereof administered once every (Q4W). The dose or unit dose may provide a trough concentration of anifrolumab or the functional variant thereof in the subject that is greater than a trough concentration of anifrolumab or the functional variant thereof provided by administration of an intravenous dose of 300 mg anifrolumab or the functional variant thereof once every 4 weeks (Q4W). The anifrolumab or the functional variant thereof may be comprised within a pharmaceutical composition. The pharmaceutical composition may comprise about 150 to 200 mg/ml anifrolumab or the functional variant thereof, about 25 to 150 mM of lysine sale and an uncharged excipient. The pharmaceutical composition may comprise 150 mg/mL anifrolumab or the functional variant thereof. The pharmaceutical composition may comprise 50 mM lysine HCl. The pharmaceutical composition may comprise 130 mM trehalose dihydrate. The pharmaceutical composition may comprise 0.05% polysorbate 80. The pharmaceutical composition may comprise 25 mM histidine/histidine HCl. The pharmaceutical composition may comprise 150 mg/mL anifrolumab or the functional variant thereof, 50 mM lysine HCl, 130 mM trehalose dihydrate, 0.05% polysorbate 80 and 25 mM histidine/histidine HCl.

The methods of the invention may comprise administering the dose or unit dose at intervals of 6-8 days. The dose or unit dose may be administered once per week (QW). The dose or unit dose may be 120 mg anifrolumab or the functional variant thereof, wherein the method comprises administering the dose in a single administration step once per week (QW). In other words, the method comprises administering 120 mg QW of anifrolumab of the functional variant thereof. The dose or unit dose may be administered once per week for at least about 4 weeks. The dose or unit dose may be administered once per week for at least about 8 weeks. The dose or unit dose may be administered once per week for at least about 12 weeks. The dose or unit dose may be administered once per week for at least about 16 weeks. The dose or unit dose may be administered once per week for at least about 20 weeks. The dose or unit dose may be administered once per week for at least about 24 weeks. The dose or unit dose may be administered once per week for at least about 28 weeks. The dose or unit dose may be administered once per week for at least about 32 weeks. The dose or unit dose may be administered once per week for about 8 weeks. The dose or unit dose may have a volume permitted it suitable delivery in a single subcutaneous administration step. The dose or unit dose may have a volume of about 0.5 to about 1 ml. The dose or unit dose may have a volume of less than 1 ml. The dose or unit dose may have a volume of about 0.8 ml.

### 4.6 the Subject

The subject may be a human subject. The subject may be an adult. The subject may be a patient with an elevated type I IFN gene signature. The subject may be a type I interferon stimulated gene signature (IFNGS)-test high patient pre-administration with the dose or unit dose. The subject may have elevated of the genes IFI27, IFI44, IFI44L, and RSAD2 in the whole blood. The method may comprise identifying the subject as IFNGS-test high patient pre-treatment with the dose or unit dose. The method may comprise measuring the expression of the genes IFI27, IFI44, IFI44L, and RSAD2 in the whole blood of the subject. The method may comprise measuring the expression of the genes IFI27, IFI44, IFI44L, and RSAD2 in the whole blood of the subject by RT-PCR.

The subject may have moderate to severe SLE.

The subject may be a type I interferon stimulated gene signature (IFNGS)-test high patient pre-treatment. The method may comprise identifying the subject as IFNGS-test high patient pre-treatment.

### 4.7 Steroids

The method or methods of the invention may comprise administering the standard of care (SOC) to the subject. The method or methods of the invention may comprise administering a steroid to the subject. The method or method of the invention may comprise steroid sparing in the subject, wherein the dose of the steroid administered to the subject is tapered from a pre-sparing dose at baseline to a post-sparing dose.

Many patients with SLE receive corticosteroids (glucocorticoids, oral corticosteroids, OCS). However, corticosteroids are associated with organ damage. Anifrolumab permits tapering of the corticosteroids (glucocorticoids) in SLE patients (steroid sparing). The method of treatment or method may comprise administering a corticosteroid to the subject, optionally wherein the corticosteroid is an oral corticosteroid. The method may comprise tapering dose of corticosteroids administered to the subject (steroid sparing). The method may comprise administering a first dose of the corticosteroid and subsequently administering a second dose of the corticosteroid, wherein the second dose of the corticosteroid is lower than the first dose of the corticosteroid. The second dose of the corticosteroid may be about a 7.5 mg prednisone-equivalent dose or less. The second dose of the corticosteroid may be a 5 mg prednisone-equivalent dose or less. The method or method of treatment may comprise administrating the second dose of the corticosteroid once per day. The first dose of the corticosteroid may be about a 10 mg prednisone-equivalent dose. The method may comprise tapering the dose of corticosteroid administered to the patient from 10 mg or more per day to less than 10 mg per day. The method or method of treatment may comprise administering the second dose of the corticosteroid once per day. The method may permit administration of a reduced dose of corticosteroids that is sustained for weeks. The second dose of the corticosteroid may be administered for at least 24 weeks. The second dose of the corticosteroid may be administered for at least 28 weeks.

The method may comprise steroid sparing in the subject, wherein the dose of the steroid administered to the subject is tapered from a pre-sparing dose at baseline to a post-sparing dose. The post-sparing dose may be ≤7.5 mg/day prednisone or prednisone equivalent dose. The pre-sparing dose may be 20 mg/day prednisone or prednisone equivalent dose. The steroid may comprise a glucocorticoid. The steroid may comprise an oral glucocorticoid. The steroid may be selected from the group consisting of hydrocortisone, mometasone, fluticasone, fluocinolone acetonide, fluocinolone, flurandrenolone acetonide, ciclesonide, budesonide, beclomethasone, deflazacort, flunisolide, beclomethasone dipropionate, betamethasone, betamethasone valerate, methylprednisolone, dexamethasone, prednisolone, cortisol, triamcinolone, clobetasol, clobetasol propionate, clobetasol butyrate, cortisone, corticosterone, clocortolone, dihydroxycortisone, alclometasone, amcinonide, diflucortolone valerate, flucortolone, fluprednidene, fluandrenolone, fluorometholone, halcinonide, halobetasol, desonide, diflorasone, flurandrenolide, fluocinonide, prednicarbate, desoximetasone, fluprednisolone, prednisone, azelastine, dexamethasone 21-phosphate, fludrocortisone, flumethasone, fluocinonide, halopredone, hydrocortisone 17-valerate, hydrocortisone 17-butyrate, hydrocortisone 21-acetate, prednisolone, prednisolone 21-phosphate, clobetasol propionate, triamcinolone acetonide, or a mixture thereof. The steroid may be prednisone.

Many patients with SLE receive corticosteroids (glucocorticoids, oral corticosteroids, OCS). However, corticosteroids are associated with organ damage. Anifrolumab permits tapering of the corticosteroids (glucocorticoids) in SLE patients (steroid sparing). The method of treatment or method may comprise administering a corticosteroid to the subject, optionally wherein the corticosteroid is an oral corticosteroid. The method may comprise tapering dose of corticosteroids administered to the subject (steroid sparing). The method may comprise administering a first dose of the corticosteroid and subsequently administering a second dose of the corticosteroid, wherein the second dose of the corticosteroid is lower than the first dose of the corticosteroid. The second dose of the corticosteroid may be about a 7.5 mg prednisone-equivalent dose or less. The second dose of the corticosteroid may be a 5 mg prednisone-equivalent dose or less. The method or method of treatment may comprise administrating the second dose of the corticosteroid once per day. The first dose of the corticosteroid may be about a 10 mg prednisone-equivalent dose. The method may comprise tapering the dose of corticosteroid administered to the patient from 10 mg or more per day to less than 10 mg per day. The method or method of treatment may comprise administering the second dose of the corticosteroid once per day. The method may permit administration of a reduced dose of corticosteroids that is sustained for weeks. The second dose of the corticosteroid may be administered for at least 24 weeks. The second dose of the corticosteroid may be administered for at least 28 weeks.

The method may comprise steroid sparing in the subject, wherein the dose of the steroid administered to the subject is tapered from a pre-sparing dose at baseline to a post-sparing dose. The post-sparing dose may be ≤7.5 mg/day prednisone or prednisone equivalent dose. The pre-sparing dose may be 20 mg/day prednisone or prednisone equivalent dose. The steroid may comprise a glucocorticoid. The steroid may comprise an oral glucocorticoid. The steroid may be selected from the group consisting of hydrocortisone, mometasone, fluticasone, fluocinolone acetonide, fluocinolone, flurandrenolone acetonide, ciclesonide, budesonide, beclomethasone, deflazacort, flunisolide, beclomethasone dipropionate, betamethasone, betamethasone valerate, methylprednisolone, dexamethasone, prednisolone, cortisol, triamcinolone, clobetasol, clobetasol propionate, clobetasol butyrate, cortisone, corticosterone, clocortolone, dihydroxycortisone, alclometasone, amcinonide, diflucortolone valerate, flucortolone, fluprednidene, fluandrenolone, fluorometholone, halcinonide, halobetasol, desonide, diflorasone, flurandrenolide, fluocinonide, prednicarbate, desoximetasone, fluprednisolone, prednisone, azelastine, dexamethasone 21-phosphate, fludrocortisone, flumethasone, fluocinonide, halopredone, hydrocortisone 17-valerate, hydrocortisone 17-butyrate, hydrocortisone 21-acetate, prednisolone, prednisolone 21-phosphate, clobetasol propionate, triamcinolone acetonide, or a mixture thereof. The steroid may be prednisone.

### 4.8 Kit

The invention also relates to a kit comprising the injection device of the invention, and instructions for use. The instructions for use may comprise instructions for subcutaneous administration of the pharmaceutical composition. The kit may include packaging, wherein the packaging is adapted to hold the injection device and the instructions for use. The instructions for use may specify the method a method or methods of the invention. The instructions for use may specify that the pharmaceutical composition is for use in a method of treating-to-target a subject having SLE, and/or a method of treating pain in a subject in need thereof, wherein the subject has SLE.

The invention also relates to methods of manufacturing the kit of the invention.

The instructions for use may specify that the type I IFN inhibitor is approved for use in SLE patients. The instructions for use may specify that the type I IFN inhibitor is approved for use in a method or methods of the invention.

### 4.9 Formulations

The anifrolumab or the functional variant thereof may be comprised within a pharmaceutical composition. The pharmaceutical composition may comprise about 150 to 200 mg/ml anifrolumab or the functional variant thereof, about 25 to 150 mM of lysine sale and an uncharged excipient. The pharmaceutical composition may comprise 150 mg/mL anifrolumab or the functional variant thereof. The pharmaceutical composition may comprise 50 mM lysine HCl. The pharmaceutical composition may comprise 130 mM trehalose dihydrate. The pharmaceutical composition may comprise 0.05% polysorbate 80. The pharmaceutical composition may comprise 25 mM histidine/histidine HCl. The pharmaceutical composition may comprise 150 mg/mL anifrolumab or the functional variant thereof, 50 mM lysine HCl, 130 mM trehalose dihydrate, 0.05% polysorbate 80 and 25 mM histidine/histidine HCl.

Stable formulations suitable for administration to subjects and comprising anifrolumab are described in detail in U.S. Pat. No. 10,125,195 B1, which is incorporated herein in its in entirety.

### 5 DEFINITIONS

### 5.1 Type I IFN Receptor Inhibitor

A “type I interferon receptor inhibitor” refers to a molecule that is antagonistic for the receptor of type I interferon ligands such as interferon-α and interferon-β. Such inhibitors, subsequent to administration to a patient, preferably provide a reduction in the expression of at least 1 (preferably at least 4) pharmacodynamic (PD) marker genes selected from the group consisting of IFI6, RSAD2, IFI44, IFI44L, IFI27, MX1, IFIT1, HERC5, ISG15, LAMP3, OAS3, OAS1, EPST1, IFIT3, LY6E, OAS2, PLSCR1, SIGLECI, USP18, RTP4, and DNAPTP6. The at least 4 genes may suitably be IFI27, IFI44, IFI44L, and RSAD2. The “type I interferon receptor” is preferably interferon-α/B receptor (IFNAR).

For example, the type I interferon receptor inhibitor may be an antibody or antigen-binding fragment thereof that inhibits type I IFN activity (by inhibiting the receptor). An example of a suitable antibody or antigen-binding fragment thereof (that inhibits type I IFN activity) is an interferon-α/β receptor (IFNAR) antagonist.

Additionally or alternatively, the type I interferon receptor inhibitor may be a small molecule inhibitor of a type I interferon receptor (e.g. for pharmacological inhibition of type I interferon receptor activity).

The type I interferon receptor inhibitor may be an antibody or antigen-binding fragment thereof that inhibits type I IFN activity. A particularly preferred type I interferon receptor inhibitor is the antibody anifrolumab or a functional variant thereof. Anifrolumab is a monoclonal antibody targeting IFNAR1 (the receptor for a, B, and w interferons). Disclosure related to anifrolumab can be found in U.S. Pat. Nos. 7,662,381 and 9,988,459, which are incorporated herein by reference.

### 5.1.1 Anifrolumab

Anifrolumab (MEDI-546, anifro, ANI) is a human immunoglobulin G1 kappa (IgG1κ) monoclonal antibody (mAb) directed against subunit 1 of the type I interferon receptor (IFNAR1). Anifrolumab downregulates IFNAR signalling and suppresses expression of IFN-inducible genes. Disclosures related to anifrolumab can be found in U.S. Pat. Nos. 7,662,381 and 9,988,459, which are incorporated herein by reference in their entirety. Sequence information for anifrolumab is provided in Table 5-1: Sequences.

TABLE 5-1
Sequences

| | |
|---|---|
| Anifrolumab VH | EVQLVQSGAEVKKPGESLKISCKGSGYIFTNYWIAWVRQMPGKGLESMG |
| (SEQ ID NO: 1) | IIYPGDSDIRYSPSFQGQVTISADKSITTAYLQWSSLKASDTAMYYCAR |
| | HDIEGFDYWGRGTLVTVSS |
| Anifrolumab VL | EIVLTQSPGTLSLSPGERATLSCRASQSVSSSFFAWYQQKPGQAPRLLIY |
| (SEQ ID NO: 2) | GASSRATGIPDRLSGSGSGTDFTLTITRLEPEDFAVYYCQQYDSSAITFG |
| | QGTRLEIK |
| HCDR1 (SEQ ID NO: 3) | NYWIA |
| HCDR2 (SEQ ID NO: 4) | IIYPGDSDIRYSPSFQG |
| HCDR3 (SEQ ID NO: 5) | HDIEGFDY |
| LCDR1 (SEQ ID NO: 6) | RASQSVSSSFFA |
| LCDR2 (SEQ ID NO: 7) | GASSRAT |
| LCDR3 (SEQ ID NO: 8) | QQYDSSAIT |
| Light chain constant | RTVAAPSVFIFPPSDEQLKSGTASVVCLLNNFYPREAKVQWKVDNALQS |
| region | GNSQESVTEQDSKDSTYSLSSTLTLSKADYEKHKVYACEVTHQGLSSPV |
| (SEQ ID NO: 9) | TKSFNRGEC |
| Heavy chain constant | ASTKGPSVFPLAPSSKSTSGGTAALGCLVKDYFPEPVTVSWNSGALTSG |
| region | VHTFPAVLQSSGLYSLSSVVTVPSSSLGTQTYICNVNHKPSNTKVDKRVE |
| (SEQ ID NO: 10) | PKSCDKTHTCPPCPAPEFEGGPSVFLFPPKPKDTLMISRTPEVTCVVVDV |
| | SHEDPEVKFNWYVDGVEVHNAKTKPREEQYNSTYRVVSVLTVLHQDWL |
| | NGKEYKCKVSNKALPASIEKTISKAKGQPREPQVYTLPPSREEMTKNQVS |
| | LTCLVKGFYPSDIAVEWESNGQPENNYKTTPPVLDSDGSFFLYSKLTVDK |
| | SRWQQGNVFSCSVMHEALHNHYTQKSLSLSPGK |
| Heavy chain | EVQLVQSGAEVKKPGESLKISCKGSGYIFTNYWIAWVRQMPGKGLESMGII |
| (SEQ ID NO: 11) | YPGDSDIRYSPSFQGQVTISADKSITTAYLQWSSLKASD TAMYYCARHD |
| | IEGFDYWGRGTLVTVSSASTKGPSVFPLAPSSKSTSGGTAALGCLVKDYF |
| | PEPVTVSWNSGALTSGVHTFPAVLQSSGLYSLSSVVTVPS SSLGTQTYIC |
| | NVNHKPSNTKVDKRVEPKSCDKTHTCPPCPAPEFEGGPSVFLFPPKPKD |
| | TLMISRTPEVTCVVVDVSHEDPEVKFNWYVDGVEVHNAKTKPRE |
| | EQYNSTYRVVSVLTVLHQDWLNGKEYK CKVSNKALPA SIEKTISKAK |
| | GQPREPQVYTLPPSREEMTKNQVSLTCLVKGFYPSDIAVEWESNGQPEN |
| | NYKTTPPVLDSDGSFFLYSKLTVDKSRWQQGNVFSCSVMHEALHNHYT |
| | QKS LSLSPGK |
| Light chain | EIVLTQSPGTLSLSPGERATLSCRASQSVS SSFFAWYQQK PGQAPRLLI |
| (SEQ ID NO: 12) | YGASSRATGIPDRLSGSGSGT DFTLTITRLE PEDFAVYYCQ QYDSSAI |
| | TFGQGTRLEIKRTVAAPSVFIFPPSDEQLKSGT ASVVCLLNNF YPREAK |
| | VQWKVDNALQSGNSQESVTEQDSKDSTYSLSSTLTLSKADYEKHKVYACEV |
| | THQGLSSPVTKSFNRGEC |

Anifrolumab is an immunoglobulin comprising an HCDR1, HCDR2 and HCDR3 of SEQ ID NO: 3, SEQ ID NO: 4, and SEQ ID NO: 5, respectively (or functional variant thereof); and an LCDR1, LCDR2 and LCDR3 of SEQ ID NO: 6, SEQ ID NO: 7, and SEQ ID NO: 8, respectively (or functional variant thereof). Anifrolumab is an immunoglobulin comprising a VH of SEQ ID NO: 1 and a VL of SEQ ID NO: 2.

The constant region of anifrolumab has been modified such that anifrolumab exhibits reduced affinity for at least one Fc ligand compared to an unmodified antibody. Anifrolumab is a modified IgG class monoclonal antibody specific for IFNAR1 comprising in the Fc region an amino acid substitution of L234F, as numbered by the EU index as set forth in Kabat (1991, NIH Publication 91-3242, National Technical Information Service, Springfield, Va.). Anifrolumab is a modified IgG class monoclonal antibody specific for IFNAR1 comprising in the Fc region an amino acid substitution of L234F, L235E and/or P331S, as numbered by the EU index as set forth in Kabat (1991, NIH Publication 91-3242, National Technical Information Service, Springfield, Va.). Anifrolumab is an antibody comprising a light chain constant region of SEQ ID NO: 9. Anifrolumab is an antibody comprising a heavy chain constant region of SEQ ID NO: 10. Anifrolumab is an antibody comprising a light chain constant region of SEQ ID NO: 9 and a heavy chain constant region of SEQ ID NO: 10. Anifrolumab is an antibody comprising a heavy chain of SEQ ID NO: 11. Anifrolumab is an antibody comprising a light chain of SEQ ID NO: 12. Anifrolumab is an antibody comprising a heavy chain of SEQ ID NO: 11 and a light chain of SEQ ID NO: 12.

Functional variants of anifrolumab are sequence variants that perform the same function as anifrolumab. Functional variants of anifrolumab are variants that bind the same target as anifrolumab and have the same effector function as anifrolumab. Functional anifrolumab variants include antigen-binding fragments of anifrolumab and antibody and immunoglobulin derivatives of anifrolumab. Functional variants include biosimilars and interchangeable products. The terms biosimilar and interchangeable product are defined by the FDA and EMA. The term biosimilar refers to a biological product that is highly similar to an approved (e.g. FDA approved) biological product (reference product, e.g. anifrolumab) in terms of structure and has no clinically meaningful differences in terms of pharmacokinetics, safety and efficacy from the reference product. The presence of clinically meaningful differences of a biosimilar may be assessed in human pharmacokinetic (exposure) and pharmacodynamic (response) studies and an assessment of clinical immunogenicity. An interchangeable product is a biosimilar that is expected to produce the same clinical result as the reference product in any given patient.

For example, a variant of the reference (anifrolumab) antibody may comprise: a heavy chain CDR1 having at most 2 amino acid differences when compared to SEQ ID NO: 3; a heavy chain CDR2 having at most 2 amino acid differences when compared to SEQ ID NO: 4; a heavy chain CDR3 having at most 2 amino acid differences when compared to SEQ ID NO: 5; a light chain CDR1 having at most 2 amino acid differences when compared to SEQ ID NO: 6; a light chain CDR2 having at most 2 amino acid differences when compared to SEQ ID NO: 7; and a light chain CDR3 having at most 2 amino acid differences when compared to SEQ ID NO: 8; wherein the variant antibody binds to the target of anifrolumab (e.g. IFNAR) and preferably with the same affinity.

A variant of the reference (anifrolumab) antibody may comprise: a heavy chain CDR1 having at most 1 amino acid difference when compared to SEQ ID NO: 3; a heavy chain CDR2 having at most 1 amino acid difference when compared to SEQ ID NO: 4; a heavy chain CDR3 having at most 1 amino acid difference when compared to SEQ ID NO: 5; a light chain CDR1 having at most 1 amino acid differences when compared to SEQ ID NO: 6; a light chain CDR2 having at most 1 amino acid difference when compared to SEQ ID NO: 7; and a light chain CDR3 having at most 1 amino acid difference when compared to SEQ ID NO: 8; wherein the variant antibody binds to the target of anifrolumab (e.g. IFNAR) optionally with the same affinity.

A variant antibody may have at most 5, 4 or 3 amino acid differences total in the CDRs thereof when compared to a corresponding reference (anifrolumab) antibody, with the proviso that there is at most 2 (optionally at most 1) amino acid differences per CDR. A variant antibody may have at most 2 (optionally at most 1) amino acid differences total in the CDRs thereof when compared to a corresponding reference (anifrolumab) antibody, with the proviso that there is at most 2 amino acid differences per CDR. A variant antibody may have at most 2 (optionally at most 1) amino acid differences total in the CDRs thereof when compared to a corresponding reference (anifrolumab) antibody, with the proviso that there is at most 1 amino acid difference per CDR.

A variant antibody may have at most 5, 4 or 3 amino acid differences total in the framework regions thereof when compared to a corresponding reference (anifrolumab) antibody, with the proviso that there is at most 2 (optionally at most 1) amino acid differences per framework region. Optionally a variant antibody has at most 2 (optionally at most 1) amino acid differences total in the framework regions thereof when compared to a corresponding reference (anifrolumab) antibody, with the proviso that there is at most 2 amino acid differences per framework region. Optionally a variant antibody has at most 2 (optionally at most 1) amino acid differences total in the framework regions thereof when compared to a corresponding reference (anifrolumab) antibody, with the proviso that there is at most 1 amino acid difference per framework region.

A variant antibody may comprise a variable heavy chain and a variable light chain as described herein, wherein: the heavy chain has at most 14 amino acid differences (at most 2 amino acid differences in each CDR and at most 2 amino acid differences in each framework region) when compared to a heavy chain sequence herein; and the light chain has at most 14 amino acid differences (at most 2 amino acid differences in each CDR and at most 2 amino acid differences in each framework region) when compared to a light chain sequence herein; wherein the variant antibody binds to the same target antigen as the reference (anifrolumab) antibody (e.g. IFNAR) and preferably with the same affinity.

The variant heavy or light chains may be referred to as “functional equivalents” of the reference heavy or light chains. A variant antibody may comprise a variable heavy chain and a variable light chain as described herein, wherein: the heavy chain has at most 7 amino acid differences (at most 1 amino acid difference in each CDR and at most 1 amino acid difference in each framework region) when compared to a heavy chain sequence herein; and the light chain has at most 7 amino acid differences (at most 1 amino acid difference in each CDR and at most 1 amino acid difference in each framework region) when compared to a light chain sequence herein; wherein the variant antibody binds to the same target antigen as the reference (anifrolumab) antibody (e.g. IFNAR) and preferably with the same affinity.

### 5.2 Steroids

Oral corticosteroids (OCS, glucocorticoids) include prednisone, cortisone, hydrocortisone, methylprednisolone, prednisolone and triamcinolone. Examples of equivalent doses of oral prednisone are shown in Table 5-2.

TABLE 5-2
Examples of equivalent doses of oral prednisone

Oral Prednisone and

| Equivalents | Equivalent Dose | | | | | | | | | |
|---|---|---|---|---|---|---|---|---|---|---|
| Oral Prednisone | 7.5 mg | | 10 mg | | 20 mg | | 30 mg | | 40 mg | |
| Cortisone | 37.5 | mg | 50 | mg | 100 | mg | 150 | mg | 200 | mg |
| Hydrocortisone | 30 | mg | 40 | mg | 80 | mg | 120 | mg | 160 | mg |
| Methylprednisolone | 6 | mg | 8 | mg | 16 | mg | 24 | mg | 32 | mg |
| Prednisolone | 7.5 | mg | 10 | mg | 20 | mg | 30 | mg | 40 | mg |
| Triamcinolone | 6 | mg | 8 | mg | 16 | mg | 24 | mg | 32 | mg |

### 5.3 Clinical Trials

### 5.3.1 Phase 2/Phase II/Pivotal Studies

Phase II studies gather preliminary data on effectiveness. In Phase 2 studies, researchers administer the drug to a group of patients with the disease or condition for which the drug is being developed. Typically involving a few hundred patients, these studies aren't large enough to show whether the drug will be beneficial. Instead, Phase 2 studies provide researchers with additional safety data. Researchers use these data to refine research questions, develop research methods, and design new Phase 3 research protocols.

### 5.3.2 Phase 3/Phase III/Pivotal Studies or Trials

Researchers design Phase 3 studies to demonstrate whether or not a product offers a treatment benefit to a specific population. Sometimes known as pivotal studies, these studies involve 300 to 3,000 participants. Phase 3 studies provide most of the safety data. In previous studies, it is possible that less common side effects might have gone undetected. Because these studies are larger and longer in duration, the results are more likely to show long-term or rare side effects. Regulatory bodies such as the EMA and FDA usually require a phase III clinical trial demonstrating that the product is safe and at least as effective (if not better) than available medications, before approving a new medication. Phase III clinical trials usually fail, even if they follow a successful a phase II clinical trial.

### 5.4 Dosage Forms

A unit dose (also referred to as a unit dose form, a pharmaceutical unit dose or a pharmaceutical unit dose form) is a dose formed from a single unit. A unit dose (unit dose form) is suitable for administration to a subject in a single administration step. A unit dose (unit dose form) may be packaged in a single-unit container, for example a single-use pre-filled syringe or autoinjector. Unit doses provide the advantage that they can be ordered, packaged, handled and administered as single dose units containing a pre-determined amount of a drug. Unit doses decrease administration errors and reduce waste.

### 5.5 PK/PD

Plasma levels obtainable by SC administration and IV administration may be compared on the basis of a plasma drug concentration-time curve (AUC), which reflects the body exposure to the antibody after administration of a dose of the drug. For example, during a clinical study, the patient's plasma drug concentration-time profile can be plotted by measuring the plasma concentration at several time points. Where an in silico modelling approach is employed, plasma drug concentration-time for any given dose may be predicted. The AUC (area under the curve) can then be calculated by integration of the plasma drug concentration-time curve. Suitable methodology is described in Tummala et. al.²⁷ which is incorporated herein by reference in its entirety. In the Examples described herein, PK parameters were calculated by non-compartmental analysis with Phoenix WinNonlin V/6.2 (Certara, Inc., Princeton, New Jersey, USA) and included the area under the serum concentration-time curve (AUC), clearance (CL, CL/F), maximum serum concentration (Cₘₐₓ) and time to reach maximum serum concentration (tₘₐₓ). All data were analysed with SAS System V.9.2 (SAS Institute, Inc., Cary, NC, USA).

Conveniently, a ratio of the AUC obtainable with SC administration to the AUC obtainable by IV administration (AUC_{SC}/AUC_{IV}) may be calculated, providing a numerical comparison of bioavailability provided by the dosage routes. Reference to the “AUC Ratio” herein means the AUC_{SC}/AUC_{IV} ratio. To provide statistical robustness, the AUC ratio is preferably a mean, median or mode (for example, a mean) value calculated from a plurality of repeat experiments (or computational simulations). This approach is demonstrated with reference to the Examples. The mean, median or mode (preferably mean) may be derived by pooling data obtained from multiple patients (or multiple computational simulations). Thus, the AUC Ratio may reflect the mean, median or mode (preferably mean) AUC in multiple patients.

### 5.6 Pharmacokinetics Glossary

Area under the curve (AUC): Area under the plasma drug concentration versus time curve, which serves as a measure of drug exposure.

Cₐᵥₑ: Steady-state average concentration.

Cₘₐₓ: The maximum (or peak) concentration of the drug in the plasma.

Cₘᵢₙ: Minimum plasma drug concentration.

C_{trough}: the concentration of drug in plasma at steady state immediately prior to the administration of a next dose. Trough plasma concentration (measured concentration at the end of a dosing interval at steady state [taken directly before next administration]).

LLOQ: The lower limit of quantitation, the lowest amount of an analyte in a sample that can be quantitatively determined with suitable precision and accuracy.

Linear pharmacokinetics: When the concentration of the drug in the blood or plasma increases proportionally with the increasing dose, and the rate of elimination is proportional to the concentration, the drug is said to exhibit linear pharmacokinetics. The clearance and volume of distribution of these drugs are dose-independent.

Nonlinear pharmacokinetics: As opposed to linear pharmacokinetics, the concentration of the drug in the blood or plasma does not increase proportionally with the increasing dose. The clearance and volume of distribution of these may vary depending on the administered dose. Nonlinearity may be associated with any component of the absorption, distribution, and/or elimination processes.

### 5.7 Delivery Device

As well as providing for subcutaneous administration of the antibody, the ability to self-administer (e.g. for home use) may further be enhanced by subcutaneous administration via an accessorized pre-filled syringe (APFS), an autoinjector (AI), or a combination thereof. Such devices have been found to be well-tolerated and reliable for administering subcutaneous doses of an antibody and provide further options for optimizing patient care. Indeed, such devices may reduce the burden of frequent clinic visits for patients. An example of a suitable APFS device is described in Ferguson et. al.²⁸, which is incorporated herein by reference in its entirety.

The dose elucidated by the inventors provides yet advantages in the context of APFS-administration, as an APFS device typically administers a maximal volume of 1 ml. A dose in the range of >105 mg to <155 mg can be readily accommodated by a volume of ˜0.8 ml, such that the dose(s) of the present invention are uniquely suited to APFS and AI administration. For comparison, due to viscosity of the anifrolumab, larger doses (particularly doses of >150 mg) would need to be administered within a volume of >1 ml, requiring at least two SC injections, which is inconvenient for the patient, and would require a plurality of pre-filled devices.

The delivery device may be single use, disposable system that is designed to enable manual, SC administration of the dose.

### 5.8 End Points

### 5.8.1 BILAG-2004 (British Isles Lupus Assessment Group-2004)

The BILAG-2004 is a translational index with 9 organ systems (General, Mucocutaneous, Neuropsychiatric, Musculoskeletal, Cardiorespiratory, Gastrointestinal, Ophthalmic, Renal and Haematology) that is able to capture changing severity of clinical manifestations. It has ordinal scales by design and does not have a global score; rather it records disease activity across the different organ systems at a glance by comparing the immediate past 4 weeks to the 4 weeks preceding them. It is based on the principle of physicians' intention to treat and categorises disease activity into 5 different levels from A to E:
Grade A represents very active disease requiring immunosuppressive drugs and/or a prednisone dose of >20 mg/day or equivalent.
Grade B represents moderate disease activity requiring a lower dose of corticosteroids, topical steroids, topical immunosuppressives, antimalarials, or NSAIDs
Grade C indicates mild stable disease.
Grade D implies no disease activity but the system has previously been affected
Grade E indicates no current or previous disease activity

Although the BILAG-2004 was developed based on the principle of intention to treat, the treatment has no bearing on the scoring index. Only the presence of active manifestations influences the scoring.

BILAG-defined improvement in mucocutaneous or musculoskeletal organ systems were representative of rash or arthritis, respectively.

### 5.8.2 BICLA (BILAG-Based Composite Lupus Assessment)

BICLA is a composite index that was originally derived by expert consensus of disease activity indices. BICLA response is defined as (1) at least one gradation of improvement in baseline BILAG scores in all body systems with moderate or severe disease activity at entry (e.g., all A (severe disease) scores falling to B (moderate), C (mild), or D (no activity) and all B scores falling to C or D); (2) no new BILAG A or more than one new BILAG B scores; (3) no worsening of total SLEDAI score from baseline; (4) no significant deterioration (≤10%) in physicians global assessment; and (5) no treatment failure (initiation of non-protocol treatment).

Particularly, a subject is a BICLA responder if the following criteria are met:
a) Reduction of all baseline BILAG-2004 A to B/C/D and baseline BILAG-2004 B to C/D, and no BILAG-2004 worsening in other organ systems, as defined by 1 new BILAG-2004 A or more than 1 new BILAG-2004 B item;
b) No worsening from baseline in SLEDAI-2K as defined as an increase from baseline of >0 points in SLEDAI-2K;
c) No worsening from baseline in the subjects' lupus disease activity defined by an increase ≥0.30 points on a 3-point PGA VAS;
d) No discontinuation of investigational product or use of restricted medications beyond the protocol-allowed threshold before assessment

A complete resolution (crBICLA, also referred to as a modified BICLA (mBICLA)) response requires a complete resolution of all baseline BILAG-2004 activity (all baseline A/B scores to D; no worsening of C or D scores).

### 5.8.3 CLASI (Cutaneous Lupus Erythematosus Disease Area and Severity Index Inflammatory Disease Activity)

The Cutaneous Lupus Erythematosus Disease Area and Severity Index (CLASI) was developed in 2005 as a means of specifically tracking cutaneous activity and damage in patients with CLE²⁹. The CLASI is a simple, single-page tool that separately quantifies skin disease activity and damage in each part of the body³⁰. The CLASI features a skin activity summary score (CLASI-A) and damage summary score (CLASI-D).

### 5.8.4 Lupus Low Disease Activity State (LLDAS)

The Lupus Low Disease Activity State was developed in response to the need for a definition of a low disease activity state in SLE that enabled treat-to-target strategies to be tested in this disease³¹

### 5.8.5 SRI (Systemic Lupus Erythematosus Responder Index of ≥4)

A subject achieves SRI(4) if all of the following criteria are met:
Reduction from baseline of ≥4 points in the SLEDAI-2K;
No new organ system affected as defined by 1 or more BILAG-2004 A or 2 or more
BILAG-2004 B items compared to baseline using BILAG-2004;
No worsening from baseline in the subjects' lupus disease activity defined by an increase ≥0.30 points on a 3-point PGA VAS.

SRI(X) (X=5, 6, 7, or 8) is defined by the proportion of subjects who meet the following criteria:
Reduction from baseline of ≥X points in the SLEDAI-2K;
No new organ systems affected as defined by 1 or more BILAG-2004 A or 2 or
more BILAG-2004 B items compared to baseline using BILAG-2004;
No worsening from baseline in the subjects' lupus disease activity defined by an
increase ≥0.30 points on a 3-point PGA VAS

### 5.8.6 Patient Reported Outcomes (PROs)

### 5.8.6.1 Short Form 36 Health Survey (SF-36) Version 2 (Acute)

The SF-36-v2 (acute) is a multipurpose, 36-item survey that measures 8 domains of health: physical functioning, role limitations due to physical health, bodily pain, general health perceptions, vitality, social functioning, role limitations due to emotional problems, and mental health. It yields scale scores for each of these 8 health domains, and summary measures of physical and mental health: the Physical Component Summary and Mental Component Summary.

### 5.8.6.2 Functional Assessment of Chronic Illness Therapy-Fatigue (FACIT-F)

The FACIT-F is a 13-item subject-completed questionnaire to assess the impact of fatigue over the previous 7 days. The responses range from 0 (Not at All) to 4 (Very Much). Final scores are the sum of the responses and range from 0 to 52; higher scores indicate better QoL³². Changes in scores>3 points are considered to be clinically meaningful.

### 5.8.6.3 EuroQol 5 Dimension 5 Level (EQ-5D-5L)

The EQ-5D-5L is comprised of the following 5 dimensions: mobility, self-care, usual activities, pain/discomfort and anxiety/depression³³. Each dimension has 5 response options (no problems, slight problems, moderate problems, severe problems and unable to/extreme problems) that reflect increasing levels of difficulty. EQ-5D-5L health states will be converted into a single index value using values sets from the EQ-5D-5L Crosswalk Project. The questionnaire also includes a VAS (EQ-5D VAS), where the subjects are asked to rate their health on a scale of 0-100, with 0 being worst imaginable health state and 100 being best imaginable health state.

### 5.8.6.4 Lupus Quality of Life (LupusQoL)

LupusQoL is a lupus-specific HRQoL questionnaire developed by McElhone et al, 2007³⁴ and was further validated by Jolly et al, 2010³⁵. The assessment consists of 34 items grouped in eight domains: physical health, pain, planning, intimate relationships, burden to others, emotional health, body image and fatigue. Item responses are captured using a 5-point verbal response scale (0-4; all of the time to never; and a not applicable response which is not scored). Mean raw domain is calculated as the sum of item responses divided by the number of items in the domain. The mean raw domain is then divided by 4 and multiplied by 100 for a transformed score ranging from 0 (worst HRQoL) to 100 (best HRQoL).

### 5.8.6.5 Patient Health Questionnaire Depression Scale (PHQ-8)

The PHQ-8 consists of 8 of the 9 criteria on which the DSM-IV diagnosis of depressive disorders is based³⁶. It assesses symptoms of depression over the last 2 weeks.

### 5.8.6.6 Pain Numerical Rating Scale (NRS)

In a Numerical Rating Scale (NRS), patients are asked to circle the number between 0 and 10, 0 and 20 or 0 and 100 that fits best to their pain intensity³⁷. Zero usually represents ‘no pain at all’ whereas the upper limit represents ‘the worst pain ever possible’.

### 5.8.7 SLEDAI-2K (Systemic Lupus Erythematosus Disease Activity Index 2000)

The SLEDAI-2K disease activity index consists of a list of organ manifestations, each with a definition. A certified Investigator or designated physician will complete the SLEDAI-2K assessment and decide whether each manifestation is “present” or “absent” in the last 4 weeks. The assessment also includes the collection of blood and urine for assessment of the laboratory categories of the SLEDAI-2K.

The SLEDAI-2K assessment consists of 24 lupus-related items. It is a weighted instrument, in which descriptors are multiplied by a particular organ's “weight”. For example, renal descriptors are multiplied by 4 and central nervous descriptors by 8 and these weighted organ manifestations are totalled into the final score. The SLEDAI-2K score range is 0 to 105 points with 0 indicating inactive disease. The SLEDAI-2K scores are valid, reliable, and sensitive clinical assessments of lupus disease activity. The SLEDAI-2K calculated using a timeframe of 30 days prior to a visit for clinical and laboratory values has been shown to be similar to the SLEDAI-2K with a 10-day window³⁸.

SLEDAI-2K-defined resolution of rash is defined as a score of 0 at Week 52 for those with a score≥2 for rash at baseline.

### 5.9 Type I IFN Gene Signature (IFNGS)

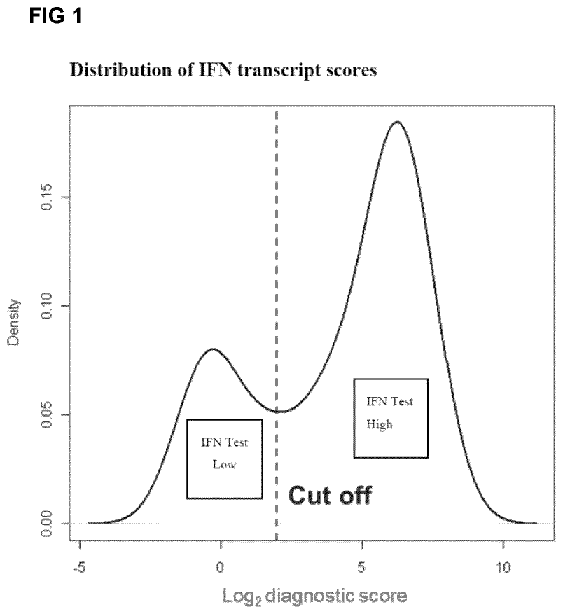

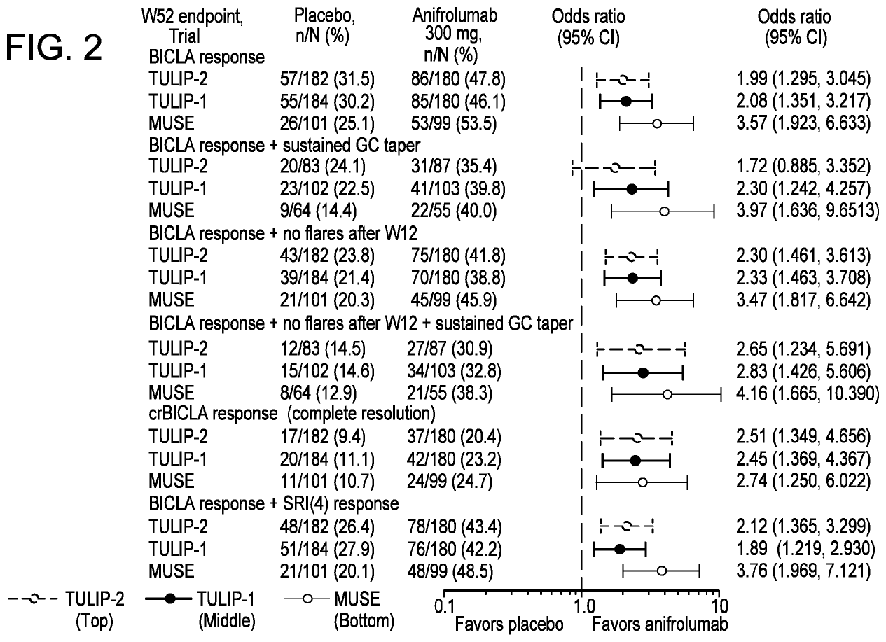

Type I IFN is considered to play a central role SLE disease pathogenesis and inhibition of this pathway is targeted by anifrolumab. To understand the relationship between type I IFN expression and response to anti-IFN therapy, it is necessary to know if a subject's disease is driven by type I IFN activation. However, direct measurement of type I IFN remains a challenge. As such, a transcript-based marker was developed to evaluate the effect of over expression of the target protein on a specific set of mRNA markers. The expression of these markers is easily detected in whole blood and demonstrates a correlation with expression in diseased tissue such as skin in SLE. The bimodal distribution of the transcript scores for SLE subjects supports defining an IFN test high and low subpopulation (FIG. 1). The type I IFN test is described in WO2011028933 A1, which is incorporated herein by reference in its entirety. The type I IFN gene signature may be used to identify a subject has a type I IFN gene signature (IFNGS)-test high patient or an IFNGS-test low patient. The IFNGS test measures expression of the genes IFI27, IFI44, IFI44L, and RSAD2 compared with 3 reference genes; 18S, ACTB and GAPDH in the whole blood of the subject. The result of the test is a score that is compared with a pre-established cut-off that classifies patients into 2 groups with low or high levels of IFN inducible gene expression (FIG. 1).

The expression of the genes may be measured by RT-PCR. Suitable primers and probes for detection of the genes may be found in WO2011028933. A suitable kit for measuring gene expression for the IFNGS test is the QIAGEN Therascreen® IFIGx RGQ RT-PCR kit (IFIGx kit), as described in Brohawn et al.³⁹, which is incorporated herein by reference in its entirety.

### 6 EXAMPLE 1: MUSE, CLINICALTRIAL.GOV IDENTIFIER: NCT01438489

MUSE was a Phase 2, multinational, multicentre, randomized, double-blind, placebo controlled, parallel-group study to evaluate the efficacy and safety of 2 intravenous (IV) treatment regimens in adult participants with chronic, moderately-to-severely active SLE with an inadequate response to standard of care (SOC) SLE. The investigational product (anifrolumab or placebo) was administered as a fixed dose every 4 weeks (28 days) for a total of 13 doses.

MUSE is described in further detail in Furie et al. 2017²⁰, which is incorporated herein by reference in its entirety.

### 7 EXAMPLE 2: TULIP I AND II, CLINICALTRIAL.GOV IDENTIFIERS: NCT02446912 AND NCT02446899

TULIP I and TULIP II were Phase 3, multicentre, multinational, randomised, double-blind, placebo-controlled studies to evaluate the efficacy and safety of an intravenous (IV) treatment regimen of two doses of anifrolumab versus placebo in subjects with moderately to severely active, autoantibody-positive systemic lupus erythematosus (SLE) while receiving standard of care (SOC) treatment.

### 7.1.1 Restricted Medications

If a subject received 1 of the following, the subject was considered a non-responder. Sulfasalazine; Danazol; Dapsone; Azathioprine>200 mg/day or at a daily dose greater than that at Week 0 (Day 1); Mycophenolate mofetil>2.0 g/day or mycophenolic acid>1.44 g/day or at a daily; dose greater than that at Week 0 (Day 1); Oral, SC, or intramuscular methotrexate>25 mg/week or at a daily dose greater than that at Week 0 (Day 1); Mizoribine>150 mg/day or at a daily dose greater than that at Week 0 (Day 1); Any change in route of administration of oral, SC, or intramuscular methotrexate; Intravenous corticosteroids>40 mg/day but≤1 gm/day methylprednisolone or equivalent; Intramuscular corticosteroids>80 mg/day methylprednisolone or equivalent; Subcutaneous or intramuscular corticosteroid precursors; Treatment with OCS>40 mg/day prednisone or equivalent; Treatment with OCS above Day 1 dose for a dosing period>14 days; Corticosteroids with a long biologic half-life (e.g., dexamethasone, betamethasone); Other immunosuppressants including but not limited to calcineurin inhibitors (e.g., cyclosporine, tacrolimus [including topical]) or leflunomide. Cyclosporine eye drops were acceptable for use in the study.

TULIP I is described in further detail in Furie et al. 2019²¹, which is incorporated herein by reference in its entirety. The results of TULIP II are presented in Morand et al. 2020¹⁹, herein incorporated by reference in its entirety.

### 8 EXAMPLE 3: NOVEL STRINGENT OUTCOME MEASURES APPLIED TO THE PHASE 2 AND 3 ANIFROLUMAB TRIALS

### 8.1 Background

Treatment of patients with systemic lupus erythematosus (SLE) should aim to lower disease activity and prevent flares, maintained with the lowest possible dose of glucocorticoids (GC). The British Isles Lupus Assessment Group (BILAG)-based Composite Lupus Assessment (BICLA) is an assessment of global disease activity that is frequently evaluated in SLE clinical trials. A BICLA response requires improvement in all domains affected at baseline, assessed by BILAG-2004, no worsening of other BILAG-2004 domains, and no worsening vs baseline of both SLE Disease Activity Index 2000 (SLEDAI-2K) and Physician's Global Assessment (PGA).

Patients with systemic lupus erythematosus (SLE) who received anifrolumab, a type I interferon receptor antibody, had greater BILAG-based Composite Lupus Assessment (BICLA) response rates vs placebo at Week (W) 52 in the phase 2 MUSE and the phase 3 TULIP-1 and TULIP-2 trials. Patients receiving anifrolumab also had fewer flares, and more patients were able to taper glucocorticoids (GC) vs placebo.

### 8.2 Objectives

To evaluate anifrolumab treatment response vs placebo in patients with SLE from TULIP-2, TULIP-1, and MUSE using more stringent BICLA definitions, as well as a novel endpoint that requires dual BICLA and SLE Responder Index (SRI[4]) responses.

### 8.3 Methods

MUSE, TULIP-1, and TULIP-2 were randomized, placebo-controlled, 52-week trials of intravenous anifrolumab (every 4 weeks for 48 weeks) in patients with moderate to severe SLE despite standard therapy For patients receiving GC≥10 mg/day at baseline, taper to ≤7.5 mg/day was considered sustained if achieved by W40 and sustained through W52. For patients receiving GC<10 mg/day at baseline, GC taper was sustained if the W40 dose was less than or equal to the baseline dose, with no increase from W40-W52. In this post hoc analysis, response rates for 5 novel endpoints were compared between anifrolumab 300 mg vs placebo groups for patients who: 1) met both BICLA and SRI(4) response criteria; 2) attained a W52 BICLA response with sustained GC taper; 3) attained a W52 BICLA response and no flares after W12 (flare defined as ≥1 new BILAG-2004 A or ≥2 new BILAG-2004 B scores vs the prior visit); 4) attained a W52 BICLA response with sustained GC taper and no flares after W12; and 5) attained a modified BICLA (mBICLA, crBICLA) response at W52 that required complete resolution of all baseline BILAG-2004 activity (all baseline A/B scores to D; no worsening of C or D scores).

### 8.3.1 Novel Stringent Outcomes Measures

In this post hoc analysis, response rates for 5 novel endpoints were compared between anifrolumab 300 mg vs placebo groups for patients who: 1) met both BICLA and SRI(4) response criteria; 2) attained a W52 BICLA response with sustained GC taper; 3) attained a W52 BICLA response and no flares after W12 (flare defined as ≥1 new BILAG-2004 A or ≥2 new BILAG-2004 B scores vs the prior visit); 4) attained a W52 BICLA response with sustained GC taper and no flares after W12; and 5) attained a modified BICLA (crBICLA) response at W52 that required complete resolution of all baseline BILAG-2004 activity (all baseline A/B scores to D; no worsening of C or D scores) (Table 8-1).

TABLE 8-1
Novel stringent outcome measures applied to data from the TULIP-2, TULIP-1, and MUSE trials

| Endpoint | Definition |
|---|---|
| 1) BICLA response at Week 52 | BICLA response^{a} + GC taper achieved by Week 40 and sustained |
| With sustained GC taper | through Week 52 (GC taper defined as a dosage of ≤7.5 mg/day in |
| | patients receiving ≥10 mg/day prednisone or equivalent at |
| | baseline) |
| 2) BICLA response at Week 52 and | BICLA response^{a} + no flares after Week 12, where flare was |
| no flares | defined as ≥1 new BILAG-2004 A or ≥2 new BILAG-2004 B scores |
| | vs the prior visit |
| 3) BICLA response at Week 52 with | BICLA response^{a} + GC taper achieved by Week 40 and sustained |
| sustained GC taper and no flares | through Week 52 (as above) + no flares after Week 12 (as above) |
| 4) crBICLA (BICLA requiring | BICLA response^{a} modified to require resolution of all baseline |
| complete resolution) response at | BILAG-2004 A/B scores to D and no worsening of C or D scores |
| Week 52 and by treatment visit | |
| 5) Dual BICLA and SRI(4) response | Met both BICLA response^{a} and SRI(4) response^{b} criteria (meaning |
| at Week 52 | patients had improvement in all organ systems with involvement at |
| | baseline and complete resolution of enough disease manifestations |
| | to achieve a ≥4-point improvement in SLEDAI-2K score) |

BICLA, British Isles Lupus Assessment Group-based Composite Lupus Assessment; BILAG, British Isles Lupus Assessment Group; crBICLA, complete-resolution BICLA; GC, glucocorticoid; PGA, Physician's Global Assessment; SLEDAI-2K, Systemic Lupus Erythematosus Disease Activity Index 2000; SRI(4), Systemic Lupus Erythematosus Responder Index of ≥4.
^{a}BICLA response² defined as all of the following: reduction of all baseline BILAG-2004 A and B domain scores to B/C/D and C/D, respectively, and no worsening in other BILAG-2004 organ systems; no increase in SLEDAI-2K score (from baseline); no increase in PGA score (≥0.3 points from baseline); no discontinuation of investigational product (IP); and no use of restricted medications beyond protocol-allowed thresholds.
^{b}SRI(4) response⁷ defined as all of the following: ≥4-point reduction in SLEDAI-2K; <1 new BILAG-2004 A or <2 new BILAG-2004 B scores; <0.3-point increase in PGA from baseline; no use of restricted medications beyond protocol-allowed thresholds; and no discontinuation of IP.

### 8.3.2 Statistical Analysis

Response rates, treatment differences, 95% confidence intervals (CIs), odds ratios, standard errors, and nominal P values were calculated using a stratified Cochran-Mantel-Haenszel approach6 (stratification factors: SLEDAI-2K score at screening, Day 1 GC dosage, and interferon gene signature [IFNGS] status at screening).

### 8.4 Results:

Evaluated patients received anifrolumab 300 mg (MUSE, n=99; TULIP-1 and TULIP-2, n=180) or placebo (MUSE, n=102; TULIP-1, n=184; TULIP-2, n=182). Demographics and baseline disease characteristics were generally balanced (Table 8-2).

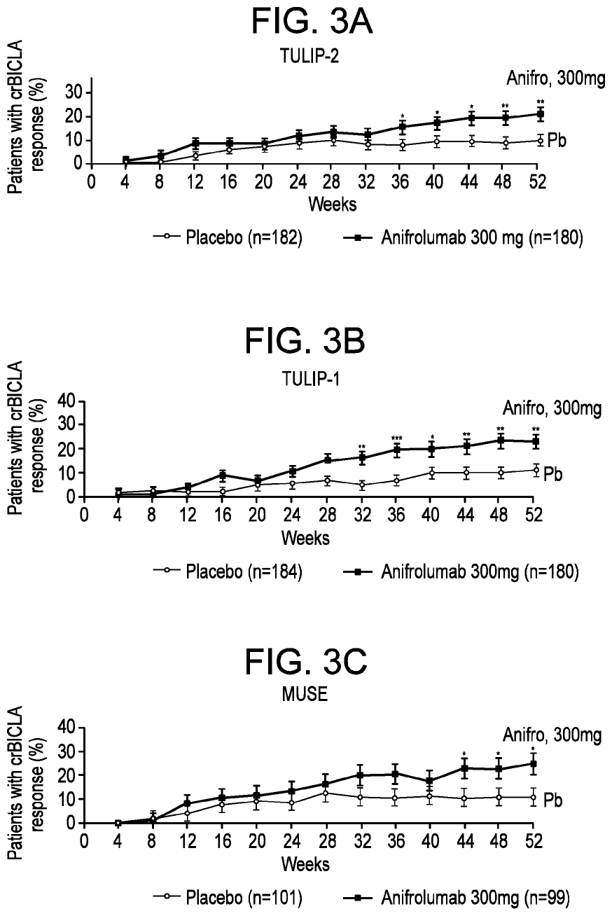

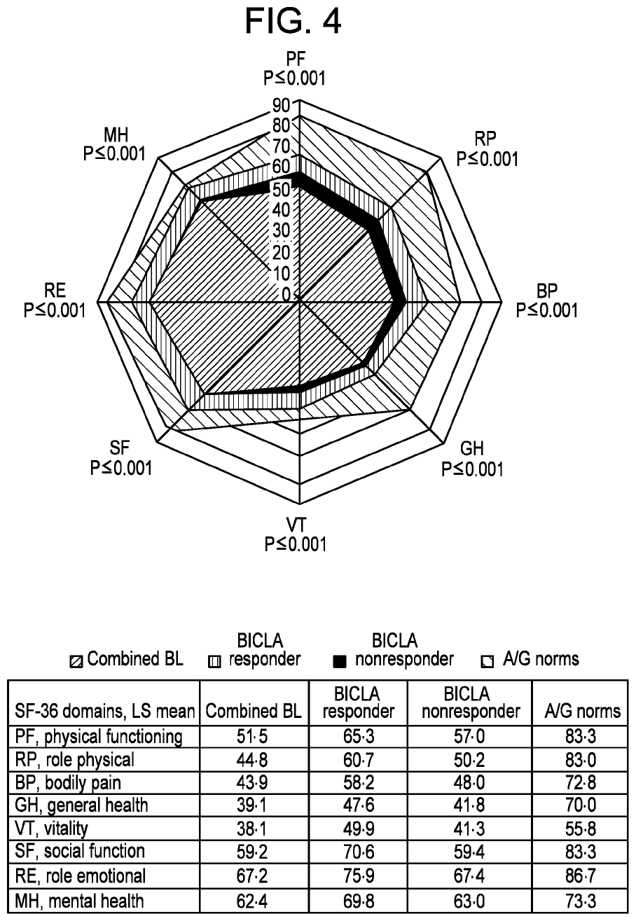

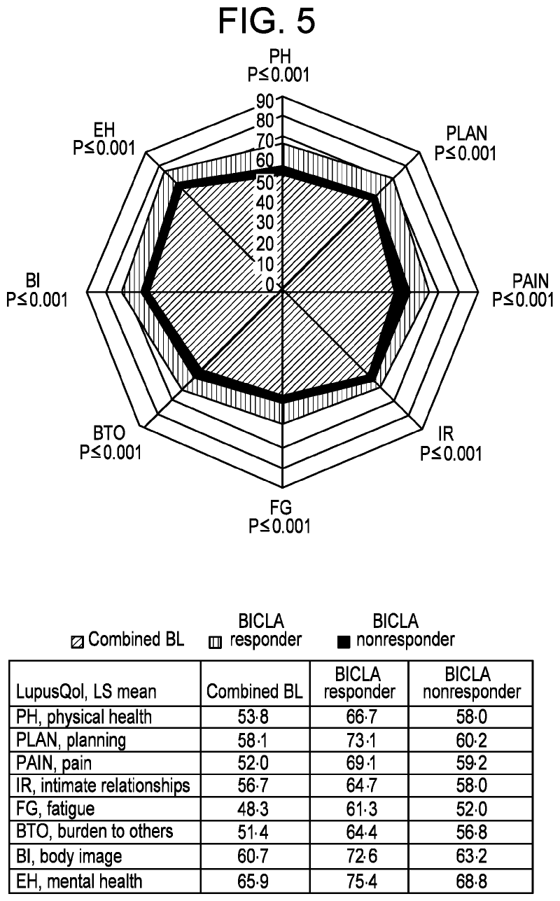

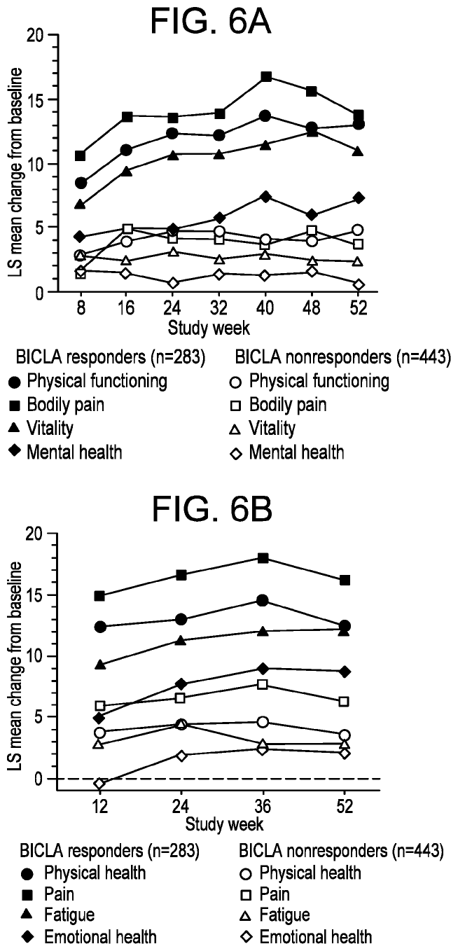

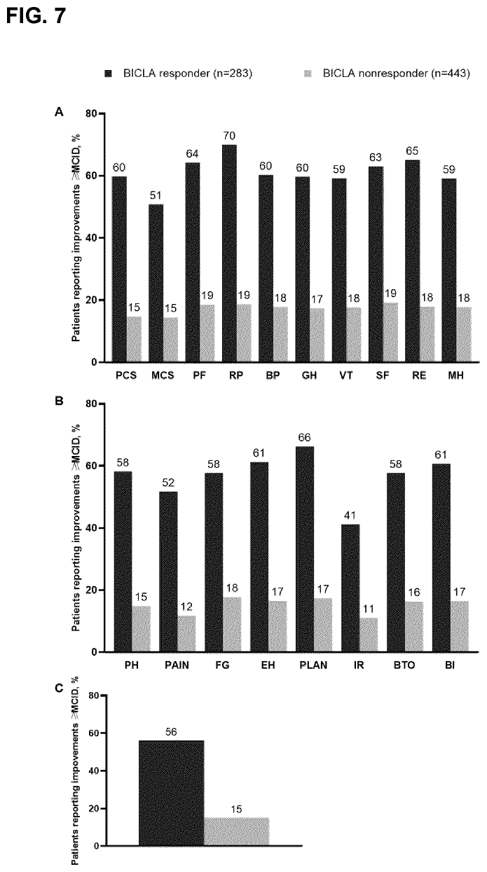

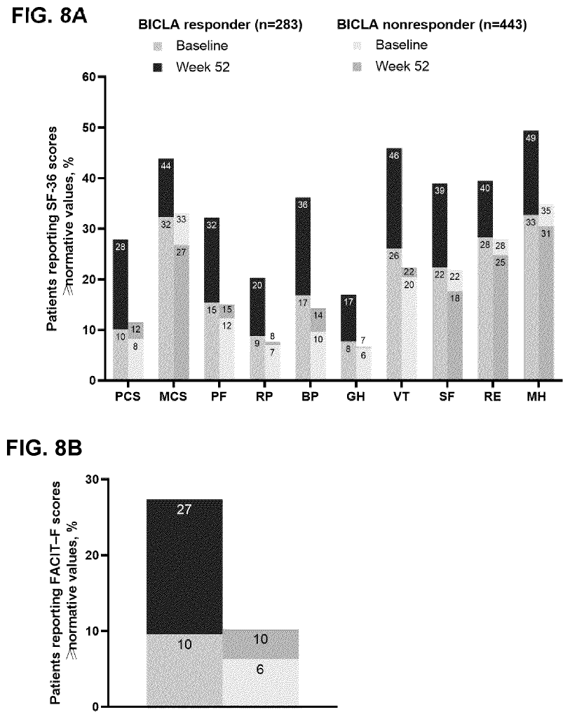

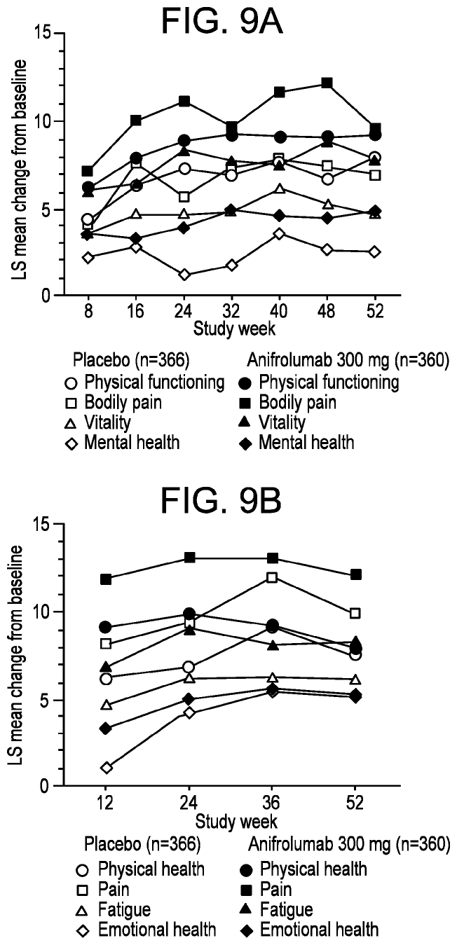

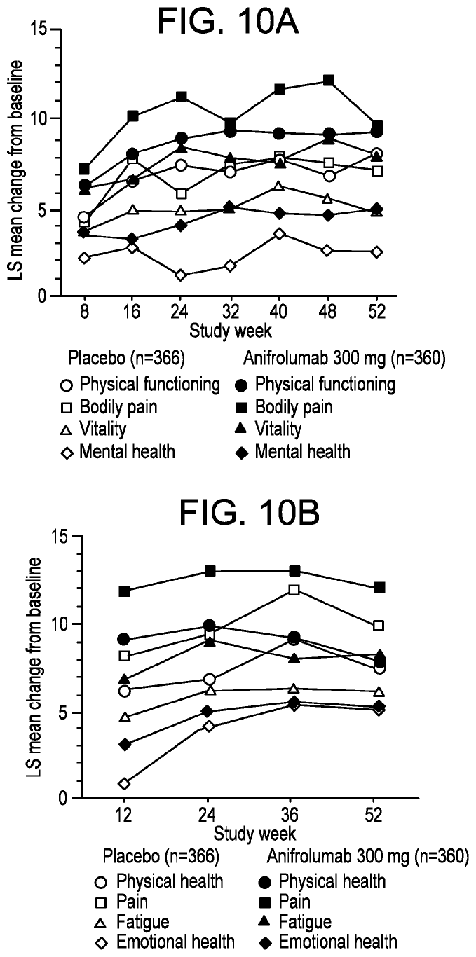

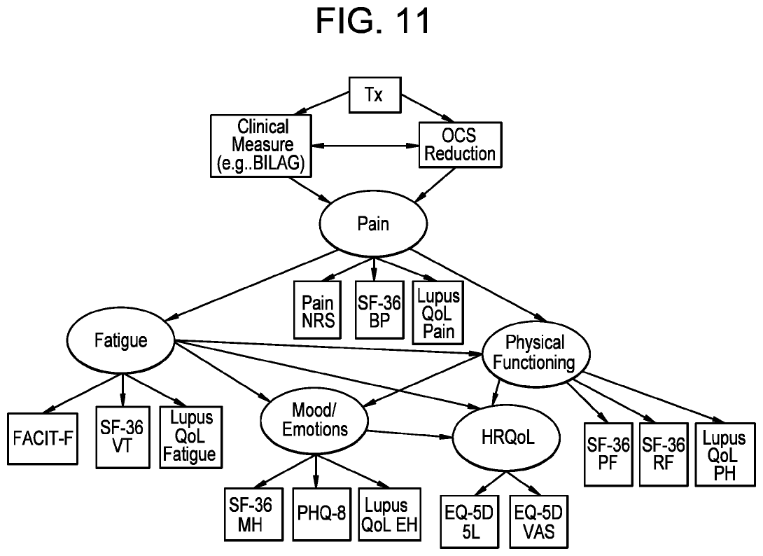

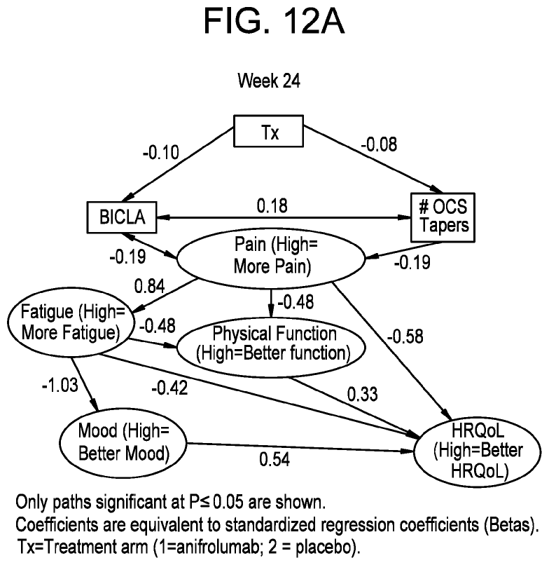

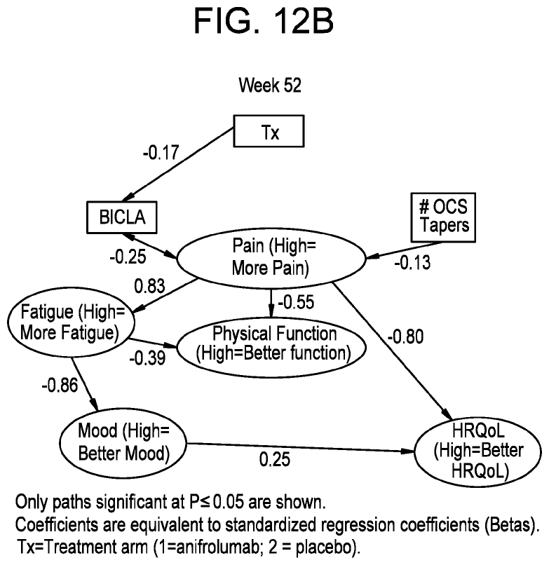

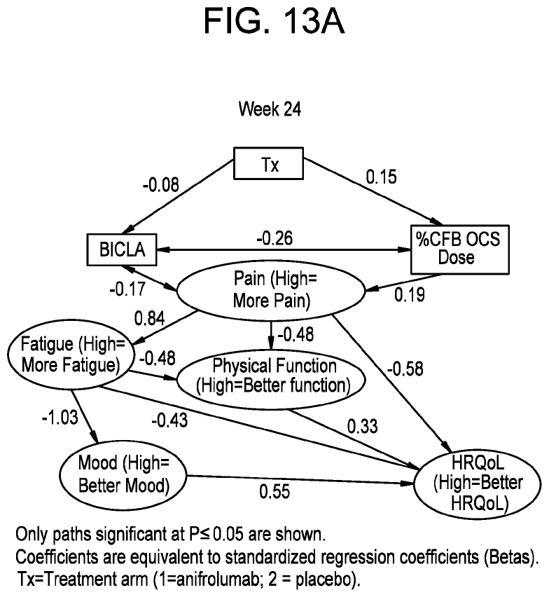

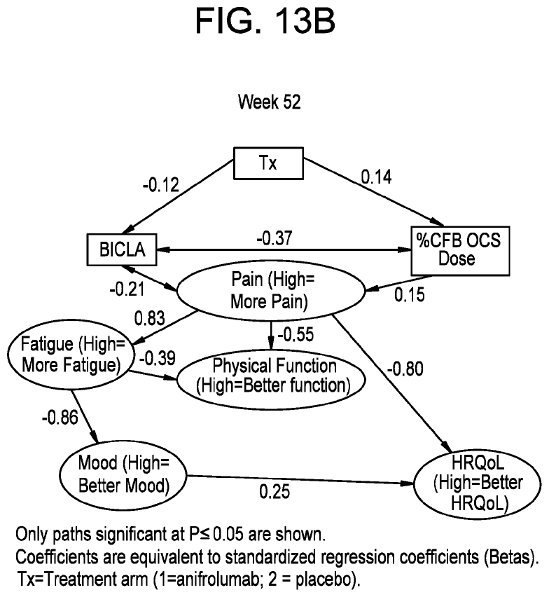

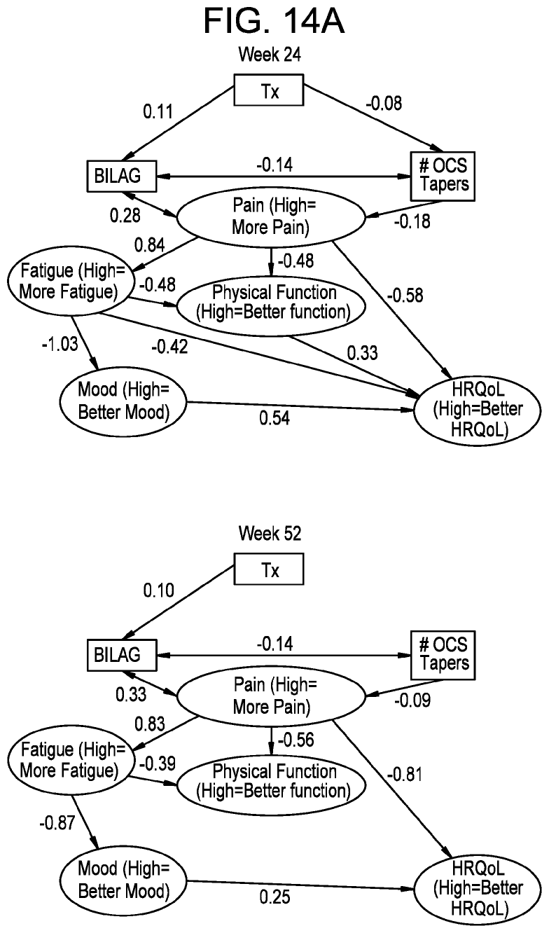

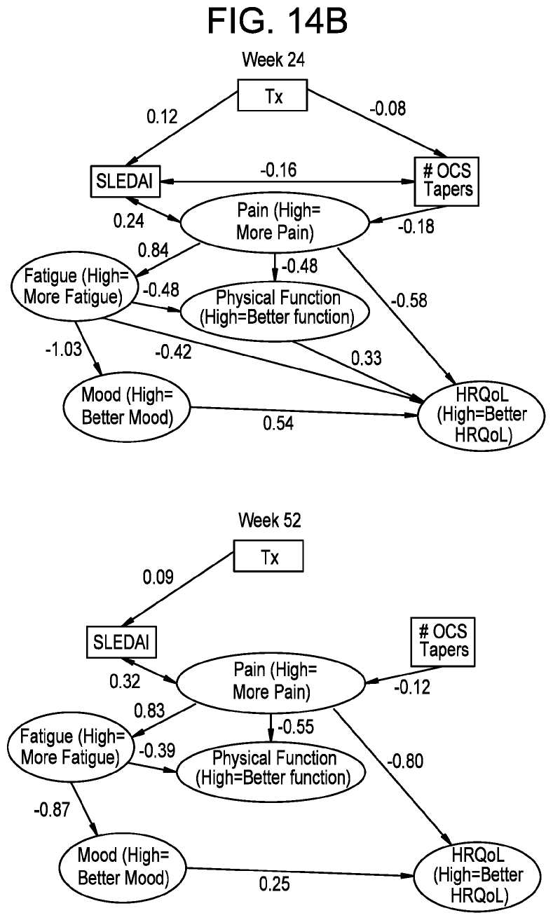

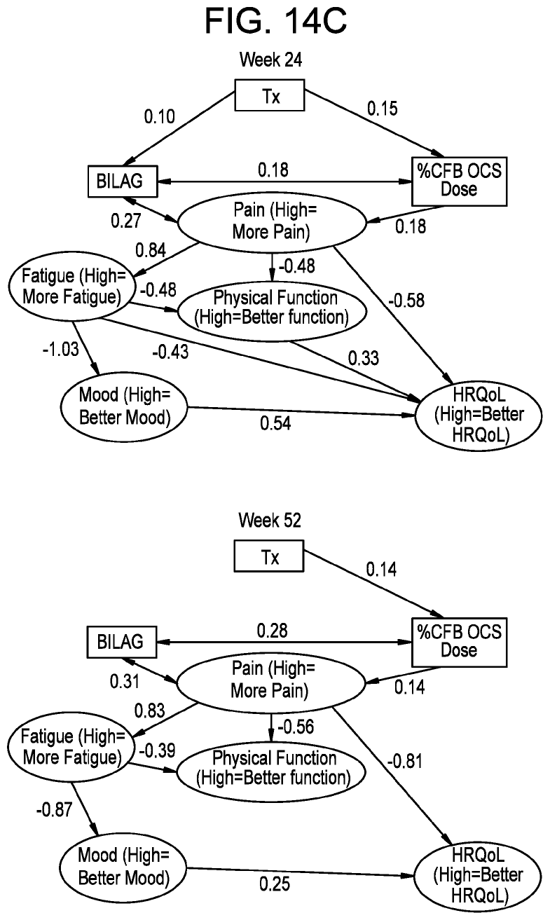

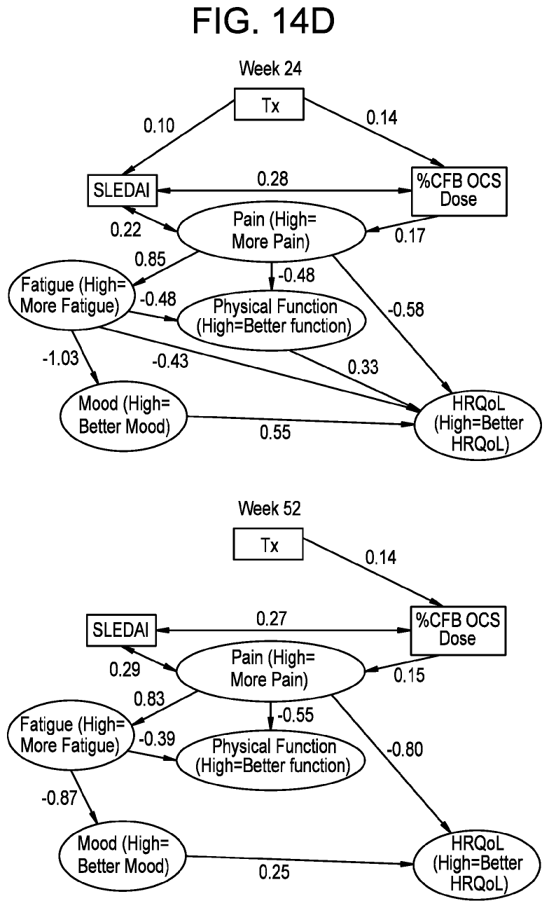

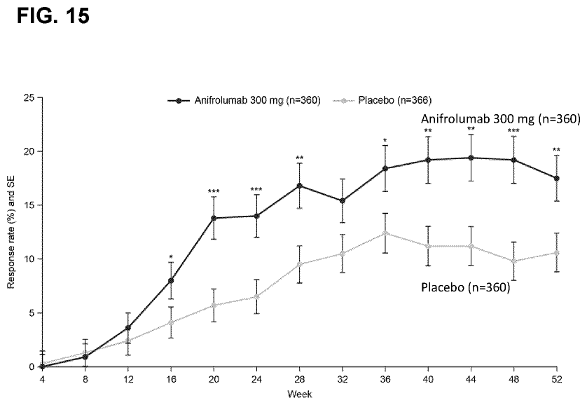

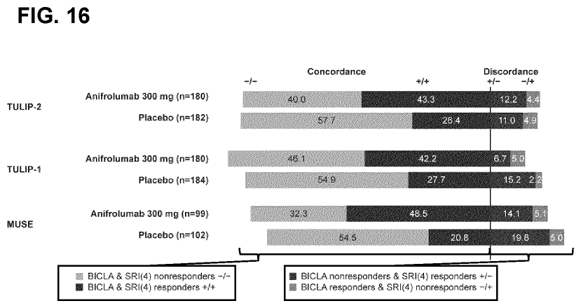

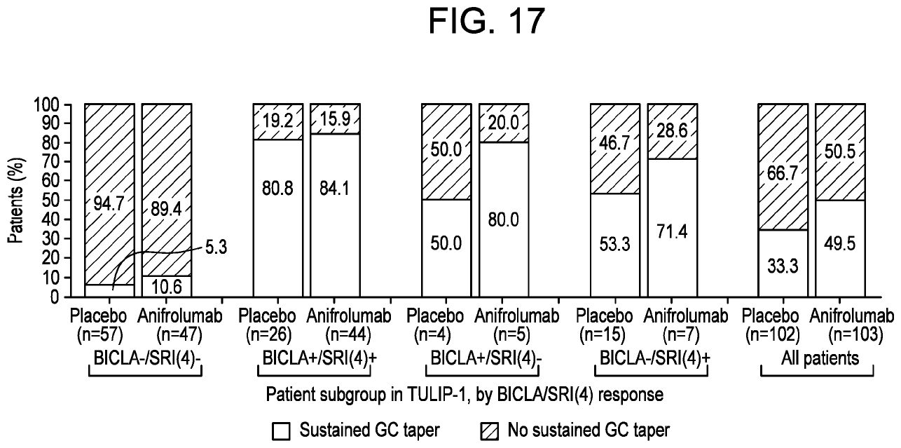

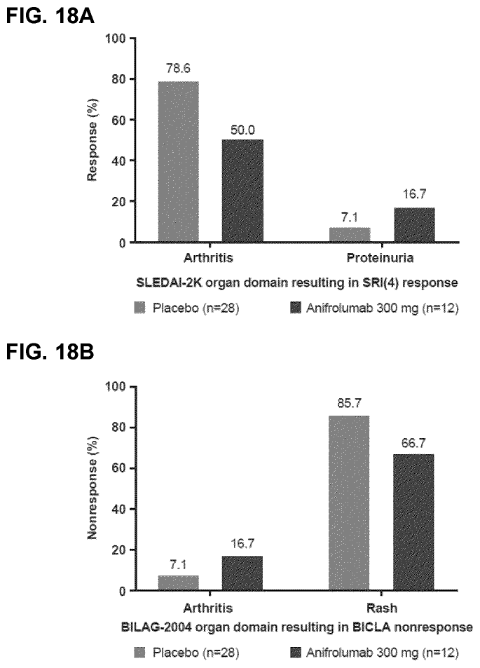

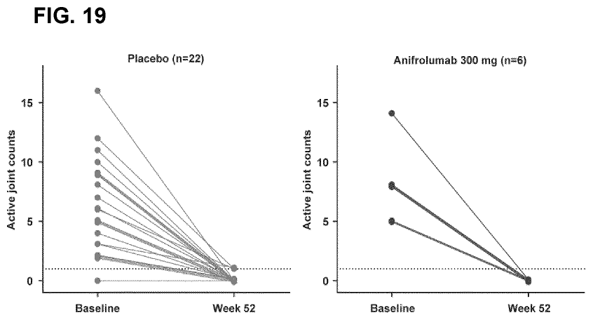

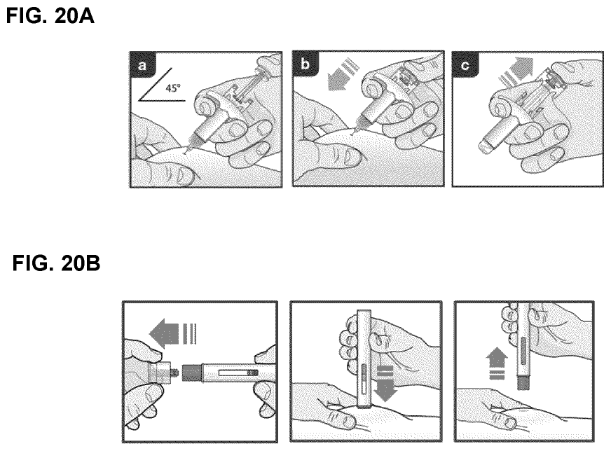

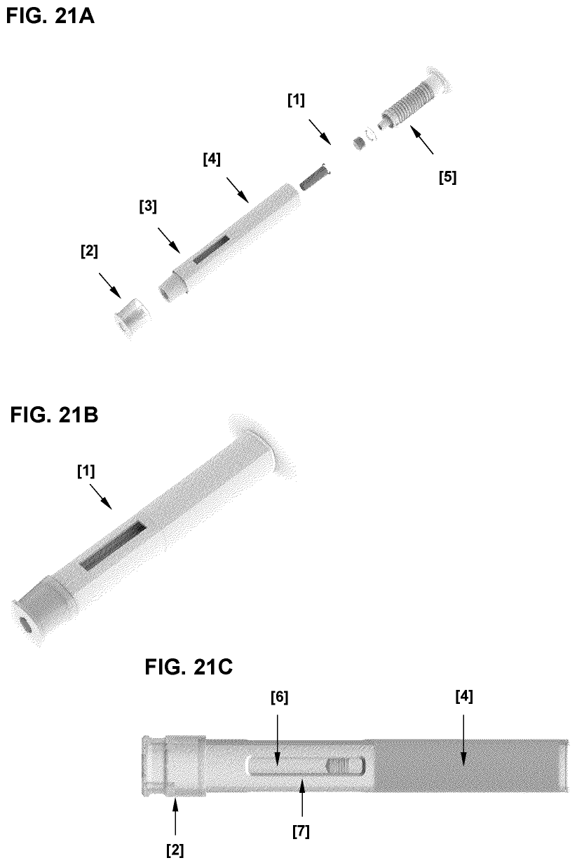

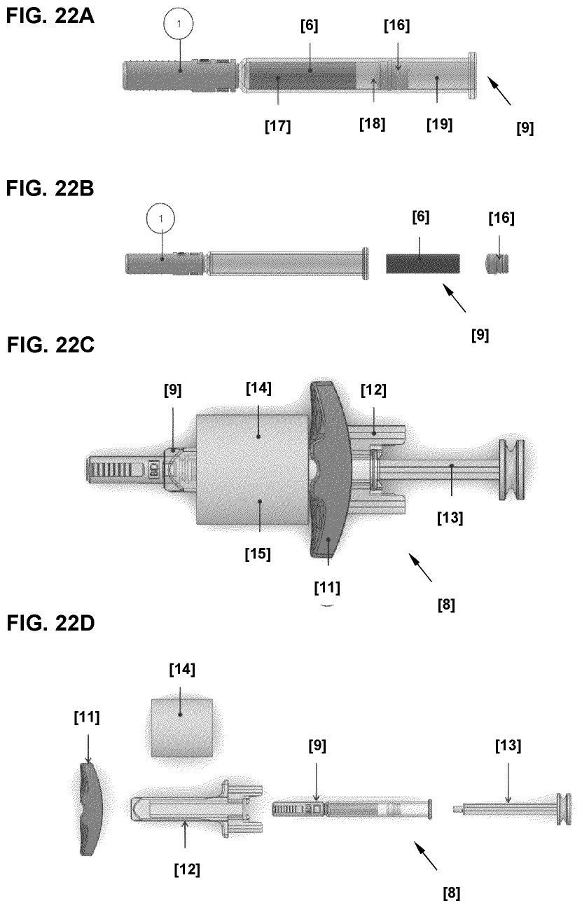

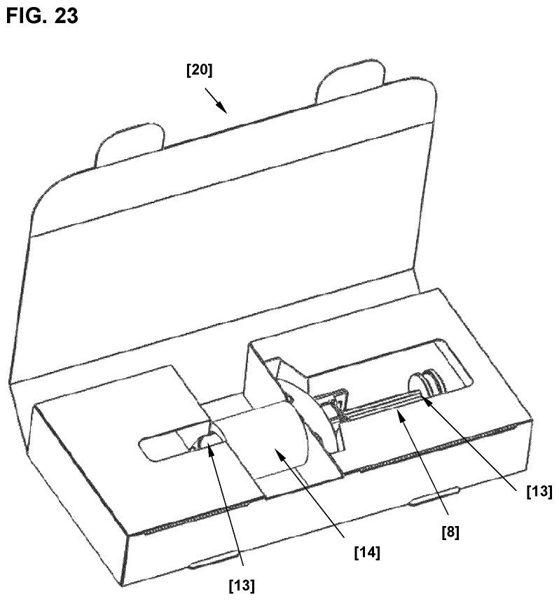

Response rate differences favouring anifrolumab 300 mg over placebo were observed for all 5 stringent BICLA endpoints across MUSE, TULIP-1, and TULIP-2 (FIG. 3). More patients met response criteria for both BICLA and SRI(4) at W52 with anifrolumab vs placebo (treatment difference, 14.3%-28.6%; nominal P≤0.004). A greater proportion of patients had BICLA responses at W52 with sustained GC taper with anifrolumab vs placebo. More patients had BICLA responses at W52 with no flares after W12 with anifrolumab vs placebo. More patients had BICLA responses at W52 with both sustained GC taper and no flares after W12 with anifrolumab vs placebo (treatment difference, 15.3%-19.3%; nominal P≤0.006). More patients attained crBICLA responses (requiring complete resolution of baseline disease activity) at W52 with anifrolumab vs placebo (treatment difference, 11.1%-14.1%; nominal P≤0.017).

Odds ratios favouring anifrolumab 300 mg over placebo were observed for all 5 endpoints at Week 52:
BICLA response+sustained GC taper, range: 1.72-3.97
BICLA response+no flares after Week 12, range: 2.30-3.47
BICLA response+no flares after Week 12+sustained GC taper, range: 2.65-4.16
Complete-resolution BICLA (crBICLA) response (requiring complete resolution of BILAG-2004 A/B scores), range: 2.45-2.74
BICLA+SRI(4) response, range: 1.89-3.76

Positive treatment differences favouring anifrolumab over placebo for crBICLA response were observed from approximately Week 32 (Week 28 in TULIP-1) and sustained through Week 52 in TULIP-2, TULIP-1, and MUSE (FIG. 3)

TABLE 8-2
Patient Demographics and Baseline Clinical Characteristics

| | TULIP-2 | | TULIP-1 | | MUSE | |
|---|---|---|---|---|---|---|
| | | Anifrolumab | | Anifrolumab | | Anifrolumab |
| | Placebo | 300 mg | Placebo | 300 mg | Placebo | 300 mg |
| | (n = 182) | (n = 180) | (n = 184) | (n = 180) | (n = 102) | (n = 99) |
| Age, mean (SD), years | 41.1 (11.5) | 43.1 (12.0) | 41.0 (12.3) | 42.0 (12.0) | 39.3 (12.9) | 39.1 (11.9) |
| Female, n (%) | 170 (93.4) | 168 (93.3) | 171 (92.9) | 165 (91.7) | 93 (91.2) | 93 (93.9) |
| Race, n (%) | | | | | | |
| White | 107 (58.8) | 110 (61.1) | 137 (74.5) | 125 (69.4) | 41 (40.2) | 35 (35.4) |
| Black/AfricanAmerican | 25 (13.7) | 17 (9.4) | 23 (12.5) | 29 (16.1) | 12 (11.8) | 19 (19.2) |
| Asian | 30 (16.5) | 30 (16.7) | 5 (2.7) | 11 (6.1) | 13 (12.7) | 3 (3.0) |
| Other | 12 (6.6) | 15 (8.3) | 19 (10.3) | 15 (8.3) | 36 (35.3) | 38 (38.4) |
| BILAG-2004, n(%) | | | | | | |
| ≥1 A | 95 (52.2) | 81 (45.0) | 84 (45.7) | 93 (51.7) | 49 (48.0) | 52 (52.5) |
| 0 As, ≥2 Bs | 78 (42.9) | 91 (50.6) | 84 (45.7) | 79 (43.9) | 48 (47.1) | 41 (41.4) |
| SLEDAI-2K global | 11.5 (3.9) | 11.4 (3.6) | 11.5 (3.5) | 11.3 (4.0) | 11.1 (4.4) | 10.7 (3.7) |
| score, mean (SD) | | | | | | |
| PGA score, mean (SD) | 1.8 (0.4) | 1.7 (0.4) | 1.8 (0.4) | 1.9 (0.4) | 1.8 (0.4) | 1.9 (0.4) |
| GC,^{a} n (%) | 151 (83.0) | 141 (78.3) | 153 (83.2) | 150 (83.3) | 88 (86.3) | 79 (79.8) |
| ≥10 mg/day | 83 (45.6) | 87 (48.3) | 102 (55.4) | 103 (57.2) | 64 (62.7) | 55 (55.6) |

BILAG, British Isles Lupus Assessment Group; GC, glucocorticoid; PGA, Physician's Global Assessment; SD, standard deviation; SLEDAI-2K, Systemic Lupus Erythematosus Disease Activity Index 2000.
^{a}Prednisone or equivalent.

### 8.5 Conclusions

In phase 2 and 3 trials in patients with SLE, anifrolumab treatment was consistently associated with improved disease control vs placebo using 5 novel, stringent BICLA based endpoint definitions, including BICLA response with sustained GC taper and no flares, BICLA response requiring complete resolution of baseline disease activity, and dual BICLA and SRI(4) responses. crBICLA response, requiring complete resolution of all baseline BILAG-2004 A/B scores, was sustained from as early as Week 28 through Week 52. These results support the ability of anifrolumab to reduce global disease activity, control flares, and minimize GC use, key treatment goals in patients with SLE.

### 9 EXAMPLE 4: THE CLINICAL MEANINGFULNESS OF A BICLA RESPONSE IN TERMS OF PATIENT-REPORTED OUTCOMES: TREATMENT OF MODERATE TO SEVERE SLE WITH ANIFROLUMAB

### 9.1 Summary

### 9.1.1 Background

The British Isles Lupus Assessment Group-based Composite Lupus Assessment (BICLA) is a validated global measure of treatment response in systemic lupus erythematosus (SLE) clinical trials but does not include patient-reported outcomes (PROs). To understand the clinical meaningfulness of a BICLA response from the patient perspective, the inventors analysed PROs by BICLA responses and by treatment group using pooled data from the phase 3 TULIP-1 and TULIP-2 trials of anifrolumab in patients with moderate to severe SLE.

### 9.1.2 Methods

This post hoc analysis assessed health-related quality of life (HRQoL) using Short Form 36 Health Survey (SF-36-v2) and Lupus Quality of Life (LupusQoL), fatigue using Functional Assessment of Chronic Illness Therapy-Fatigue (FACIT-F), and pain numerical rating scale as well as Patient Global Assessment (PtGA) of disease activity. Changes from baseline and proportions of patients reporting PRO improvements≥minimum clinically important differences (MCID) and scores≥ normative values were compared in BICLA responders and nonresponders and by treatment group.

### 9.1.3 Findings

Of 726 patients in the TULIP-1 and TULIP-2 trials, 283 were BICLA responders and 443 were BICLA nonresponders. BICLA responders reported greater mean improvements from baseline at Week 52 in PtGA, SF-36, LupusQOL, FACIT-F, and pain (all nominal p<0.005). Compared with nonresponders, more BICLA responders reported improvements≥ MCID [range 41-70% (77/187 to 184/264) vs 11-19% (33/286 to 76/398)] and scores≥ normative values [range 17-49% (47/283 to 139/283) vs 7-31% (30/443 to 135/443)] at Week 52. As a supplementary analysis, anifrolumab-treated patients reported greater improvements in SF-36, LupusQOL, and FACIT-F compared with placebo.

### 9.1.4 Interpretation

More patients with SLE treated with anifrolumab achieved a BICLA response. BICLA responders reported statistically significant and clinically meaningful improvements in PtGA, HRQoL, fatigue, and pain compared with nonresponders. More patients with SLE receiving anifrolumab reported improved HRQoL and fatigue than placebo.

### 9.2 Research in Context

9.2.1 Evidence Before this Study

Primary endpoints in systemic lupus erythematosus (SLE) randomised controlled trials (RCTs) are generally clinician-evaluated composite measures, such as the British Isles Lupus Assessment Group-based Composite Lupus Assessment (BICLA) used in the TULIP-2 and TULIP-1 (secondary endpoint) phase 3 RCTs of anifrolumab in patients with moderate to severe SLE. To date, BICLA has been utilised in >20 SLE clinical trials. Composite responder indices, such as BICLA and SLE Responder Index-4 (SRI[4]), determine disease global improvement, ensure no meaningful worsening in any organ systems, and include a physician's global assessment, but do not provide insights into the impact of the disease or its treatment from a patient's point of view. Patient-reported outcomes (PROs) measure unique aspects of disease, and it cannot be assumed that changes in a patient's well-being are directly reflected by improvements in physician-assessed disease activity. Discordance between disease activity measures and PROs have been shown repeatedly in trials and in clinical practice. The Outcome Measures in Rheumatology (OMERACT) consensus group and US Food and Drug Administration have recommended measures of disease activity, organ damage, health-related quality of life (HRQoL), adverse events, and economic costs be utilised in SLE RCTs. Previous studies directly comparing the effect of a BICLA response with PROs have included only a general PRO measurement or only an SLE-specific PRO measurement in their analyses of BICLA responders.

9.2.2 Added Value of this Study

This post hoc analysis of pooled data from the phase 3 TULIP trials of anifrolumab assessed the clinical meaningfulness of a BICLA response from a patient's perspective based on a multidimensional approach to capture patient-reported impact of disease and its treatment: HRQoL, fatigue, pain, and patient global assessment (PtGA) of disease activity. Across all PRO measures, BICLA responders reported greater improvements compared with nonresponders which were statistically significant and clinically meaningful. Anifrolumab, in addition to standard SLE therapy, improved patient-reported physical and mental health as well as fatigue.

### 9.2.3 Implications of all Available Evidence

A BICLA response is a meaningful outcome from a patient's perspective. BICLA responders have improved disease activity, fewer flares and lower cumulative glucocorticoid dosages, and report better HRQoL, improved fatigue, and pain and disease activity compared with nonresponders. More patients treated with anifrolumab, compared to placebo, were BICLA responders, a response which was associated with clinically meaningful improvements in HRQoL, fatigue, pain, and PtGA. These findings support the use of anifrolumab as an effective treatment option for patients with moderate to severe SLE.

### 9.3 Introduction

Systemic lupus erythematosus (SLE) is a relapsing, remitting chronic autoimmune disease that impacts health-related quality of life (HRQoL) 40 and is associated with considerable morbidity and increased mortality⁴¹. Given the multisystemic and heterogeneous nature of SLE, the Outcome Measures in Rheumatology (OMERACT) international consensus effort recommended assessment of five core set domains in randomised controlled clinical trials (RCTs) and longitudinal observational studies in SLE: disease activity, organ damage, HRQoL, adverse events, and economic costs⁴².

Patient-reported outcomes (PROs) are an important aspect of evaluating therapies in RCTs as well as clinical practice and are important assessments of treatment benefits in patients with SLE⁴². Clinical and laboratory measures of SLE disease activity and damage do not assess disease and treatment-related impacts of SLE on patients' daily lives. PROs capture information such as symptom severity, disease impact, and impairments in HRQoL, and are an important complement to composite measures of response when evaluating the efficacy of SLE treatments both in practice and in RCTs.

Improvement in global disease activity in the phase 3 TULIP-1 (NCT02446912) and TULIP-2 (NCT02446899) trials of anifrolumab, a human monoclonal antibody to type I interferon (IFN) receptor subunit 1, were assessed using the British Isles Lupus Assessment Group-based Composite Lupus Assessment (BICLA)^{19,21}. BICLA responders receiving anifrolumab differed statistically from placebo at Week 52 in TULIP-1 (secondary endpoint) and TULIP-2 (primary endpoint) RCTs.

This post hoc analysis examined improvements in the above PROs reported by BICLA responders versus nonresponders regardless of treatment group assignment using pooled data from the phase 3 TULIP RCTs. PROs were also assessed by treatment group.

### 9.4 Methods

### 9.4.1 Patients and Study Design

This was a post hoc analysis of pooled data from the 52-week placebo randomised controlled trials (RCTs) TULIP-1 and TULIP-2. The trial designs and methods have been described in detail previouslyl^{9,21}. Briefly, patients with moderate to severe SLE despite standard therapy were randomised to receive anifrolumab 300 mg or placebo intravenously every 4 weeks for 48 weeks. All patients were aged 18 to 70 years and fulfilled the American College of Rheumatology classification criteria for SLE. Patients with active severe lupus nephritis or neuropsychiatric SLE were excluded.

### 9.4.2 Study Endpoints and Assessments

Patient data pooled from the TULIP-1 and TULIP-2 trials were analysed in two subgroups: 1) BICLA responders versus nonresponders (BICLA response at Week 52 regardless of treatment group assignment) and 2) treatment group (anifrolumab 300 mg vs placebo). Patients in the 150-mg treatment group in the TULIP-1 trial were not included in this analysis. BICLA responses were defined as all of the following: reduction of all baseline BILAG-2004 A and B domain scores to B/C/D and C/D, respectively, with no worsening in other BILAG-2004 organ systems; no increases in Systemic Lupus Erythematosus Disease Activity Index 2000 (SLEDAI-2K) score (from baseline to Week 52) or Physician's Global Assessment (PGA) score (≥0.3 points from baseline); no study treatment discontinuation; and no use of restricted medications beyond protocol-allowed thresholds.

We evaluated PtGA, HRQoL using SF-36 version 2 (acute) and LupusQoL, fatigue using FACIT-F, and pain. PtGA was measured on a visual analogue scale (VAS) of 0-100 mm. SF-36 measures eight domains (physical functioning, role physical, bodily pain, general health, vitality, social functioning, role emotional, and mental health). Domain scores are standardised and range from 0 (worst) to 100 (best); higher scores indicate better HRQoL. Physical and mental Component Summary (PCS and MCS) scores are calculated by weighting z-transformed and norm-based scoring of the domains, with a mean of 50 and a standard deviation of 10. The LupusQoL contains eight domains: physical health, pain, fatigue, emotional health, planning, intimate relationships, burden to others, and body image, and is designed to be used with SF-36. Scores for each of the eight domains range from 0 (worst) to 100 (best). The FACIT-F is a 13-item patient-reported measure of fatigue. All items are summed to create a single fatigue score with a range from 0 to 52; higher scores represent better functioning and/or less fatigue. 14, 15 Patient-reported pain was captured with an 11-point NRS from 0 (no pain) to 10 (worst pain imaginable).

### 9.4.3 Analysis by BICLA Response (BICLA Responders Vs Nonresponders)

Changes from baseline to Week 52 in PtGA, SF-36, LupusQOL, FACIT-F, and pain NRS were compared. Spydergrams were prepared to illustrate Week 52 scores in each SF-36 and LupusQoL domain, compared with baseline domain values and age- and gender-matched healthy US subjects (SF-36 only). Proportions of patients reporting improvements≥minimum clinically important differences (MCID) from baseline to Week 52 were evaluated for SF-36, LupusQOL, and FACIT-F. MCIDs for SF-36 PCS and MCS scores are improvements≥2.5 points and ≥5.0 points in the eight SF-36 domains, respectively. 18 MCID in LupusQOL were defined as changes from baseline≥3.4 in physical health, ≥8.5 in pain, ≥3.9 in fatigue, ≥3.4 in emotional health, ≥6.5 in planning, ≥9.2 in intimate relationships, ≥5.3 in burden to others, and ≥1.1 in body image. 19 MCID in FACIT-F is defined as ≥4.0-point improvement.20

Proportions of BICLA responders and nonresponders reporting scores≥ normative values in SF-36 and FACIT-F at Week 52 were evaluated. Normative scores age/gender matched to this protocol population for SF-36 domains include physical functioning (83.25), role physical (82.96), bodily pain (72.75), general health (69.95), vitality (55.82), social functioning (83.30), role emotional (86.68), and mental health (73.34); the normative score for FACIT-F is 43.6.21

Correlations between PRO measurements of fatigue (SF-36 vitality domain, LupusQoL fatigue domain, and FACIT-F) and pain (SF-36 bodily pain, LupusQoL pain, and pain NRS) were evaluated at Week 52 in BICLA responders and nonresponders.

### 9.4.4 Analysis by Treatment Group (Anifrolumab 300 mg Vs Placebo)

Changes from baseline to Week 52 and proportions of patients reporting improvements≥ MCID (PRO responders) at Week 52 in SF-36, LupusQOL, and FACIT-F were compared by treatment group.

### 9.4.5 Statistical Analyses

The similar designs of the TULIP-1 and TULIP-2 RCTs allowed for the results to be pooled. Comparisons of changes from baseline to Week 52 by BICLA responses and by treatment group, 95% confidence intervals (CIs), and nominal P-values used a mixed model for repeated measures with fixed effects for baseline values, group, visit, study, and stratification factors of SLEDAI-2K score at screening, Day 1 oral glucocorticoid dosage, and type I IFN gene signature test status at screening. The proportions of patients reporting improvements≥MCID in PROs and 95% CIs were calculated using a stratified Cochran-Mantel-Haenszel approach with stratification factors. Missing data were imputed using the last observation carried forward for the first visit with missing data; subsequent visits with missing data were not imputed. For responder analyses, if any component of the variable could not be derived owing to missing data, the patient was classified as a nonresponder for that visit. For assessment of pooled TULIP-1 and TULIP-2 data, TULIP-1 data were analysed according to the TULIP-2 restricted medication rules. Pearson correlation coefficients along with nominal p-values testing the hypothesis of the coefficient being equal to zero are presented. These analyses were not controlled for multiple comparisons and are presented with nominal p-values.

### 9.5 Results

### 9.5.1 Baseline Characteristics

Data were pooled from 726 patients in the TULIP-1 and TULIP-2 RCTs. Of these 726 patients, 366 received placebo (184 and 182 patients in TULIP-1 and TULIP-2, respectively) and 360 received anifrolumab 300 mg (180 patients in each trial). In total, there were 283 BICLA responders and 443 BICLA nonresponders, regardless of treatment group assignment. Patient demographics and baseline disease characteristics were generally balanced across BICLA responders and nonresponders (Table 9-1) and anifrolumab 300 mg and placebo groups (Table 9-2), except that a greater proportion of BICLA responders had ≥1 BILAG-2004 A item and a lower proportion had SLEDAI-2K scores≥10. Baseline scores for PtGA, SF-36 (including PCS, MCS, and the eight domains), LupusQoL domains, FACIT-F, and pain were generally similar across BICLA responders and nonresponders (Table 9-1) and anifrolumab and placebo groups (Table 9-2).

TABLE 9-1
Baseline patient demographics, disease characteristics, and
SLE medications in patients enrolled in TULIP-1 and TULIP-2
(pooled data) by BICLA responders vs nonresponders

| | BICLA responder | | BICLA nonresponder | |
|---|---|---|---|---|
| | (n = 283) | | (n = 443) | |
| Age, mean (SD), years | 41.6 | (11.8) | 41.9 | (12.0) |
| Female, n (%) | 261 | (92) | 413 | (97) |

Race, n (%)

| | | | | |
|---|---|---|---|---|
| White | 189 | (67) | 290 | (66) |
| Black/African American | 38 | (13) | 56 | (13) |
| Asian | 27 | (10) | 49 | (11) |
| American Indian/Alaska Native | 4 | (1) | 2 | (1) |
| Other | 17 | (6) | 38 | (9) |
| Time from initial SLE diagnosis to | 109 | (95) | 118 | (102) |
| randomization, median (range), months | | | | |

BILAG-2004, n (%)

| | | | | |
|---|---|---|---|---|
| ≥1 A item | 156 | (55) | 197 | (45) |
| No A items and ≥2 B items | 108 | (38) | 224 | (51) |

SLEDAI-2K

| | | | | |
|---|---|---|---|---|
| Mean (SD) ≥ 10, | 10.7 | (3.1) | 11.9 | (4.1) |
| n (%) | 186 | (66) | 334 | (75) |
| PGA, mean (SD) | 1.76 | (0.4) | 1.80 | (0.4) |
| PROs | | | | |
| PtGA, mean (SD) | 54.9 | (23.1) | 55.4 | (21.3) |
| SF-36 PCS, mean (SD) | 38.3 | (9.2) | 36.9 | (9.2) |
| SF-36 MCS, mean (SD) | 43.8 | (11.4) | 44.2 | (11.3) |
| SF-36 domains, mean (SD) | | | | |
| Physical functioning | 53.0 | (26.4) | 50.6 | (26.8) |
| Role physical | 45.0 | (26.9) | 44.7 | (25.3) |
| Bodily pain | 45.8 | (23.3) | 42.6 | (21.0) |
| General health | 41.2 | (18.2) | 37.8 | (18.4) |
| Vitality | 39.3 | (22.1) | 37.3 | (20.7) |
| Social function | 59.5 | (26.5) | 58.9 | (26.7) |
| Role emotional | 66.7 | (27.4) | 67.5 | (27.3) |
| Mental health | 62.0 | (21.0) | 62.6 | (21.1) |
| LupusQoL domains, mean (SD) | | | | |
| Physical health | 53.8 | (26.5) | 53.8 | (25.2) |
| Pain | 53.7 | (29.0) | 50.9 | (28.7) |
| Fatigue | 50.1 | (26.5) | 47.1 | (26.5) |
| Emotional health | 66.0 | (25.1) | 65.9 | (24.7) |
| Planning | 57.8 | (31.3) | 58.3 | (30.0) |
| Intimate relationships | 55.8 | (32.8) | 57.3 | (30.7) |
| Burden to others | 52.4 | (31.5) | 50.8 | (30.2) |
| Body image | 61.2 | (28.8) | 60.4 | (29.2) |
| FACIT-F, mean (SD) | 26.1 | (12.4) | 25.6 | (11.9) |
| Pain NRS, mean (SD) | 5.2 | (2.5) | 5.7 | (2.4) |

BICLA, British Isles Lupus Assessment Group-based Composite Lupus Assessment; BILAG-2004, British Isles Lupus Assessment Group-2004; FACIT-F, Functional Assessment of Chronic Illness Therapy-Fatigue; LupusQoL, Lupus Quality of Life; MCS, mental component summary; NRS, numerical rating scale; PCS, physical component summary; PGA, Physician's Global Assessment; PROs, patient- reported outcomes; PtGA, Patient's Global Assessment; SD, standard deviation; SF-36, Short Form 36 Health Survey; SLE, systemic lupus erythematosus; SLEDAI-2K, Systemic Lupus Erythematosus Disease Activity Index 2000.

TABLE 9-2
Baseline patient demographics, disease characteristics, and SLE medications
in patients enrolled in TULIP-1 and TULIP-2 (pooled data) by treatment group

| | | | Anifrolumab | |
|---|---|---|---|---|
| | Placebo | | 300 mg | |
| | (n = 366) | | (n = 360) | |
| Age, mean (SD), years | 41.0 | (11.9) | 42.6 | (12.0) |
| Female, n (%) | 341 | (93) | 333 | (93) |
| Race, n (%) | | | | |
| White | 244 | (67) | 235 | (65) |
| Black/African American | 48 | (13) | 46 | (13) |
| Asian | 35 | (10) | 41 | (11) |
| American Indian/Alaska Native | 2 | (1) | 4 | (1) |
| Other | 29 | (8) | 26 | (7) |
| Time from initial SLE diagnosis to randomization, | 79 | (4-503) | 91 | (0-555) |
| median (range), months | | | | |

BILAG-2004, n (%)

| | | | | |
|---|---|---|---|---|
| ≥1 A item | 179 | (49) | 174 | (48) |
| No A items and ≥2 B items | 162 | (44) | 170 | (47) |

SLEDAI-2K

| | | | | |
|---|---|---|---|---|
| Mean (SD) ≥ 10, | 11.5 | (3.7) | 11.4 | (3.8) |
| n (%) | 266 | (73) | 254 | (71) |
| PGA, mean (SD) | 1.8 | (0.4) | 1.8 | (0.4) |
| PROS | | | | |
| SF-36 PCS, mean (SD) | 37.4 | (9.2) | 37.6 | (9.3) |
| SF-36 MCS, mean (SD) | 44.0 | (11.6) | 44.0 | (11.1) |
| SF-36 domains, mean (SD) | | | | |
| Physical functioning | 50.9 | (26.9) | 52.2 | (26.4) |
| Role physical | 44.9 | (24.6) | 44.7 | (27.2) |
| Bodily pain | 43.8 | (22.7) | 43.9 | (21.4) |
| General health | 39.8 | (18.2) | 38.5 | (18.6) |
| Vitality | 39.0 | (20.5) | 37.2 | (21.9) |
| Social function | 59.6 | (25.9) | 58.7 | (27.4) |
| Role emotional | 66.6 | (26.5) | 67.8 | (28.1) |
| Mental health | 62.1 | (21.0) | 62.6 | (21.1) |
| LupusQoL domains, mean (SD) | | | | |
| Physical health | 53.4 | (25.7) | 54.1 | (25.7) |
| Pain | 51.5 | (29.0) | 52.5 | (28.7) |
| Fatigue | 47.7 | (26.4) | 48.9 | (26.7) |
| Emotional health | 64.6 | (25.5) | 67.3 | (24.1) |
| Planning | 57.8 | (30.4) | 58.4 | (30.6) |
| Intimate relationships | 57.8 | (30.1) | 55.6 | (33.0) |
| Burden to others | 51.0 | (31.1) | 51.9 | (30.4) |
| Body image | 62.0 | (28.2) | 59.4 | (29.8) |
| FACIT-F, mean (SD) | 25.9 | (12.0) | 25.7 | (12.2) |

BILAG-2004, British Isles Lupus Assessment Group-2004; FACIT-F, Functional Assessment of Chronic Illness Therapy-Fatigue; LupusQoL, Lupus Quality of Life; MCS, mental component summary; PCS, physical component summary; PGA, Physician's Global Assessment; PROs, patient-reported outcomes; SD, standard deviation; SF-36, Short Form 36 Health Survey; SLE, systemic lupus erythematosus; SLEDAI-2K, Systemic Lupus Erythematosus Disease Activity Index 2000.

### 9.5.2 PROs in BICLA Responders vs Nonresponders

Greater improvements in PtGA scores from baseline to Week 52 were reported by BICLA responders than nonresponders (least squares [LS] mean −16.1 vs −4.2, nominal p<0.0001) (Table 9-3). FIG. 4 and FIG. 5 spydergrams summarise LS mean SF-36 and LupusQoL domain scores reported by BICLA responders versus nonresponders at baseline and Week 52. At Week 52, BICLA responders reported greater improvements in PCS, MCS, and all eight SF-36 domains than nonresponders (all nominal p<0.001) (FIG. 4 and Table 9-3), and in all LupusQoL domains (all nominal p<0.001; intimate relationships, nominal p=0.005) (FIG. 5 and Table 9-3). LS mean improvements in FACIT-F and pain NRS scores reported by BICLA responders were greater than nonresponders at Week 52 (both nominal p<0.001) (Table 9-3). In comparable domains of SF-36 (physical functioning, bodily pain, vitality, and mental health) and LupusQOL (physical health, pain, fatigue, and emotional health), improvements from baseline reported by BICLA responders occurred at the first evaluation post treatment and were maintained to Week 52 (FIG. 6).

TABLE 9-3
LS mean change from baseline at Week 52 in PROs PtGA, SF-
36, LupusQOL, FACIT-F, and pain NRS by BICLA responders
vs nonresponders in TULIP-1 and TULIP-2 (pooled data)

| | BICLA | | BICLA | | | | |
|---|---|---|---|---|---|---|---|
| | responder, | | nonresponder, | | | | |
| | change from | | change from | | | | |
| | baseline, LS | | baseline, LS | | LS mean | | |
| | mean (SE) | | mean (SE) | | difference | | Nominal |
| | (n = 283) | | (n = 443) | | (95% CI) | | p-value |
| PtGA | −16.1 | (1.6) | −4.2 | (1.5) | −11.8 | (−15.9 to −7.8) | <0.0001 |
| SF-36 | | | | | | | |
| SF-36 PCS | 5.2 | (0.5) | 2.0 | (0.5) | 3.2 | (2.0 to 4.4) | <0.001 |
| SF-36 MCS | 3.4 | (0.6) | −0.3 | (0.6) | 3.7 | (2.2 to 5.3) | <0.001 |
| SF-36 domains | | | | | | | |
| Physical functioning | 13.2 | (1.4) | 4.8 | (1.3) | 8.3 | (5.0 to 11.6) | <0.001 |
| Role physical | 15.4 | (1.4) | 4.8 | (1.4) | 10.6 | (7.1 to 14.1) | <0.001 |
| Bodily pain | 13.8 | (1.4) | 3.6 | (1.4) | 10.1 | (6.6 to 13.7) | <0.001 |
| General health | 7.8 | (1.0) | 2.0 | (1.0) | 5.9 | (3.3 to 8.4) | <0.001 |
| Vitality | 11.0 | (1.3) | 2.3 | (1.2) | 8.7 | (5.5 to 11.8) | <0.001 |
| Social functioning | 11.2 | (1.5) | 0.03 | (1.4) | 11.1 | (7.4 to 14.9) | <0.001 |
| Role emotional | 8.7 | (1.4) | 0.2 | (1.3) | 8.5 | (5.0 to 12.0) | <0.001 |
| Mental health | 7.3 | (1.1) | 0.5 | (1.1) | 6.8 | (3.9 to 9.6) | <0.001 |
| LupusQoL | | | | | | | |
| Physical health | 12.4 | (1.2) | 3.6 | (1.2) | 8.8 | (5.8 to 11.8) | <0.001 |
| Pain | 16.2 | (1.4) | 6.3 | (1.4) | 9.9 | (6.4 to 13.4) | <0.001 |
| Fatigue | 12.1 | (1.4) | 2.9 | (1.4) | 9.3 | (5.8 to 12.8) | <0.001 |
| Emotional health | 8.7 | (1.3) | 2.1 | (1.3) | 6.7 | (3.4 to 9.9) | <0.001 |
| Planning | 14.8 | (1.5) | 1.9 | (1.4) | 12.9 | (9.3 to 16.5) | <0.001 |
| Intimate relationships | 8.9 | (1.9) | 2.2 | (1.9) | 6.7 | (2.0 to 11.4) | 0.005 |
| Burden to others | 12.8 | (1.6) | 5.2 | (1.6) | 7.7 | (3.6 to 11.7) | <0.001 |
| Body image | 12.8 | (1.5) | 3.4 | (1.5) | 9.5 | (5.7 to 13.2) | <0.001 |
| FACIT-F | 7.2 | (0.6) | 1.7 | (0.6) | 5.5 | (3.9 to 7.0) | <0.001 |
| Pain NRS | −1.5 | (0.1) | −0.3 | (0.1) | −1.1 | (−1.5 to −0.8) | <0.0001 |

BICLA, British Isles Lupus Assessment Group-based Composite Lupus Assessment; CI, confidence interval; FACIT-F, Functional Assessment of Chronic Illness Therapy-Fatigue; LS, least squares; LupusQoL, Lupus Quality of Life; MCS, mental component summary; NRS, numerical rating scale; PCS, physical component summary; PROs, patient-reported outcomes; PtGA, patient's global assessment; SE, standard error; SF-36, Short Form 36 Health Survey.

At Week 52, a greater proportion of BICLA responders compared with nonresponders reported clinically meaningful improvements≥MCID in PCS, MCS, and all eight domains of SF-36, ranging from 51 to 70% (140/276 [MCS] to 184/264 [role physical]) versus 15 to 19% (63/416 [PCS] to 76/398 [role physical]) (FIG. 7A), and all LupusQoL domains: 41 to 66% (77/187 [intimate relationships] to 152/231 [planning]) vs 11 to 18% (33/286 [intimate relationships] to 74/410 [fatigue]) (FIG. 7B). A greater proportion of BICLA responders compared with nonresponders reported FACIT-F improvements≥MCID at Week 52: 56% (155/276) versus 15% (66/439) (FIG. 7C).

At baseline, the proportions of patients reporting SF-36 and FACIT-F scores≥normative values were low (<35%) in both BICLA responders and nonresponders. However, the proportions of BICLA responders reporting scores≥ normative values at baseline were generally higher compared with BICLA nonresponders, especially in SF-36 MCS and bodily pain, vitality, and social functioning domain scores (FIG. 8). At Week 52, a greater proportion of BICLA responders compared with nonresponders reported scores≥normative values in PCS, MCS, and all eight domains of SF-36:17 to 49% (47/283 [general health] to 139/283 [mental health]) versus 7 to 31% (30/443 [general health] to 135/443 [mental health]) and FACIT-F (FIG. 8): 27% (76/283) versus 10% (46/443). The proportion of BICLA responders reporting scores≥normative values at Week 52 more than doubled from baseline in the four physical SF-36 domains (physical functioning, role physical, bodily pain, and general health), as well as the vitality domain, and consequently PCS scores, whereas the proportion of BICLA nonresponders reporting scores≥normative values at Week 52 decreased from baseline in the social functioning, role emotional, and mental health domains and consequently MCS scores.

Correlation analyses of the measurements of patient-reported fatigue showed that SF-36 vitality domain, LupusQOL fatigue domain, and FACIT-F were strongly associated, irrespective of BICLA responder group (correlation coefficient>0.75, all nominal p<0.0001), with the strongest correlation evident between FACIT-F and LupusQoL fatigue (Table 9-4). Similar results were observed in patient-reported pain. SF-36 bodily pain and LupusQoL pain domains were strongly correlated in both BICLA responders and nonresponders (correlation coefficient 0.72 and 0.67, both nominal p<0.0001) (Table 9-4). Pain NRS was strongly negatively correlated with SF-36 bodily pain and LupusQoL pain domains (all nominal p<0.0001).

TABLE 9-4
Pearson correlations between PRO measurements for fatigue (SF-36 vitality, LupusQoL
fatigue, and FACIT-F) and pain (SF-36 bodily pain, LupusQoL pain, and pain NRS)
by BICLA responders versus nonresponders in TULIP-1 and TULIP-2 (pooled data)

| | |
|---|---|
| BICLA responder | BICLA nonresponder |
| (n = 283) | (n = 443) |

Fatigue, Pearson correlation coefficient (95% CI); nominal p-value

| | | |
|---|---|---|
| FACIT-F vs SF-36 vitality | 0.85 (0.81 to 0.88); <0.0001 | 0.84 (0.80 to 0.87); <0.0001 |
| FACIT-F vs LupusQoL fatigue | 0.86 (0.82 to 0.89); <0.0001 | 0.85 (0.82 to 0.88); <0.0001 |
| SF-36-v2 (acute) vitality vs | 0.75 (0.69 to 0.80); <0.0001 | 0.76 (0.71 to 0.81); <0.0001 |
| LupusQoL fatigue | | |

Pain, Pearson correlation coefficient (95% CI); nominal p-value

| | | |
|---|---|---|
| Pain NRS vs SF-36 bodily pain | −0.82 (−0.86 to −0.78); <0.0001 | −0.80 (−0.84 to −0.75); <0.0001 |
| Pain NRS vs LupusQoL pain | −0.76 (−0.81 to −0.70); <0.0001 | −0.70 (−0.75 to −0.63); <0.0001 |
| SF-36 bodily pain vs LupusQoL pain | 0.72 (0.65 to 0.77); <0.0001 | 0.67 (0.60 to 0.73); <0.0001 |

BICLA, British Isles Lupus Assessment Group-based Composite Lupus Assessment; CI, confidence interval; FACIT-F, Functional Assessment of Chronic Illness Therapy-Fatigue; LupusQoL, Lupus Quality of Life; NRS, numerical rating scale; PROs, patient-reported outcomes; SF-36, Short Form 36 Health Survey.

### 9.5.3 PROs Reported by Patients Receiving Anifrolumab 300 mg Versus Placebo

LS mean changes from baseline in SF-36, LupusQOL, and FACIT-F observed in patients who received anifrolumab were numerically greater compared with placebo across all domains (Table 9-5). Over time, anifrolumab-treated patients, compared with placebo, reported numerically greater LS mean changes from baseline at first evaluation in four SF-36 domains (physical functioning, bodily pain, vitality, and mental health; Week 8) and LupusQoL comparable domains (physical health, pain, fatigue, and emotional health; Week 12). These differences were maintained to Week 52, except in the LupusQoL physical health and emotional health domains (FIG. 9).

TABLE 9-5
LS mean changes from baseline at Week 52 in SF-36, LupusQOL, and
FACIT-F by treatment group in TULIP-1 and TULIP-2 (pooled data)

| | Placebo, | | Anifrolumab 300 mg, | | | |
|---|---|---|---|---|---|---|
| | change from | | change from | | | |
| | baseline, LS | | baseline, LS | | LS mean | |
| | mean (SE) | | mean (SE) | | difference | |
| | (n = 366) | | (n = 360) | | (95% CI) | |
| SF-36 | | | | | | |
| SF-36 PCS | 3.2 | (0.5) | 3.8 | (0.5) | 0.6 | (−0.5 to 1.8) |
| SF-36 MCS | 0.7 | (0.6) | 2.1 | (0.6) | 1.3 | (−0.2 to 2.9) |
| SF-36 domains | | | | | | |
| Physical functioning | 7.9 | (1.3) | 9.2 | (1.3) | 1.3 | (−2.1 to 4.6) |
| Role physical | 7.9 | (1.4) | 11.4 | (1.4) | 3.4 | (−0.1 to 7.0) |
| Bodily pain | 7.0 | (1.4) | 9.6 | (1.4) | 2.6 | (−1.0 to 6.2) |
| General health | 4.2 | (1.0) | 5.1 | (1.0) | 0.8 | (−1.8 to 3.4) |
| Vitality | 4.7 | (1.3) | 7.9 | (1.3) | 3.2 | (−0.03 to 6.4) |
| Social functioning | 3.6 | (1.5) | 6.8 | (1.5) | 3.2 | (−0.6 to 7.0) |
| Role emotional | 3.2 | (1.4) | 5.2 | (1.4) | 2.0 | (−1.5 to 5.5) |
| Mental health | 2.5 | (1.1) | 4.9 | (1.1) | 2.4 | (−0.4 to 5.3) |
| LupusQoL | | | | | | |
| Physical health | 7.4 | (1.3) | 7.9 | (1.2) | 0.4 | (−2.6 to 3.5) |
| Pain | 9.9 | (1.4) | 12.1 | (1.4) | 2.2 | (−1.4 to 5.7) |
| Fatigue | 6.1 | (1.4) | 8.3 | (1.4) | 2.1 | (−1.4 to 5.7) |
| Emotional health | 5.1 | (1.3) | 5.3 | (1.3) | 0.2 | (−3.1 to 3.5) |
| Planning | 6.8 | (1.5) | 9.2 | (1.5) | 2.4 | (−1.4 to 6.1) |
| Intimate relationships | 4.5 | (1.9) | 6.5 | (1.9) | 2.0 | (−2.7 to 6.7) |
| Burden to others | 8.1 | (1.6) | 9.4 | (1.6) | 1.4 | (−2.7 to 5.4) |
| Body image | 7.0 | (1.5) | 8.5 | (1.5) | 1.5 | (−2.3 to 5.4) |
| FACIT-F | 3.4 | (0.6) | 4.9 | (0.6) | 1.5 | (−0.1 to 3.2) |

At Week 52, a numerically greater proportion of anifrolumab-treated patients compared with placebo reported clinically meaningful improvements≥MCID in PCS, MCS, and all eight domains of SF-36, all LupusQoL domains, and FACIT-F (FIG. 10). Treatment differences were greatest in SF-36 domains than LupusQoL domains, and smallest in the LupusQoL intimate relationships domain.

### 9.6 Discussion

Patients with SLE report substantial impairments in HRQoL and persistent fatigue. In the phase 3 TULIP-1 and TULIP-2 RCTs, baseline HRQoL and fatigue scores were low and PtGA and pain scores were high, reflecting the significant impact of this disease. Response to treatment in the TULIP RCTs was measured using the composite responder index BICLA, developed for use as a clinical trial endpoint to measure improvement or worsening in disease activity, which does not include PROs. Thus, the impact and meaning for patients meeting desired treatment targets are not captured. Compared with placebo, more patients treated with anifrolumab were BICLA responders in the TULIP RCTs. Thus, the aim of these analyses was to evaluate the clinical meaningfulness of being a BICLA responder from a patient perspective. In addition to reductions in disease activity, the analyses reported here show that, compared with nonresponders, BICLA responders in the TULIP RCTs reported greater improvements in PtGA; SF-36 PCS, MCS, and all eight domains; all LupusQoL domains; FACIT-F; and pain scores that were clinically meaningful. Also, the proportions of patients classified as PRO responders determined by reported improvements≥ MCID in SF-36, LupusQOL, and FACIT-F were greater in BICLA responders than nonresponders. Finally at Week 52, more BICLA responders compared with nonresponders reported scores≥normative values in SF-36 PCS, MCS, and FACIT-F, and matched normative scores in all SF-36 domains.

Discordance can often exist between clinical measures of disease activity in SLE and patients' perceptions of their disease and its impact on HRQoL. Our analyses support the clinical meaningfulness of being a BICLA responder in patients with moderate to severe SLE from a patient perspective and reflect the global benefit of improved disease activity.

OMERACT recommends that generic (SF-36) and disease-specific (LupusQoL) HRQoL and fatigue instruments be used together as complementary evaluations in RCTs and longitudinal observational studies in patients with SLE. Based on published analyses, all three instruments (SF-36, LupusQOL, and FACIT-F) are reliable, valid, responsive, and sensitive to changes in patients with SLE enrolled in RCTs and are recommended for use as supportive measures in trials and LOS. SF-36, LupusQOL, and FACIT-F are responsive to perceived changes in health status according to patients with SLE, and comparable domains have been shown to be strongly correlated. As illustrated by our analyses, patients who were BICLA responders reported greater improvements in these PROs that were clinically meaningful to patients and there were large differences favouring BICLA responders in the proportions of patients reporting improvements in HRQoL and fatigue at levels equal to or above the well-being of the general population.

In contrast to the generic SF-36, LupusQoL includes specific aspects of health status that are important to patients with SLE, such as body image and appearance, self-confidence, and social support, that are incorporated into the four domains: planning, burden to others, body image, and intimate relationships. More BICLA responders than nonresponders reported greater improvements and responses in lupus-specific domains as well as domains of SF-36 which correlate with LupusQoL. These improvements were reported by BICLA responders as early as 4-12 weeks after treatment initiation and generally increased over time.

Fatigue and pain are the most common clinical manifestations of SLE, and are associated with poor physical and mental functioning with a significant impact on daily life. Mean improvements in FACIT-F scores were greater in BICLA responders than nonresponders. Similarly, BICLA responders also reported greater improvements in the SF-36 vitality domain, which correlates with FACIT-F14 and the LupusQOL fatigue domain. The findings of our correlation analyses indicate strong correlations between these three measurements of patient-reported fatigue. Additionally, consistent improvements reported by BICLA responders versus nonresponders in patient-reported pain measures were highly correlated. With anifrolumab treatment, more patients with moderate to severe SLE achieved a BICLA response in both TULIP RCTs. In these analyses, HRQoL and fatigue benefits reported by BICLA responders were also observed with anifrolumab treatment compared with placebo. Although treatment differences were small in several domains, more patients treated with anifrolumab reported clinically meaningful improvements in all three PRO instruments (SF-36, LupusQOL, and FACIT-F).

In conclusion, in patients with moderate to severe SLE, BICLA responders reported improvements in disease activity, HRQoL, fatigue, and pain. These results support the importance of the BICLA responder definition in terms of patients' experiences and impacts of their disease as well as other clinical responses, such as fewer flares, sustained oral glucocorticoid tapering, and less medical resource utilisation. These data also suggest that reductions in disease activity with anifrolumab treatment improve patient-reported HRQoL, fatigue, and pain.

### 10 EXAMPLE 5: A “CAUSAL CASCADE” AMONG OUTCOMES WITH ANIFROLUMAB FOR PATIENTS WITH SYSTEMIC LUPUS ERYTHEMATOSUS: EXAMINING THE DIRECT AND INDIRECT RELATIONSHIPS AMONG TREATMENT, CLINICAL FACTORS, SYMPTOMS, PATIENT FUNCTIONING, AND HEALTH-RELATED QUALITY OF LIFE

### 10.1 Summary

### 10.1.1 Background

Systemic lupus erythematosus (SLE) significantly impairs health-related quality of life (HRQoL), and patient-reported outcomes (PROs) measure a unique aspect of the disease not captured by disease activity. Measuring direct treatment benefit with PROs in patients with SLE in a clinical trial setting is difficult. In this post hoc analysis, structural equation modelling was used to examine the “causal cascade” of interaction between anifrolumab, disease activity, and PROs in pooled data from the phase 3 TULIP-1 and TULIP-2 trials.

### 10.1.2 Methods

Data were pooled from the TULIP-1 (n=364) and TULIP-2 (n=362) trials, which were randomized, placebo-controlled, 52-week trials of intravenous anifrolumab (300 mg every 4 weeks for 48 weeks). We evaluated changes from baseline to Week 24 and Week 52 in four clinical (BICLA, BILAG-2004, SLEDAI-2K, and changes in glucocorticoid dosage) and six PRO measures (SF-36, FACIT-F, EQ-5D, LupusQOL, PHQ-8, and pain NRS) in our hypothesized model of interactions.

### 10.1.3 Results

Our hypothesized model had an acceptable fit to the pooled TULIP trial data. At Week 24, significant paths revealed that when compared with placebo, anifrolumab treatment had direct effect on improvement in disease activity as measured by BICLA, BILAG-2004, SLEDAI-2K, and changes to glucocorticoid dosage. In turn, these clinical measures reduced pain, which improved fatigue, physical functioning, mood/emotions, and HRQoL. When the model incorporated number of glucocorticoid tapers as the measure of change in glucocorticoid dosage, treatment effects of anifrolumab on glucocorticoid tapers were not retained at Week 52. However, at Week 52 treatment indirectly improved HRQoL through its direct effects on BICLA.

### 10.1.4 Conclusions

Anifrolumab is associated with significant patient-reported improvements in aspects of HRQoL including pain, fatigue, mood, and physical function. These benefits are from the direct effect of anifrolumab treatment on disease activity and reduction in glucocorticoid dosage.

### 10.2 Introduction

Systemic lupus erythematosus (SLE) is a complex, chronic, and heterogeneous autoimmune disease that can affect any organ system, and patients with SLE present with a variety of clinical manifestations⁴³. These clinical manifestations are devastating for a majority of patients and can lead to reduced physical function, loss of employment, a major impact on health-related quality of life (HRQoL), frequent hospitalizations, cumulative and irreversible organ damage, and early mortality. Organ damage accumulates from SLE disease activity itself and the treatment-related adverse effects associated with the chronic use of glucocorticoids and other immunosuppressive agents. There remains a substantial unmet medical need for novel treatments in SLE with disease-specific mechanisms of action that reduce overall disease activity and the concomitant use of steroids and other non-specific immunosuppressive agents, while also reducing flares, comorbidities, and long-term organ damage.

Most SLE clinical studies that evaluate new therapeutic options include a composite endpoint composed of clinical and laboratory measures of global and organ-specific disease and clinician-graded severity assessments as a primary endpoint. Although improvement in these clinician-rated outcomes provides evidence to support the therapeutic value of an investigational drug, they do not directly capture how patients feel and function. As such, PROs are an important complement to composite measures of disease activity and damage when evaluating the efficacy of SLE treatments.

The present inventors hypothesized a “causal cascade” of effects such that treatment with anifrolumab would result in improvements in clinical assessments of disease activity, reduced use of glucocorticoids, and improved HRQoL. In this hypothesized “causal cascade” it is important to consider the possible mechanism of action and the intervening variables in the causal chain between treatment and patient function and treatment. Thus, while there may not be a significant direct relationship between treatment and HRQoL, there may be a significant indirect relationship between treatment and HRQoL through these intervening variables.

The goal of the present analysis was to examine a causal cascade (i.e., the direct and indirect relationships) among clinical assessments, symptoms, patient functioning, and HRQoL for patients with SLE treated with anifrolumab versus placebo in the TULIP trials.

### 10.3 Methods

This was a post hoc analysis of pooled data from the phase 3 randomized, placebo-controlled, double-blind, 52-week TULIP-1 and TULIP-2 trials. Patients were aged 18 to 70 years and fulfilled the American College of Rheumatology revised classification criteria for SLE⁴⁴. Patients were randomized to receive intravenous infusions of placebo or anifrolumab (300 mg) every 4 weeks for 48 weeks in addition to standard therapy. For patients receiving oral glucocorticoids≥10 mg/day (prednisone or equivalent) at baseline, a protocol-mandated attempt to taper to ≤7.5 mg/day was required between Weeks 8 and 40; tapering was also permitted for patients receiving oral glucocorticoids<10 mg/day at baseline. Stable oral glucocorticoid dosage was required in all patients between Weeks 40 and 52.

### 10.3.1 Outcomes

The inventors evaluated four clinical and six PRO measures. The clinical measures included the British Isles Lupus Assessment Group-based Composite Lupus Assessment (BICLA)^{44,45}, British Isles Lupus Assessment Group-2004 (BILAG-2004)⁴⁶, Systemic Lupus Erythematosus Disease Activity Index 2000 (SLEDAI-2K)^{48,49}, and changes to glucocorticoid dosage measured in two different ways: (1) number of glucocorticoid dosage tapers and (2) percentage glucocorticoid dosage change from baseline. The PRO measures included the Short Form 36 Health Survey (SF-36) version 2 (acute), the Functional Assessment of Chronic Illness Therapy-Fatigue (FACIT-F), the EuroQol 5 Dimension 5 Level (EQ-5D-5L) and visual analog scale (EQ-5D VAS)^{47,48}, the Lupus Quality of Life (LupusQoL)³⁴, the eight-item Patient Health Questionnaire depression scale (PHQ-8)³⁶, and a pain Numerical Rating Scale (NRS).

### 10.3.2 Clinical and PRO Measures

### 10.3.2.1 BILAG-Based Composite Lupus Assessment (BICLA)

For a patient to be classified as a BICLA responder, improvements (partial or complete) were required in all BILAG-2004 organ systems affected by the disease at baseline. Specifically, BICLA response was achieved if the following criteria were met: reduction of all baseline BILAG-2004 A to B/C/D and baseline BILAG-2004 B to C/D, and no BILAG worsening in other organ systems (defined as 1 new BILAG-2004 A or more than 1 new BILAG-2004 B item; no worsening from baseline in SLEDAI-2K, defined as an increase from baseline of >0 points; no worsening from baseline in the patients' lupus disease activity defined by an increase ≥0.3 points on Physician's Global Assessment Visual Analog Scale (scale 0-3); no discontinuation of investigational product; and no use of restricted medications beyond the protocol-allowed threshold before assessment.

### 10.3.2.2 British Isles Lupus Assessment Group-2004 (BILAG-2004)

The BILAG-2004 is a translational index with 9 organ systems (general, mucocutaneous, neuropsychiatric, musculoskeletal, cardiorespiratory, gastrointestinal, ophthalmic, renal, and haematology) that is able to capture changing severity of clinical manifestations. It is based on the principle of physicians' intention to treat and categorizes disease activity across the different organ systems into 5 different levels from A (severe) to E (no current or previous disease activity). Although BILAG-2004 was developed based on the principle of intention to treat, the treatment has no bearing on the scoring index; only the presence of active manifestations influences the scoring.

### 10.3.2.3 Systemic Lupus Erythematosus Disease Activity Index 2000 (SLEDAI-2K)

The SLEDAI-2K consists of a list of organ manifestations, each with a definition. A certified investigator or designated physician will complete the SLEDAI-2K assessment and decide whether each manifestation is “present” or “absent” in the last 4 weeks. The assessment also includes the collection of blood and urine for assessment of the laboratory categories in the SLEDAI-2K.

The SLEDAI-2K assessment consists of 24 lupus-related items. It is a weighted instrument, in which descriptors are multiplied by a particular organ's “weight.” For example, renal descriptors are multiplied by 4 and central nervous descriptors by 8, and these weighted organ manifestations are totaled into the final score. The SLEDAI-2K score range is 0 to 105 points with 0 indicating inactive disease. The SLEDAI-2K scores are valid, reliable, and sensitive clinical assessments of lupus disease activity. The SLEDAI-2K calculated using a timeframe of 30 days prior to a visit for clinical and laboratory values has been shown to be similar to the SLEDAI-2K with a 10-day window. A timeframe of 28 days was used in this study.

### 10.3.2.4 Glucocorticoid Reduction

A key secondary endpoint in the TULIP trials was glucocorticoid tapering to a target of ≤7.5 mg/day at Week 40 and maintained through Week 52. Glucocorticoid tapering to ≤7.5 mg/day was to be attempted in all patients with a baseline glucocorticoid dosage≥10 mg/day between Weeks 8 and 40, per protocol. Attempts to taper could be made in patients receiving glucocorticoids<10 mg/day. Stable glucocorticoid dosage was required between Week 40 and Week 52. For the current analyses, changes in glucocorticoid use during the trial were measured in two different ways: (1) a count of the number of times a patient tapered between Weeks 8 and 24 and between Weeks 8 and 40, and (2) the percentage change from baseline in glucocorticoid dosage between baseline and Week 24 and between baseline and Week 52.

### 10.3.2.5 Short Form 36 Version 2 (Acute Form; SF-36-V2)

The Short Form 36, Version 2 (SF-36-v2) (acute) is a multipurpose, 36-item survey that measures 8 domains of health: physical functioning, role limitations due to physical health, bodily pain, general health perceptions, vitality, social functioning, role limitations due to emotional problems, and mental health. It yields scale scores for each of these 8 health domains, and 2 summary measures of physical and mental health: Physical Component Summary (PCS) and Mental Component Summary (MCS). The SF-36 acute form has a one-week recall period.

### 10.3.2.6 Functional Assessment of Chronic Illness Therapy-Fatigue

The Functional Assessment of Chronic Illness Therapy-Fatigue (FACIT-F) is a 13-item patient-completed questionnaire to assess the impact of fatigue over the previous 7 days. The responses range from 0 (not at all) to 4 (very much). Final scores are derived as the sum of the final item scores and range from 0 to 52; higher total scores indicate better HRQoL³². Changes in scores>3 points are considered clinically meaningful⁴⁹.

### 10.3.2.7 EuroQol 5 Dimension 5 Level (EQ-5D-5L)

The EuroQoL 5 Dimension (EQ-5D-5L) is comprised of the following 5 dimensions: mobility, self-care, usual activities, pain/discomfort, and anxiety/depression³³. For each dimension, patients are asked to select the one response option that best describes their health today. Each dimension has 5 response options (no problems, slight problems, moderate problems, severe problems, and unable to/extreme problems) that reflect increasing levels of difficulty. The questionnaire also includes a visual analog scale (VAS), where the patients were asked to rate their health on a scale of 0-100, with 0 being worst imaginable health state and 100 being best imaginable health state.

### 10.3.2.8 Lupus Quality of Life (LupusQoL)

LupusQoL is a 34-item SLE-specific HRQoL measure. It was developed in the United Kingdom for use in adults with SLE³⁴ and was further validated in the USA. The instrument consists of 8 domains (physical health [8 items], pain [3 items], planning [3 items], intimate relationships [2 items], burden to others [3 items], emotional health [6 items], body image [5 items], and fatigue [4 items]). For each item, patients are asked to select the response that is closest to how often they have felt over the last four weeks.

### 10.3.2.9 Eight-Item Patient Health Questionnaire Depression Scale (PHQ-8)

Depression was captured with the standardized and validated PHQ-8, which consists of eight of the nine criteria on which the DSM-IV diagnosis of depressive disorders is based³⁶. The scores for each item are summed to produce a total score between 0 (no depressive symptoms) and 24 points (severe depression) with a 2-week recall period.

### 10.3.2.10 Pain Numerical Rating Scale (Pain NRS)

Overall patient-reported pain was captured with an 11-point NRS (0=no pain; 10=worst pain imaginable) with a 1-week recall period.

### 10.3.3 Patient-Reported Concepts

The PROs were further divided into 5 key patient-reported concepts of HRQoL (pain, fatigue, physical functioning, mood/emotions, and HRQoL) and were measured using a multiple indicator modeling approach where multiple PROs and PRO domains measured each underlying concept (Table 10-1). Of the 8 domains captured by SF-36, physical functioning, role physical, bodily pain, vitality, and mental health were used in the key concepts. The LupusQoL also captures 8 domains, of which, physical health, pain, fatigue, and emotional health were used in the key concepts.

TABLE 10-1
Concepts and Their Respective Measures for the Planned Analyses

| Concepts | Measure(s) |
|---|---|
| Pain | Pain NRS, SF-36 Bodily Pain, |
| | LupusQoL Pain |
| Fatigue | FACIT-Fatigue, SF-36 Vitality, |
| | LupusQoL Fatigue |
| Physical functioning | SF-36 Physical Functioning, SF-36 |
| | Role Functioning, LupusQoL |
| | Physical |
| Mood/emotions | SF-36 Mental Health, PHQ-8, |
| | LupusQoL Emotional Health |
| HRQoL | EQ-5D-5L, EQ-5D VAS |

EQ-5D-5L, EuroQol 5 Dimension 5 Level; EQ-5D VAS, EuroQol 5 Dimensions visual analog scale; FACIT-F, Functional Assessment of Chronic Illness Therapy-Fatigue; HRQoL, health-related quality of life; LupusQoL, Lupus Quality of Life; NRS, Numerical Rating Scale; PHQ-8, eight-item Patient Health Questionnaire depression scale; PRO, patient-reported outcomes; SF-36, Short Form 36 Health Survey.

### 10.3.4 Analyses

Structural equation modeling (SEM) path analysis was used to evaluate the direct and indirect effects of the clinical and PRO variables. Analyses were first conducted in each trial separately and included only patients in the 300 mg and placebo arms. TULIP-1 included an anifrolumab 150 mg group that was not included in this analysis. If results were similar in each trial, then the two trials would be pooled. Analyses were conducted using data at baseline and Weeks 24 and 52, since these three time points included all PROs of interest. All SEM analyses were conducted using Mplus version 8.4 (Muthen & Muthen, Los Angeles, CA).

### 10.3.5 Hypothesized Model

FIG. 11 presents the hypothesized model that was the basis of the present analyses. After baseline, treatment was hypothesized as having direct effects on the clinical measures of BICLA, BILAG-2004, and SLEDAI-2K responses, and was anticipated to result in reductions in glucocorticoid dosage. In addition, a correlation (the double-headed arrow) between clinical measures and glucocorticoid reduction was anticipated, such that if treatment results in a BICLA response and an improved SLEDAI-2K/BILAG-2004 score, this should also be associated with a reduction in glucocorticoid dosage.

In this model, the clinical measures and glucocorticoid dosage reduction were expected to directly affect pain. That is, if treatment worked as anticipated, it would result in improved clinical scores and glucocorticoid dosage reductions, which would lead to reduced patient-reported pain. With reductions in patient-reported pain, there would be reductions in fatigue and improved physical function. With less fatigue and improved physical functioning, we expected to see improvement in mood and ultimately improved HRQoL (FIG. 11).

This model is applied to post-baseline visits at Week 24 and 52 to examine the differential effects of treatment. No baseline model or analysis is presented because no treatment effect is observed at baseline. Four tests of goodness-of-fit (model chi-square with its degrees of freedom and P-value, comparative fit index [CFI]⁵⁰ root mean square error of approximation [RMSEA]⁵¹, and standardized root mean square residual [SRMR] were used to evaluate the correspondence of the hypothesized model to the observed data.

### 10.4 RESULTS

In total, 726 patients were included in the model, 364 from TULIP-1 and 362 from TULIP-2. Of these 726 patients, 366 received placebo (184 in TULIP-1, 182 in TULIP-2), and 360 received anifrolumab 300 mg (180 patients in each trial). Pooled patient demographics and clinical characteristics were generally balanced across treatment groups (anifrolumab 300 mg and placebo) in both individual trials and pooled patient data (Table 10-2). In the anifrolumab and placebo groups at baseline, 80.8% and 83.1% of patients, respectively, were receiving glucocorticoids, and 52.8% and 50.5% were receiving glucocorticoids≥10 mg/day.

TABLE 10-2
Baseline Demographics and Clinical Characteristics
of Patients in TULIP-1 and TULIP-2 (Pooled Data)

| | Placebo | | Anifrolumab 300 mg | |
|---|---|---|---|---|
| | (n = 366) | | (n = 360) | |
| Age, mean (SD), years | 41.0 | (11.9) | 42.6 | (12.0) |
| Female, n (%) | 341 | (93.2) | 333 | (92.5) |
| Race,^{a} n (%) | | | | |
| White | 244 | (66.7) | 235 | (65.3) |
| Asian | 35 | (9.6) | 41 | (11.4) |
| Black/African American | 48 | (13.1) | 46 | (12.8) |
| Other | 31 | (8.5) | 30 | (8.3) |
| Weight, mean (SD), kg | 73.47 | (19.62) | 72.83 | (18.38) |
| BMI, mean (SD) | 27.82 | (6.87) | 27.47 | (6.761) |
| Time from SLE diagnosis to randomization, | 78.5 | (4-503) | 91.0 | (0-555) |
| median (range), months | | | | |
| ≥1 BILAG-2004 A, n (%) | 179 | (48.9) | 174 | (48.3) |
| No BILAG-2004 A and ≥2 BILAG-2004 B, n | 162 | (44.3) | 170 | (47.2) |
| (%) | | | | |
| SLEDAI-2K global score, mean (SD) | 11.5 | (3.7) | 11.4 | (3.8) |
| SLEDAI-2K ≥10, n (%) | 266 | (72.7) | 254 | (70.6) |
| PGA score, mean (SD) | 1.8 | (0.4) | 1.8 | (0.4) |
| CLASI activity score, mean (SD) | 7.8 | (7.2) | 8.4 | (7.6) |
| Swollen joint count, mean (SD) | 7.2 | (5.7) | 6.8 | (5.8) |
| Tender joint count, mean (SD) | 10.8 | (7.5) | 10.3 | (7.4) |
| SDI score, mean (SD) | 0.6 | (0.9) | 0.6 | (1.0) |
| Baseline treatment for SLE, n (%) | 304 | (83.1) | 291 | (80.8) |
| Glucocorticoids^{b} | | | | |
| Glucocorticoids^{b} ≥10 mg/day | 185 | (50.5) | 190 | (52.8) |
| Antimalarial | 267 | (73.0) | 243 | (67.5) |
| Immunosuppressant | 177 | (48.4) | 173 | (48.1) |

BILAG-2004, British Isles Lupus Assessment Group-2004; BMI, body mass index; CLASI, Cutaneous Lupus Erythematosus Disease Area and Severity Index; PGA, Physician's Global Assessment; SD, standard deviation; SDI, Systemic Lupus International Collaborating Clinics/American College of Rheumatology Damage Index; SLE, systemic lupus erythematosus; SLEDAI-2K, SLE Disease Activity Index 2000.
^{a}Race data were missing from 16 patients in TULIP-2 (8 each in the anifrolumab and placebo groups).
^{b}Glucocorticoids include prednisone or equivalent.

The hypothesized model had an acceptable fit to the data for each trial separately, was consistent across both trials and time points, and was of acceptable size according to CFI, RMSEA, and SRMR estimates (CFI ranged from 0.931 to 0.953; RMSEA ranged from 0.065 to 0.087; SRMR ranged from 0.036 to 0.049). In addition, the relationships among the constructs were consistent across trials and time points. Therefore, the TULIP trials were pooled for these analyses. For the pooled analyses, the fit statistics were of acceptable size for the models using data at Week 24 and Week 52 that included BICLA response and the number of glucocorticoid tapers, and BICLA response and percentage change in glucocorticoid dosage (Table 10-3). The RMSEA at Week 24 was slightly elevated (preferred value≤0.08) relative to the CFI and SRMR, both of which indicated good fit (Table 10-3).

TABLE 10-3
Goodness-of-Fit Statistics in TULIP-1 and TULIP-2 (Pooled Data)

| | | | BICLA Response and | |
|---|---|---|---|---|
| | BICLA Response and Count | | Percentage Reduction in | |
| | of Glucocorticoid Tapers | | Glucocorticoid Dosage | |
| Fit Statistic: | Week 24 | Week 52 | Week 24 | Week 52 |
| Chi-square (df) | 613.245 (103)*** | 453.319 (103)*** | 616.229 (103)*** | 446.752 (103)*** |
| CFI^{a} | 0.936 | 0.949 | 0.936 | 0.951 |
| RMSEA^{b} | 0.083 | 0.068 | 0.083 | 0.068 |
| 90% CI for | 0.076-0.089 | 0.062-0.075 | 0.077-0.089 | 0.061-0.074 |
| RMSEA | | | | |
| SRMR^{c} | 0.039 | 0.035 | 0.039 | 0.034 |

***P ≤ 0.001
BICLA, British Isles Lupus Assessment Group (BILAG)-based Composite Lupus Assessment; CFI, comparative fit index; df, degrees of freedom; RMSEA, root mean square error of approximation; SRMR, standardized root mean square residual.
^{a}CFI generally preferred value ≥0.9 or 0.95
^{b}RMSEA generally preferred value ≤0.08
^{c}SRMR generally preferred value ≤0.10
For more complex models, the CFI may be smaller and the RMSEA may be larger than for more parsimonious models and with larger sample sizes.

Table 10-4 presents the model-generated mean (SD) PRO scores, number of glucocorticoid tapers, and percent change from baseline in glucocorticoid dosage at baseline (PROs only) at Weeks 24 and 52. The greatest differences in PRO scores occurred between baseline and Week 24, with only a small incremental change occurring between Weeks 24 and 52 for PROs.

TABLE 10-4
Model-Generated Mean Scores for PROs at Baseline and
Weeks 24 and 52 in TULIP-1 and TULIP-2 (Pooled Data)

| N = 726 | Baseline | | Week 24 | | Week 52 | |
|---|---|---|---|---|---|---|
| Pain, mean (SD) | | | | | | |
| Pain NRS | 5.5 | (2.4) | 4.4 | (2.6) | 4.2 | (2.6) |
| SF-36 Bodily Pain | 43.9 | (22.0) | 54.3 | (24.5) | 55.1 | (24.0) |
| LupusQoL Pain | 52.0 | (28.8) | 64.6 | (27.4) | 65.8 | (26.1) |
| Fatigue, mean (SD) | | | | | | |
| FACIT-F | 25.8 | (12.1) | 30.8 | (12.9) | 31.2 | (12.8) |
| SF-36 Vitality | 38.1 | (21.2) | 46.2 | (23.1) | 46.6 | (23.3) |
| LupusQoL Fatigue | 48.3 | (26.5) | 58.0 | (27.1) | 58.2 | (27.1) |
| Physical functioning, mean (SD) | | | | | | |
| SF-36 Physical Functioning | 51.5 | (26.6) | 61.8 | (26.0) | 63.4 | (25.9) |
| SF-36 Role Physical | 44.8 | (25.9) | 55.5 | (26.5) | 57.0 | (26.0) |
| LupusQoL Physical Health | 53.8 | (25.7) | 63.3 | (25.2) | 64.0 | (24.6) |
| Mood/Emotions, mean (SD) | | | | | | |
| SF-36 Mental Health | 62.4 | (21.0) | 65.0 | (21.6) | 66.4 | (21.4) |
| PHQ-8 | 9.7 | (6.2) | 7.9 | (6.0) | 7.8 | (6.0) |
| LupusQoL Emotional Health | 65.9 | (24.8) | 72.1 | (23.6) | 73.2 | (23.6) |
| HRQoL, mean (SD) | | | | | | |
| EQ-5D-5L | 0.6 | (0.2) | 0.7 | (0.2) | 0.7 | (0.2) |
| EQ-5D VAS | 55.6 | (20.8) | 63.8 | (21.0) | 64.7 | (21.3) |
| Glucocorticoid tapers, n, mean | | | 1.1 | (1.5) | 2.0 | (2.2) |
| (SD) | | | | | | |
| Glucocorticoid dosage, mg/day, | | | −20.0 | (44.2) | −22.6 | (52.1) |
| change from baseline, % | | | | | | |

EQ-5D VAS, EuroQol 5 Dimensions visual analog scale; EQ-5D-5L, EuroQol 5 Dimension 5 Level; FACIT-F, Functional Assessment of Chronic Illness Therapy-Fatigue; HRQoL, health-related quality of life; LupusQoL, Lupus Quality of Life; NRS, Numerical Rating Scale; PHQ-8, 8-item Patient Health Questionnaire depression scale; PRO, patient-reported outcomes; SD, standard deviation; SF-36, Short Form 36 Health Survey.

10.4.1 Relationship of PROs with BICLA Response and the Number of Glucocorticoid Dosage Tapers

FIG. 12A presents the model for Week 24 pooled data that included BICLA response and the number of glucocorticoid dosage tapers between Weeks 8 and 24 as the clinical measures. Only paths that are significant (P<0.05) are shown. The model shows that those in the anifrolumab arm were more likely to be BICLA responders and to have more glucocorticoid dosage tapers during this period. Both BICLA response and a higher number of glucocorticoid dosage tapers resulted in lower patient-reported pain.

With a reduction in pain, there was a reduction in fatigue (β=0.84), an increase in physical functioning (β=−0.48), and an improvement in HRQoL (β=−0.58). When patient-reported fatigue is lessened, there was a corresponding improvement in physical functioning (β=−0.48), an improvement in patient mood (β=−1.03), and an improvement in HRQoL (β=−0.42). When patient physical functioning was improved, there was an improvement in HRQoL (β=0.33). Finally, when mood is improved, there was an improvement in HRQoL (β=0.54) (FIG. 12A).

At Week 52, some paths did not retain the significance observed at Week 24, including between treatment and number of glucocorticoid tapers or between BICLA and number of glucocorticoid tapers (FIG. 12B). In addition, there were no longer significant relationships between HRQoL and physical functioning and fatigue. Of the remaining significant paths, the model shows that patients treated with anifrolumab are more likely to be BICLA responders, have less pain, less fatigue, better physical functioning, and better mood and HRQoL compared with patients in the placebo arm, similar to Week 24.

### 10.4.2 Relationship of PROs With BICLA Response and the Percentage Change in Glucocorticoid Dosage

Significant paths observed in our model for Week 24 in pooled data that included BICLA response and the percentage change in glucocorticoid dosage show that patients in the anifrolumab arm were more likely to be BICLA responders and to have a greater decrease in glucocorticoid dosage from baseline to Week 24 (FIG. 13A). In addition, the correlation between BICLA response and the percentage change from baseline in glucocorticoid dosage indicates that those who are BICLA responders also have a greater decrease in glucocorticoid dosage during this period. Both BICLA response and a greater decrease in glucocorticoid dosage resulted in lower patient-reported pain.

With a reduction in pain, there was a reduction in fatigue (β=0.84), an increase in physical functioning (β=−0.48), and an improvement in HRQoL (β=−0.58). When patient-reported fatigue was lessened, there is a corresponding improvement in physical functioning (β=−0.48), an improvement in patient mood (β=−1.03), and an improvement in HRQoL (β=−0.43). When patient physical functioning is improved, there was an improvement in HRQoL (β=0.33). Finally, when mood was improved, there was an improvement in HRQoL (β=0.55) (FIG. 13A).

In contrast to the model including BICLA response and the number of glucocorticoid dosage tapers, at Week 52 there are significant relationships between treatment and percentage change from baseline in glucocorticoid dosage and between BICLA response and percentage change from baseline in glucocorticoid dosage (FIG. 13B). However, there are no longer significant relationships between HRQoL and physical functioning and fatigue.

Despite these differences in the significance of the model paths, at Week 52 the model shows that patients treated with anifrolumab are more likely to be BICLA responders, have greater decreases in glucocorticoid dosage, have less pain, less fatigue, better physical functioning, and better mood and HRQoL compared with patients in the placebo arm (FIG. 13B).

10.4.3 Relationship of PROs with BILAG-2004 and SLEDAI-2K

We also evaluated the clinical measures BILAG-2004 and SLEDAI-2K in our hypothesized model at Week 24 and Week 52 for both number of glucocorticoid tapers and percentage reduction in glucocorticoid dosage (FIG. 13A-D). Results from the model were nearly identical with those presented for BICLA (FIG. 12 and FIG. 13), including significant indirect relationships between clinical response and less pain, less fatigue, better physical functioning, and better mood and HRQoL in patients treated with anifrolumab compared with patients in the placebo group (FIG. 14A-D).

### 10.5 Discussion

In this analysis, the inventors aimed to understand the relationship of treatment and disease activity with HRQoL and symptoms in patients with moderate to severe SLE enrolled in the TULIP-1 and TULIP-2 trials. The evaluation of our hypothesized model showed consistently that there is a causal cascade of effects between treatment with anifrolumab, clinical measures of disease activity, and multiple domains of HRQoL. Anifrolumab treatment did not have a direct effect on patient-reported pain or patient reported fatigue, physical functioning, mood, or HRQoL. Instead, treatment with anifrolumab, through its effect on the type 1 IFN pathway, indirectly affected these variables through its effect on clinical measures of disease activity (BICLA, BILAG-2004, SLEDAI-2K, and changes in glucocorticoid dosage).

The TULIP trials included the generic and specific HRQoL measures SF-36 and LupusQOL, and additional HRQoL measures to capture the full complement of patient health status including fatigue (FACIT-F), pain (Pain NRS), and depression (PHQ-8). These measures were incorporated into the 5 key patient-reported concepts in our model to evaluate patient-reported efficacy of anifrolumab. Our findings that anifrolumab treatment improves both clinician-rated disease activity and PROs highlights the value of multiple measures of treatment efficacy as recommended by OMERACT.

Measuring direct treatment benefit with PROs in patients with SLE in a clinical trial setting is difficult and complex. Both poor correlation between PROs and disease activity indices and discordance between patient and physician assessments of disease activity are very well known in SLE. Multiple factors including background medications, glucocorticoid use, flares, and comorbidities can confound treatment response and significantly impact HRQoL. Patients in the TULIP trials were receiving standard therapy including combinations of glucocorticoids, immunosuppressants, and antimalarials, while also attempting to taper glucocorticoids. By Week 52, patients had a mean of approximately 2 attempts to taper glucocorticoids. Additionally, 33% treated with anifrolumab and 43% of patients in the placebo group had one or more flares during the trial. Thus, these factors are likely to influence a direct assessment of anifrolumab's treatment effect on PROs. These analyses attempted to bridge the connections between treatment and PROs with hypothesized models of direct and indirect treatment effects. The significant pathways observed at Week 24 and Week 52 showed that patients treated with anifrolumab were more likely to be BICLA responders and have a greater decrease in oral glucocorticoid dosage, have less pain, less fatigue, better physical functioning, and better mood and HRQoL compared with patients in the placebo group. These results suggest that treatment with anifrolumab is effective at reducing patient-reported symptoms and improving HRQoL, though these effects are modelled indirectly.

The inventors observed a loss of some significant pathways at Week 52 compared with Week 24 in our analysis of both BICLA response and number of glucocorticoid tapers. Most significant was the loss of the pathway between treatment and glucocorticoid taper. However, significant indirect effects remained between BICLA response and pain, mood, physical function, and HRQoL. These differences in significant pathways are likely driven by the smaller differences in mean changes in the PROs between Weeks 24 and 52 than between baseline and Week 24, where most of the effects occurred. Examining reductions in oral glucocorticoids in terms of percentage change from baseline yielded somewhat more robust results at Week 52. The significant pathway between treatment and percentage change in glucocorticoid dosage presented at Week 24 was preserved at Week 52 and supports examining glucocorticoid dosage changes in multiple ways.

This analysis had limitations. The variable for the percentage change from baseline in glucocorticoid dosage used only dosage information from baseline and Weeks 24 and 52 for the calculations; changes in dosage between the different time points were not considered. Likewise, the count of glucocorticoid dosage tapers did not consider any increases in dosage, only tapers. Nonetheless, measuring changes in glucocorticoid use through two different approaches yielded comparable results that are in line with expectations of the mechanism of action of anifrolumab, which lends confidence that the results are real and meaningful. A second limitation is that no formal, empirical test of model invariance was conducted to check if the hypothesized models fit the same for each trial. However, the size of parameter estimates and comparable fit statistics, and the consistency of results across the two trials gave us confidence that the models were similar for both trial populations, and pooling the trials was justified.

To conclude, PRO outcomes in TULIP trials assessed by the hypothesized model in these analyses show that anifrolumab is associated with significant reported improvements in aspects of HRQoL, including pain, fatigue, mood, and physical function. These benefits are from the direct effect of anifrolumab treatment on disease activity and reduction in glucocorticoid dosage. These analyses support the utility of considering the effects of treatment on proximal clinical measures and more distal measures of symptoms and HRQoL when evaluating treatment benefit in terms of the patient experience in SLE.

### 11 EXAMPLE 6: ATTAINMENT OF THE LUPUS LOW DISEASE ACTIVITY STATE IN RESPONSE TO ANIFROLUMAB IN TWO PHASE 3 TRIALS

### 11.1 Background/Purpose

The Lupus Low Disease Activity State (LLDAS) is a treat-to-target (T2T) endpoint for SLE. LLDAS attainment is therefore an SLE treatment goal. In the TULIP-2 (NCT02446899) phase 3 trial, efficacy of anifrolumab, a type I IFN receptor mAb, was demonstrated using the BILAG-based Combined Lupus Assessment (BICLA) response, and these findings were supported in TULIP-1 (NCT02446912). The inventors investigated if anifrolumab treatment was associated with increased LLDAS attainment in pooled TULIP data.

### 11.2 Methods

TULIP-1/-2 were randomized, placebo-controlled, 52-week trials of IV anifrolumab (Q4W, 48 weeks) in eligible patients meeting ACR 1997 criteria for SLE with moderate to severe SLE despite standard therapy. Pooled data for the anifrolumab 300 mg and placebo groups were analysed by time point for LLDAS attainment (defined as all of the following: SLEDAI-2K<4 without major organ activity, no new disease activity, Physician's Global Assessment [0-3]<1, prednisone or equivalent<7.5 mg/day, and well-tolerated standard immunosuppressant dosing). Time to first attainment of LLDAS between treatment groups was compared using a Cox regression model, while responses were compared using logistic regression. All P-values are nominal.

### 11.3 Results

In an analysis agnostic to treatment group, 114 (13.9%) patients attained LLDAS at Week 52. Of these patients, 102 (89.5%) were also BICLA responders (analysis included the TULIP-1 anifrolumab 150 mg group). Among the 318 BICLA responders at Week 52, 102 (32.0%) attained LLDAS, indicating that LLDAS attainment was unlikely without BICLA response. LLDAS attainment increased across the 52-week trial and was attained earlier with anifrolumab 300 mg than placebo (FIG. 15) (time to first LLDAS, HR 1.76, 95% CI 1.35-2.30, P<0.001). At Week 52, 17.5% of anifrolumab-treated and 10.6% of placebo patients were in LLDAS (OR 1.8, 95% CI 1.2-2.8, P=0.008). More cumulative time (P<0.001) and percentage of time (P<0.001) was spent in LLDAS by patients receiving anifrolumab than placebo, and cumulative time in LLDAS thresholds of ≥20% (OR 1.8, 95% CI 1.2-2.7, P=0.004) and ≥50% (OR 1.9, 95% CI 1.0-3.4, P=0.035) favoured anifrolumab. Anifrolumab-treated patients were more likely to be in sustained LLDAS for ≥3 consecutive visits (18.6% vs 12.5%, OR 1.6, 95% CI 1.1-2.4, P=0.024), ≥5 consecutive visits (11.2% vs 7.7%, OR 1.5, 95% CI 0.9-2.6, P=0.106), or ≥7 consecutive visits (6.8% vs 2.7%, OR 2.6, 95% CI 1.2-5.6, P=0.014).

### 11.4 Conclusion

The inventors demonstrate that LLDAS is an attainable T2T endpoint in clinical trials. Surprisingly, although LLDAS is more stringent than BICLA, LLDAS attainment was shown to be highly coincident with BICLA response following treating with anifrolumab. Anifrolumab treatment was also surprisingly associated with earlier, more frequent, and more prolonged and sustained LLDAS attainment in SLE patients.

### 12 EXAMPLE 7: INVESTIGATING THE DISCORDANCE ON BICLA AND SRI(4) OUTCOMES IN THE TULIP-1 ANIFROLUMAB TRIAL USING DATA FROM THREE ANIFROLUMAB PHASE 2/3 TRIALS

### 12.1 Introduction

Anifrolumab is a human monoclonal antibody to the type I interferon receptor that was investigated in patients with moderate to severe SLE in the phase 2b MUSE trial and subsequently in 2 phase 3 trials, TULIP-1 and TULIP-2¹⁹⁻²¹. In these trials, responses were assessed using both the BICLA and SRI(4) composite indices¹⁹⁻²¹. An SRI(4)-based outcome was the primary endpoint in MUSE and, given the robust outcomes, it was originally selected as the primary endpoint for both TULIP-1 and TULIP-2. BICLA, a secondary endpoint that also yielded robust outcomes in MUSE and TULIP-1, was subsequently designated the primary endpoint in TULIP-2 prior to unblinding of the TULIP-2 dataset²². In TULIP-2, anifrolumab 300 mg demonstrated a statistically significant benefit compared with placebo measured by both BICLA and SRI(4) responses at Week 52; similar results were also observed in MUSE¹⁹⁻²². In TULIP-1, the effect of anifrolumab 300 mg on BICLA response was of similar magnitude to that seen in TULIP-2 and MUSE; however, the treatment difference between anifrolumab and placebo with SRI(4) did not achieve statistical significance as seen in MUSE and TULIP-2.

While BICLA and SRI(4) both evaluate clinically meaningful elements of global SLE disease activity, it is recognized that differences in their basic metric properties make it possible for a given patient's response to be inconsistently classified by these outcome measures. The BILAG-2004 index, on which the BICLA is anchored, grades each manifestation according to severity and the physician's intention to treat; it also captures incremental, clinically meaningful improvement or worsening. In contrast, the SLEDAI-2K, on which the SRI(4) is anchored, consists of dichotomous scoring (present or absent) of each manifestation independent of severity and assigns individual items a distinct number of points resulting in differential weights of the SLEDAI-2K elements. In order to be a BICLA responder, a patient must have at least partial improvement in all severe (BILAG-2004 A) or moderate (BILAG-2004 B) clinical manifestations affected at baseline, whereas to be an SRI(4) responder a patient needs to have complete resolution of enough manifestations affected at baseline to reduce the total SLEDAI-2K score by ≥4 points.

To understand the lack of a statistically significant SRI(4) treatment difference with anifrolumab in the TULIP-1 trial, the inventors investigated the concordance of BICLA and SRI(4) outcomes for each patient across the TULIP-1 and TULIP-2 trials, as well as the MUSE trial.

### 12.2 Patients and Methods

### 12.2.1 Patients and Study Design

The detailed methods for TULIP-1, TULIP-2, and MUSE have been previously reported, including study design, inclusion and exclusion criteria, and patient disposition¹⁹⁻²¹. TULIP-1, TULIP-2, and MUSE were randomized, double-blind, 52-week trials of adult patients with auto-antibody positive moderate to severe SLE despite standard therapy. TULIP-1, TULIP-2, and MUSE evaluated anifrolumab 300 mg. Anifrolumab 150 mg and 1000 mg dose groups were also evaluated in TULIP-1 and MUSE, respectively. Here we analysed data from patients who received the target dose of anifrolumab 300 mg (intravenous, every 4 weeks for 48 weeks) or placebo.

In TULIP-1 and TULIP-2, an attempt to taper oral glucocorticoids to the target of ≤7.5 mg/day (prednisone or equivalent) was mandatory between Weeks 8 and 40 for patients receiving oral glucocorticoids≥10 mg/day at baseline, and taper was considered sustained if maintained from Week 40 to Week 52. In MUSE, tapering of oral glucocorticoids was encouraged for all patients but was at the discretion of investigators.

### 12.2.2 BICLA and SRI(4) Endpoints

The TULIP-1, TULIP-2, and MUSE trials included analyses of BICLA and SRI(4) responses at Week 52. A BICLA response was defined as all of the following: reduction of all baseline BILAG-2004 A domain scores to B/C/D, and all B domain scores to C/D, and no worsening in other BILAG-2004 organ systems as defined by ≥1 new BILAG-2004 A or ≥2 new BILAG-2004 B domain scores; no increase in SLEDAI-2K score (from baseline); no increase in Physician's Global Assessment (PGA) score (≥0.3 points from baseline); no use of restricted medications beyond protocol-allowed thresholds; and no discontinuation of investigational product. An SRI(4) response was defined as all of the following: ≥4-point reduction in SLEDAI-2K; <1 new BILAG-2004 A or <2 new BILAG-2004 B organ domain scores; no increase in PGA (>0.3 points from baseline); no use of restricted medications beyond protocol-allowed thresholds; and no discontinuation of investigational product.

### 12.2.3 Assessment of Concordance on BICLA and SRI(4) Outcomes

In TULIP-1, TULIP-2, and MUSE, assessments of BICLA and SRI(4) responder status at Week 52 were performed for all patients who received anifrolumab 300 mg or placebo. Patients were grouped by concordance on BICLA and SRI(4) outcomes. Concordant subgroups included patients who were both BICLA and SRI(4) responders (“dual” responders), or patients who were both BICLA and SRI(4) nonresponders. Discordant subgroups included patients who were BICLA nonresponders and SRI(4) responders, or BICLA responders and SRI(4) nonresponders.

Concordant and discordant subgroups were further evaluated on baseline demographics and clinical characteristics, glucocorticoid taper during the trial, and responses from baseline to Week 52 across the BILAG-2004 and SLEDAI-2K organ domains. Joint count changes from baseline to Week 52 were also assessed.

### 12.2.4 Statistical Analyses

The proportion (and corresponding treatment differences, 95% confidence intervals [CIs], and nominal P-values) of patients achieving a dual BICLA and SRI(4) response in the anifrolumab group was compared with that in the placebo group using a Cochran-Mantel-Haenszel (CMH) approach controlling for stratification factors (SLEDAI-2K score at screening [<10 vs ≥10], glucocorticoid daily dose on Day 1 [<10 mg/day vs ≥10 mg/day], and type I interferon gene signature status at screening [high vs low]) (18). Percentage agreement and Cohen's Kappa were used as measures of agreement between BICLA and SRI(4) responses in each study. The percentage agreement was calculated as the number of agreement scores divided by the total number of scores (percentage agreement in MUSE was based on all patients with ≥1 BILAG-2004 A or B score at baseline). The Cohen's Kappa coefficient (K, defined as the amount by which the observed agreement exceeds that expected by chance alone,

divided by the theoretical maximum [19]) was used to evaluate the degree of concordance/reliability between the two endpoints, where: κ<0 is “no agreement,” κ=0-0.20 is “slight agreement,” κ=0.21-0.40 is “fair agreement,” κ=0.41-0.60 is “moderate agreement,” κ=0.61-0.80 is “substantial agreement,” and κ=0.81-1.0 is “perfect agreement” (20).

### 12.3 Results

### 12.3.1 Patients

The anifrolumab 300 mg and placebo groups in TULIP-1 (anifrolumab, n=180; placebo, n=184), TULIP-2 (anifrolumab, n=180; placebo, n=182), and MUSE (anifrolumab, n=99; placebo, n=102) were assessed. Patient demographics and clinical characteristics were generally balanced across treatment groups, both within the individual trials, and when comparing treatment groups across TULIP-1, TULIP-2, and MUSE (Table 12-1). The majority of patients (>91% in all groups) were female. At baseline, the mean SLEDAI-2K scores ranged from 10.7 to 11.5, and approximately half of all treatment groups had ≥1 BILAG-2004 A score (45.0%-52.5%). In total, 78.3%-86.3% of patients were receiving oral glucocorticoids at any dose, 45.6%-62.7% were receiving glucocorticoids≥10 mg/day prednisone or equivalent, and 45.1%-53.5% were receiving immunosuppressants.

TABLE 12-1
Patient baseline demographics and clinical characteristics in the MUSE, TULIP-1, and TULIP-2 trials

| | TULIP-1 | | TULIP-2 | | MUSE | |
|---|---|---|---|---|---|---|
| | | Anifrolumab | | Anifrolumab | | Anifrolumab |
| | Placebo | 300 mg | Placebo | 300 mg | Placebo | 300 mg |
| | (n = 184) | (n = 180) | (n = 182) | (n = 180) | (n = 102) | (n = 99) |
| Age, mean (SD), years | 41.0 (12.3) | 42.0 (12.0) | 41.1 (11.5) | 43.1 (12.0) | 39.3 (12.9) | 39.1 (11.9) |
| Female, n (%) | 171 (92.9) | 165 (91.7) | 170 (93.4) | 168 (93.3) | 93 (91.2) | 93 (93.9) |
| Race, n (%) | | | | | | |
| White | 137 (74.5) | 125 (69.4) | 107 (58.8) | 110 (61.1) | 41 (40.2) | 35 (35.4) |
| Black/African American | 23 (12.5) | 29 (16.1) | 25 (13.7) | 17 (9.4) | 12 (11.8) | 19 (19.2) |
| Asian | 5 (2.7) | 11 (6.1) | 30 (16.5) | 30 (16.7) | 13 (12.7) | 3 (3.0) |
| Other | 19 (10.3) | 15 (8.3) | 12 (6.6) | 15 (8.3) | 36 (35.3) | 38 (38.4) |
| Time from SLE diagnosis | 79.5 (4-503) | 88.0 (0-450) | 78.0 (6-494) | 94.5 (6-555) | 65.8 (6.9-404) | 71.4 (7.1-361) |
| to randomization, median | | | | | | |
| (range), months | | | | | | |
| IFNGS status at | | | | | | |
| screening, n (%) | | | | | | |
| High | 151 (82.1) | 148 (82.2) | 151 (83.0) | 150 (83.3) | 76 (74.5) | 75 (75.8) |
| Low | 33 (17.9) | 32 (17.8) | 31 (17.0) | 30 (16.7) | 26 (25.5) | 24 (24.2) |
| ≥1 BILAG-2004 A, n (%) | 84 (45.7) | 93 (51.7) | 95 (52.2) | 81 (45.0) | 49 (48.0) | 52 (52.5) |
| No BILAG-2004 A and ≥2 | 84 (45.7) | 79 (43.9) | 78 (42.9) | 91 (50.6) | 48 (47.1) | 41 (41.4) |
| BILAG-2004 B, n (%) | | | | | | |
| SLEDAI-2K global score, | 11.5 (3.5) | 11.3 (4.0) | 11.5 (3.9) | 11.4 (3.6) | 11.1 (4.4) | 10.7 (3.7) |
| mean (SD) | | | | | | |
| SLEDAI-2K ≥10, n (%) | 135 (73.4) | 125 (69.4) | 131 (72.0) | 129 (71.7) | 61 (59.8) | 59 (59.6) |
| PGA score, mean (SD) | 1.8 (0.4) | 1.9 (0.4) | 1.8 (0.4) | 1.7 (0.4) | 1.8 (0.4) | 1.9 (0.4) |
| CLASI activity score, | 8.1 (6.7) | 8.5 (7.3) | 7.6 (7.8) | 8.3 (7.9) | 6.7 (5.1) | 7.5 (6.3) |
| mean (SD) | | | | | | |
| Swollen joint count,^{a} mean | 7.0 (4.8) | 7.4 (5.8) | 7.4 (6.6) | 6.2 (5.7) | 8.3 (6.4) | 8.6 (6.0) |
| (SD) | | | | | | |
| Tender joint count,^{a} mean | 10.6 (7.2) | 11.7 (7.5) | 11.0 (7.9) | 9.0 (7.1) | 10.5 (7.4) | 12.2 (7.1) |
| (SD) | | | | | | |
| SDI score, mean (SD) | 0.6 (1.0) | 0.7 (1.2) | 0.5 (0.8) | 0.5 (0.9) | 0.7 (1.2) | 0.7 (1.0) |
| SLE treatments at | | | | | | |
| baseline, n (%) | | | | | | |
| GC^{b} | 153 (83.2) | 150 (83.3) | 151 (83.0) | 141 (78.3) | 88 (86.3) | 79 (79.8) |
| GC ≥10 mg/day | 102 (55.4) | 103 (57.2) | 83 (45.6) | 87 (48.3) | 64 (62.7) | 55 (55.6) |
| Antimalarials | 134 (72.8) | 124 (68.9) | 133 (73.1) | 119 (66.1) | 75 (73.5) | 76 (76.8) |
| Immunosuppressants^{c} | 91 (49.5) | 85 (47.2) | 86 (47.3) | 88 (48.9) | 46 (45.1) | 53 (53.5) |

BILAG-2004, British Isles Lupus Assessment Group-2004; CLASI, Cutaneous Lupus Erythematosus Disease Area and Severity Index; GC, glucocorticoid; IFNGS, interferon gene signature; PGA, Physician's Global Assessment; SD, standard deviation; SDI, Systemic Lupus International Collaborating Clinics/American College of Rheumatology Damage Index; SLE, systemic lupus erythematosus; SLEDAI-2K, Systemic Lupus Erythematosus Disease Activity Index 2000.
^{a}Joint count was based on 28 joints.
^{b}Prednisone or equivalent.
^{c}Immunosuppressants: azathioprine, methotrexate, mycophenolate mofetil, mycophenolic acid, and mizoribine.

### 12.3.2 BICLA and SRI(4) Concordance

The concordance in BICLA and SRI(4) responder status at Week 52 for patients in TULIP-1, TULIP-2, and MUSE is summarized in FIG. 16. Across the three trials, 85.4% (TULIP-1), 83.7% (TULIP-2), and 78.0% (MUSE) of patients had concordant BICLA and SRI(4) outcomes. The Cohen's Kappa analysis showed substantial agreement between the outcomes (TULIP-1 and TULIP-2: κ=0.7, nominal P<0.001; MUSE: κ=0.6, nominal P<0.001).

In TULIP-1, the proportions of patients who were both BICLA and SRI(4) responders (“dual” responders) were 42.2% for the anifrolumab group and 27.7% for the placebo group (FIG. 16). This treatment difference was statistically significant (14.3%; 95% CI: 4.6, 24.0; nominal P=0.004), and was consistent with those differences observed in TULIP-2 (anifrolumab 43.3%, placebo 26.4%; difference: 16.9%; 95% CI: 7.2, 26.7; nominal P<0.001) and in MUSE (anifrolumab 48.5%, placebo 20.8%, difference, 27.7%: 95% CI: 15.7, 41.5; nominal P<0.001).

### 12.3.3 BICLA and SRI(4) Discordance

Smaller proportions of patients in each study had discordant BICLA and SRI(4) outcomes (FIG. 16). In TULIP-2 and MUSE, the patterns of discordance were generally similar across the treatment groups. In TULIP-1, however, one of the key findings of this analysis was that more patients in the placebo group (n=28, 15.2%) than the anifrolumab 300 mg group (n=12, 6.7%) were BICLA nonresponders/SRI(4) responders; thus, the placebo group reduced the overall SRI(4) treatment effect for the study by −8.5 percentage points. This subgroup, comprising 11.0% (n=40) of the total TULIP-1 population, warranted further investigation.

### 12.3.4 Demographics, Clinical Characteristics, and Glucocorticoid Use in the TULIP-1 BICLA Nonresponder/SRI(4) Responder Subgroup

Patient demographics were similar in the BICLA nonresponder/SRI(4) responder subgroup compared with concordant subgroups and the overall TULIP-1 population (Table 12-2). In TULIP-1, a higher proportion of patients receiving placebo compared with anifrolumab were from Eastern Europe (38.0% vs 28.9%). This was most conspicuous in the BICLA nonresponder/SRI(4) responder subgroup, where there was a greater baseline imbalance of nearly 29 percentage points (placebo 53.6% vs anifrolumab 25.0%).

TABLE 12-2
Patient baseline demographics in TULIP-1, overall and stratified by BICLA/SRI(4) concordance

| | Concordant outcomes | | | | |
|---|---|---|---|---|---|
| | BICLA−/SRI(4)− | | BICLA+/SRI(4)+ | | Discordant outcomes |
| | | Anifrolumab | | Anifrolumab | BICLA+/SRI(4)− |
| | Placebo | 300 mg | Placebo | 300 mg | Placebo |
| | (n = 101) | (n = 83) | (n = 51) | (n = 76) | (n = 4) |
| Mean age, years (SD) | 39.1 (12.5) | 41.8 (11.2) | 43.5 (11.2) | 41.7 (12.3) | 31.8 (14.8) |
| Female, n (%) | 92 (91.1) | 75 (90.4) | 47 (92.2) | 71 (93.4) | 4 (100) |
| Race | | | | | |
| White | 72 (71.3) | 56 (67.5) | 41 (80.4) | 55 (72.4) | 2 (50.0) |
| Black/African American | 13 (12.9) | 15 (18.1) | 6 (11.8) | 11 (14.5) | 1 (25.0) |
| Asian | 3 (3.0) | 6 (7.2) | 1 (2.0) | 4 (5.3) | 0 |
| Other | 12 (11.9) | 6 (7.2) | 3 (5.9) | 6 (7.9) | 1 (25.0) |

Region

| | | | | | |
|---|---|---|---|---|---|
| Asia Pacific | 3 (3.0) | 6 (7.2) | 2 (3.9) | 4 (5.3) | 0 |
| Europe | 35 (34.7) | 22 (26.5) | 26 (51.0) | 38 (50.0) | 0 |
| Eastern Europe | 31 (30.7) | 15 (18.1) | 24 (47.1) | 33 (43.4) | 0 |
| Western Europe | 4 (4.0) | 7 (8.4) | 2 (3.9) | 5 (6.6) | 0 |
| Latin America | 13 (12.9) | 6 (7.2) | 8 (15.7) | 12 (15.8) | 1 (25.0) |
| USA/Canada | 47 (46.5) | 46 (55.4) | 13 (25.5) | 20 (26.3) | 3 (75.0) |
| Rest of world | 3 (3.0) | 3 (3.6) | 2 (3.9) | 2 (2.6) | 0 |

| | Discordant outcomes | | | | |
|---|---|---|---|---|---|
| | BICLA+/SRI(4)− | BICLA−/SRI(4)+ | | All patients | |
| | Anifrolumab | | Anifrolumab | | Anifrolumab |
| | 300 mg | Placebo | 300 mg | Placebo | 300 mg |
| | (n = 9) | (n = 28) | (n = 12) | (n = 184) | (n = 180) |
| Mean age, years (SD) | 50.4 (12.8) | 44.3 (11.8) | 38.9 (13.7) | 41.0 (12.3) | 42.0 (12.0) |
| Female, n (%) | 8 (88.9) | 28 (100) | 11 (91.7) | 171 (92.9) | 165 (91.7) |
| Race | | | | | |
| White | 6 (66.7) | 22 (78.6) | 8 (66.7) | 137 (74.5) | 125 (69.4) |
| Black/African American | 2 (22.2) | 3 (10.7) | 1 (8.3) | 23 (12.5) | 29 (16.1) |
| Asian | 1 (11.1) | 1 (3.6) | 0 | 5 (2.7) | 11 (6.1) |
| Other | 0 | 2 (7.1) | 3 (25.0) | 18 (9.8) | 15 (8.3) |

Region

| | | | | | |
|---|---|---|---|---|---|
| Asia Pacific | 1 (11.1) | 1 (3.6) | 0 | 6 (3.3) | 11 (6.1) |
| Europe | 1 (11.1) | 15 (53.6) | 3 (25.0) | 76 (41.3) | 64 (35.6) |
| Eastern Europe | 1 (11.1) | 15 (53.6) | 3 (25.0) | 70 (38.0) | 52 (28.9) |
| Western Europe | 0 | 0 | 0 | 6 (3.3) | 12 (6.7) |
| Latin America | 2 (22.2) | 3 (10.7) | 4 (33.3) | 25 (13.6) | 24 (13.3) |
| USA/Canada | 5 (55.6) | 9 (32.1) | 4 (33.3) | 72 (39.1) | 75 (41.7) |
| Rest of world | 0 | 0 | 1 (8.3) | 5 (2.7) | 6 (3.3) |

BICLA, British Isles Lupus Assessment Group-based Composite Lupus Assessment; BICLA+, a responder on BICLA; n, number of patients in analysis; SD, standard deviation; SRI(4), Systemic Lupus Erythematosus Responder Index of ≥4.
BICLA− and SRI(4)− refer to nonresponders; BICLA+ and SRI(4)+ refer to responders.

In the TULIP-1 BICLA nonresponder/SRI(4) responder subgroup, patients in the placebo group had lower baseline SLEDAI-2K scores and joint counts than anifrolumab 300 mg-treated patients (Table 12-2). The placebo group also had a lower proportion of patients with no A and ≥2 BILAG-2004 B scores. These treatment group imbalances in baseline disease activity were not observed in any of the other subgroups of TULIP-1, nor in any of the subgroups in TULIP-2 (Table 12-3, Table 12-4). Organ involvement at baseline is presented in Table 12-5.

TABLE 12-3
SLE disease activity at baseline in TULIP-1 stratified by BICLA/SRI(4) response

| | Concordant outcomes | | | | |
|---|---|---|---|---|---|
| | BICLA−/SRI(4)− | | BICLA+/SRI(4)+ | | Discordant outcomes |
| | | Anifrolumab | | Anifrolumab | BICLA+/SRI(4)− |
| Baseline disease | Placebo | 300 mg | Placebo | 300 mg | Placebo |
| characteristic | (n = 101) | (n = 83) | (n = 51) | (n = 76) | (n = 4) |

SLEDAI-2K

| | | | | | |
|---|---|---|---|---|---|
| Mean (SD) | 11.9 (3.9) | 11.6 (4.4) | 10.9 (3.0) | 10.6 (3.1) | 8.5 (1.0) |
| ≥10 points, n (%) | 76 (75.2) | 60 (72.3) | 34 (66.7) | 50 (65.8) | 1 (25.0) |

BILAG-2004

| | | | | | |
|---|---|---|---|---|---|
| Global score, mean (SD) | 18.7 (5.9) | 20.7 (6.8) | 19.6 (4.8) | 18.5 (5.2) | 16.5 (7.3) |
| ≥1 A, n (%) | 40 (39.6) | 42 (50.6) | 29 (56.9) | 39 (51.3) | 3 (75.0) |
| No A and ≥2 B, n (%) | 50 (49.5) | 38 (45.8) | 20 (39.2) | 32 (42.1) | 0 (0.0) |
| Mean PGA score (SD) | 1.84 (0.43) | 1.90 (0.40) | 1.84 (0.36) | 1.82 (0.40) | 2.08 (0.26) |
| Active joint count,^{a, b} mean (SD) | 6.8 (5.0) | 7.1 (5.8) | 5.2 (3.2) | 6.6 (5.4) | 9.5 (6.4) |
| Swollen joint count,^{a} mean (SD) | 7.4 (5.3) | 7.6 (6.0) | 6.2 (3.6) | 6.8 (5.3) | 10.0 (6.8) |
| Tender joint count,^{a} mean (SD) | 11.2 (7.8) | 12.1 (7.8) | 8.6 (5.4) | 10.3 (6.5) | 15.0 (11.2) |

| | Discordant outcomes | | | | |
|---|---|---|---|---|---|
| | BICLA+/SRI(4)− | BICLA−/SRI(4)+ | | All patients | |
| | Anifrolumab | | Anifrolumab | | Anifrolumab |
| Baseline disease | 300 mg | Placebo | 300 mg | Placebo | 300 mg |
| characteristic | (n = 9) | (n = 28) | (n = 12) | (n = 184) | (n = 180) |
| SLEDAI-2K | | | | | |
| Mean (SD) | 9.9 (1.8) | 11.4 (3.0) | 15.1 (6.1) | 11.5 (3.5) | 11.3 (4.0) |
| ≥10 points, n (%) | 5 (55.6) | 24 (85.7) | 10 (83.3) | 135 (73.4) | 125 (69.4) |
| BILAG-2004 | | | | | |
| Global score, mean (SD) | 21.6 (6.7) | 18.8 (4.6) | 20.9 (7.5) | 18.9 (5.5) | 19.8 (6.3) |
| ≥1 A, n (%) | 8 (88.9) | 12 (42.9) | 4 (33.3) | 84 (45.7) | 93 (51.7) |
| No A and ≥2 B, n (%) | 1 (11.1) | 14 (50.0) | 8 (66.7) | 84 (45.7) | 79 (43.9) |
| Mean PGA score (SD) | 2.06 (0.38) | 1.82 (0.27) | 1.78 (0.40) | 1.84 (0.38) | 1.87 (0.40) |
| Active joint count,^{a, b} mean (SD) | 9.7 (6.1) | 6.2 (4.0) | 7.9 (7.1) | 6.3 (4.5) | 7.1 (5.7) |
| Swollen joint count,^{a} mean (SD) | 9.7 (6.1) | 6.8 (4.6) | 7.9 (7.1) | 7.0 (4.8) | 7.4 (5.8) |
| Tender joint count,^{a} mean (SD) | 16.0 (8.3) | 11.1 (6.6) | 13.8 (9.7) | 10.6 (7.2) | 11.7 (7.5) |

BICLA, British Isles Lupus Assessment Group-based Composite Lupus Assessment; BILAG-2004, British Isles Lupus Assessment Group-2004; PGA, Physician's Global Assessment; SD, standard deviation; SLEDAI-2K, Systemic Lupus Erythematosus Disease Activity Index 2000; SRI(4), Systemic Lupus Erythematosus Responder Index of ≥4. BICLA− and SRI(4)− refer to nonresponders; BICLA+ and SRI(4)+ refer to responders.
^{a}The joint count was based on 28 joints.
^{b}An active joint for the SLEDAI-2K calculation was defined as a joint with tenderness and swelling.

TABLE 12-4
Disease activity and glucocorticoid use at baseline in TULIP-2 stratified by BICLA/SRI(4) response

| | Concordant outcomes | | | | Discordant outcomes | | | |
|---|---|---|---|---|---|---|---|---|
| | BICLA−/SRI(4)− | | BICLA+/SRI(4)+ | | BICLA+/SRI(4)− | | BICLA−/SRI(4)+ | |
| | | Anifrolumab | | Anifrolumab | | Anifrolumab | | Anifrolumab |
| Baseline disease | Placebo | 300 mg | Placebo | 300 mg | Placebo | 300 mg | Placebo | 300 mg |
| characteristic | (n = 105) | (n = 72) | (n = 48) | (n = 78) | (n = 9) | (n = 8) | (n = 20) | (n = 22) |
| SLEDAI-2K Mean (SD) | 11.6 (4.21) | 11.6 (3.79) | 11.2 (3.3) | 11.1 (3.5) | 8.9 (2.47) | 9.3 (1.39) | 13.1 (3.65) | 13.0 (3.83) |
| ≥10 points, n (%) | 76 (72.4) | 50 (69.4) | 33 (68.8) | 57 (73.1) | 3 (33.3) | 3 (37.5) | 19 (86.4) | 19 (95.0) |
| BILAG-2004 Global | 19.0 (5.32) | 18.9 (4.18) | 19.2 (4.49) | 18.9 (5.33) | 16.7 (4.92) | 17.9 (7.36) | 19.6 (4.49) | 17.1 (2.35) |
| score, mean (SD) | | | | | | | | |
| ≥1 A, n (%) | 54 (51.4) | 28 (38.9) | 25 (52.1) | 41 (52.6) | 7 (77.8) | 4 (50.0) | 9 (45.0) | 8 (36.4) |
| No A and ≥2 B, n (%) | 46 (43.8) | 43 (59.7) | 20 (41.7) | 31 (39.7) | 1 (11.1) | 3 (37.5) | 11 (55.0) | 14 (63.6) |
| Mean PGA score (SD) | 1.80 (0.38) | 1.71 (0.38) | 1.69 (0.46) | 1.65 (0.46) | 1.70 (0.38) | 1.81 (0.34) | 1.76 (0.33) | 1.70 (0.37) |
| Active joint count,^{a, b} mean (SD) | 7.5 (6.85) | 7.0 (6.78) | 6.9 (6.19) | 4.6 (4.37) | 7.4 (7.14) | 8.1 (6.31) | 5.3 (4.85) | 4.0 (3.31) |
| Swollen joint count,^{a} mean (SD) | 7.9 (6.93) | 7.6 (6.71) | 7.2 (6.16) | 5.1 (4.50) | 7.8 (7.50) | 8.6 (6.70) | 5.5 (4.84) | 4.9 (3.94) |
| Tender joint count,^{a} mean (SD) | 11.7 (8.25) | 10.5 (7.59) | 9.9 (7.24) | 7.6 (6.18) | 13.4 (8.25) | 15.7 (8.40) | 8.1 (5.98) | 7.0 (6.53) |

BICLA, British Isles Lupus Assessment Group-based Composite Lupus Assessment; BILAG-2004, British Isles Lupus Assessment Group-2004; PGA, Physician's Global Assessment; SD, standard deviation; SLEDAI-2K, Systemic Lupus Erythematosus Disease Activity Index 2000; SRI(4), Systemic Lupus Erythematosus Responder Index of ≥4.
BICLA− and SRI(4)− refer to nonresponders; BICLA+ and SRI(4)+ refer to responders.
^{a}Joint count was based on 28 joints.
^{b}Prednisone or equivalent.

TABLE 12-5
Baseline organ involvement in TULIP-1, overall and stratified by BICLA/SRI(4) response

| | Concordant outcomes | | | | Discordant outcomes | | | | | |
|---|---|---|---|---|---|---|---|---|---|---|
| | BICLA−/SRI(4)− | | BICLA+/SRI(4)+ | | BICLA+/SRI(4)− | | BICLA−/SRI(4)+ | | All patients | |
| | | Anifrolumab | | Anifrolumab | | Anifrolumab | | Anifrolumab | | Anifrolumab |
| Baseline disease | Placebo | 300 mg | Placebo | 300 mg | Placebo | 300 mg | Placebo | 300 mg | Placebo | 300 mg |
| characteristic | (n = 101) | (n = 83) | (n = 51) | (n = 76) | (n = 4) | (n = 9) | (n = 28) | (n = 12) | (n = 184) | (n = 180) |

SLEDAI-2K organ domain involvement^{a}

| | | | | | | | | | | |
|---|---|---|---|---|---|---|---|---|---|---|
| Central nervous | 1 (1.0) | 1 (1.2) | 0 | 0 | 0 | 0 | 0 | 1 (8.3) | 1 (0.5) | 2 (1.1) |
| system | | | | | | | | | | |
| Vascular | 10 (9.9) | 9 (10.8) | 4 (7.8) | 7 (9.2) | 0 | 0 | 4 (14.3) | 2 (16.7) | 18 (9.8) | 18 (10.0) |
| Musculoskeletal | 98 (97.0) | 78 (94.0) | 49 (96.1) | 70 (92.1) | 3 (75.0) | 8 (88.9) | 28 (100) | 11 (91.7) | 178 (96.7) | 167 (92.8) |
| Renal | 11 (10.9) | 10 (12.0) | 3 (5.9) | 2 (2.6) | 0 | 0 | 2 (7.1) | 3 (25.0) | 16 (8.7) | 15 (8.3) |
| Mucocutaneous | 96 (95.0) | 80 (96.4) | 50 (98.0) | 73 (96.1) | 4 (100) | 9 (100) | 28 (100) | 12 (100) | 178 (96.7) | 174 (96.7) |
| Cardiovascular | 9 (8.9) | 9 (10.8) | 1 (2.0) | 4 (5.3) | 0 | 1 (11.1) | 0 | 2 (16.7) | 10 (5.4) | 16 (8.9) |
| Immunology | 68 (67.3) | 47 (56.6) | 32 (62.7) | 49 (64.5) | 1 (25.0) | 5 (55.6) | 17 (60.7) | 9 (75.0) | 118 (64.1) | 110 (61.1) |
| Hematological | 17 (16.8) | 12 (14.5) | 5 (9.8) | 6 (7.9) | 0 | 1 (11.1) | 0 | 1 (8.3) | 22 (12.0) | 20 (11.1) |
| and fever | | | | | | | | | | |

BILAG-2004 organ domain involvement^{a}

| | | | | | | | | | | |
|---|---|---|---|---|---|---|---|---|---|---|
| Constitutional | 8 (7.9) | 7 (8.4) | 2 (3.9) | 3 (3.9) | 0 | 0 | 1 (3.6) | 0 | 11 (6.0) | 10 (5.6) |
| Mucocutaneous | 82 (81.2) | 72 (86.7) | 45 (88.2) | 67 (88.1) | 3 (75.0) | 9 (100) | 28 (100) | 12 (100) | 158 (85.9) | 160 (88.9) |
| Neuropsychiatric | 3 (3.0) | 7 (8.4) | 0 | 0 | 0 | 0 | 0 | 1 (8.3) | 3 (1.6) | 8 (4.4) |
| Musculoskeletal | 91 (90.1) | 76 (91.6) | 47 (92.2) | 66 (86.8) | 3 (75.0) | 7 (77.8) | 26 (92.9) | 10 (83.3) | 167 (90.8) | 159 (88.3) |
| Cardiorespiratory | 7 (6.9) | 8 (9.6) | 1 (2.0) | 5 (6.6) | 0 | 1 (11.1) | 1 (3.6) | 2 (16.7) | 9 (4.9) | 16 (8.9) |
| Gastrointestinal | 0 | 0 | 0 | 0 | 0 | 0 | 1 (3.6) | 0 | 1 (0.5) | 0 |
| Ophthalmic | 0 | 0 | 0 | 1 (1.3) | 0 | 0 | 0 | 0 | 0 | 1 (0.6) |
| Renal | 10 (9.9) | 10 (12.0) | 4 (7.8) | 2 (2.6) | 0 | 0 | 1 (3.6) | 3 (25.0) | 15 (8.2) | 15 (8.3) |
| Hematological | 0 | 1 (1.2) | 1 (2.0) | 0 | 0 | 0 | 0 | 0 | 1 (0.6) | 1 (0.6) |

BICLA, British Isles Lupus Assessment Group-based Combined Lupus Assessment; BILAG-2004, British Isles Lupus Assessment Group-2004; SLEDAI-2K, Systemic Lupus Erythematosus Disease Activity Index 2000; SRI(4), Systemic Lupus Erythematosus Responder Index of ≥4.
BICLA− and SRI(4)− refer to nonresponders; BICLA+ and SRI(4)+ refer to responders.
^{a}Involvement defined as an A or B score at baseline.

In the TULIP-1 BICLA nonresponder/SRI(4) responder subgroup, placebo and anifrolumab groups did not differ in the proportions of patients receiving oral glucocorticoids at any dose or ≥10 mg/day at baseline. However, the mean daily glucocorticoid dose at baseline was lower in the placebo group than in the anifrolumab group, although standard deviations were large (mean [SD], 9.5 [5.8] mg/day vs 11.6 [5.8] mg/day) (Table 12-6). The proportion of patients achieving glucocorticoid taper to ≤7.5 mg/day was also lower in the placebo group than in the anifrolumab 300 mg group (53.3% vs 71.4%) (FIG. 17). As with disease activity, these imbalances between the treatment groups in the degree of glucocorticoid use during the study were not observed in the concordant TULIP-1 subgroups.

TABLE 12-6
SLE-related treatments at baseline in TULIP-1, stratified by BICLA/SRI(4) response

| | Concordant outcomes | | | | |
|---|---|---|---|---|---|
| | BICLA−/SRI(4)− | | BICLA+/SRI(4)+ | | Discordant outcomes |
| | | Anifrolumab | | Anifrolumab | BICLA+/SRI(4)− |
| | Placebo | 300 mg | Placebo | 300 mg | Placebo |
| | (n = 101) | (n = 83) | (n = 51) | (n = 76) | (n = 4) |
| Oral GC, n (%) | 86 (85.1) | 68 (81.9) | 39 (76.5) | 65 (85.5) | 4 (100) |
| ≥10 mg/day, n (%) | 57 (56.4) | 47 (56.6) | 26 (51.0) | 44 (57.9) | 4 (100) |
| Dose, mean (SD), mg/day^{c} | 12.3 (7.7) | 14.6 (16.3) | 11.9 (8.3) | 11.1 (6.3) | 17.5 (9.6) |
| Antimalarials, n (%) | 76 (75.2) | 54 (65.1) | 40 (78.4) | 54 (71.1) | 2 (50.0) |
| Immunosuppressants, n (%) | 54 (53.5) | 43 (51.8) | 24 (47.1) | 30 (39.5) | 1 (25.0) |
| NSAIDs, n (%) | 20 (19.8) | 16 (19.3) | 8 (15.7) | 9 (11.8) | 1 (25.0) |

| | Discordant outcomes | | | | |
|---|---|---|---|---|---|
| | BICLA+/SRI(4)− | BICLA−/SRI(4)+ | | All patients | |
| | Anifrolumab | | Anifrolumab | | Anifrolumab |
| | 300 mg | Placebo | 300 mg | Placebo | 300 mg |
| | (n = 9) | (n = 28) | (n = 12) | (n = 184) | (n = 180) |
| Oral GC, n (%) | 7 (77.8) | 24 (85.7) | 10 (83.3) | 153 (83.2) | 150 (83.3) |
| ≥10 mg/day, n (%) | 5 (55.6) | 15 (53.6) | 7 (58.3) | 102 (55.4) | 103 (57.2) |
| Dose, mean (SD), mg/day^{c} | 12.9 (7.0) | 9.5 (5.8) | 11.6 (5.8) | 11.9 (7.7) | 12.8 (12.0) |
| Antimalarials, n (%) | 7 (77.8) | 16 (57.1) | 9 (75.0) | 134 (72.8) | 124 (68.9) |
| Immunosuppressants, n (%) | 6 (66.7) | 12 (42.9) | 6 (50.0) | 91 (49.5) | 85 (47.2) |
| NSAIDs, n (%) | 2 (22.2) | 6 (21.4) | 4 (33.3) | 35 (19.0) | 31 (17.2) |

BICLA, British Isles Lupus Assessment Group-based Composite Lupus Assessment; GC, glucocorticoid; NSAID, nonsteroidal anti-inflammatory drug; SD, standard deviation; SRI(4), Systemic Lupus Erythematosus Responder Index of ≥4.
^{a}Oral GC includes prednisone or equivalent.
BICLA− and SRI(4)− refer to nonresponders; BICLA+ and SRI(4)+ refer to responders.

### 12.3.5 SRI(4) Response Characteristics in the TULIP-1 BICLA Nonresponder/SRI(4) Responder Subgroup

In the TULIP-1 BICLA nonresponder/SRI(4) responder subgroup, the majority (22/28, 78.6%) of patients in the placebo group attained the 4-point reduction in SLEDAI-2K required for an SRI(4) response as a result of their arthritis response (FIG. 18A, Table 12-7); of note, 25.0% (7/28) of patients in the placebo group attained resolution only in the arthritis domain, whereas no patients in the anifrolumab group had responses solely restricted to the arthritis domain (Table 12-7). In the anifrolumab 300 mg group, there was far greater variation in domain improvements leading to SRI(4) responses.

TABLE 12-7
Reasons for SRI(4) response at Week 52 in TULIP-1
among BICLA nonresponders/SRI(4) responders

| | BICLA nonresponders/SRI(4) | | | |
|---|---|---|---|---|
| | responder in TULIP-1 | | | |
| SLEDAI-2K | Placebo | | Anifrolumab 300 mg | |
| domain, n (%) | (n = 28) | | (n = 12) | |
| Arthritis | 22 | (78.6) | 6 | (50.0) |
| Arthritis only | 7 | (25.0) | 0 | |
| Arthritis + other items | 15 | (53.6) | 6 | (50.0) |
| Rash | 1 | (3.6) | 2 | (16.7) |
| Rash + other items | 1 | (3.6) | 2 | (16.7) |
| Proteinuria | 2 | (7.1) | 2 | (16.7) |
| Proteinuria only | 1 | (3.6) | 0 | |
| Proteinuria + other items | 1 | (3.6) | 2 | (16.7) |
| Mucosal ulcers + anti-dsDNA | 0 | | 1 | (8.3) |
| Mucosal ulcers + low | 1 | (3.6) | 0 | |
| complement | | | | |
| Alopecia + low complement + | 0 | | 1 | (8.3) |
| leukopenia | | | | |
| Vasculitis + low complement | 1 | (3.6) | 0 | |
| Anti-dsDNA antibodies + low | 1 | (3.6) | 0 | |
| complement | | | | |

anti-dsDNA, anti-double-stranded DNA; BICLA, British Isles Lupus Assessment Group-based Composite Lupus Assessment; SLEDAI-2K, Systemic Lupus Erythematosus Disease Activity Index 2000; SRI(4), Systemic Lupus Erythematosus Responder Index of ≥4.

In light of the above findings, the inventors determined the baseline joint counts of patients who had a SLEDAI-2K arthritis response. Among the 22/28 BICLA nonresponders/SRI(4) responders in the placebo group who had SLEDAI-2K arthritis responses, 11 patients (50.0%) had <6 swollen and <6 tender joints at baseline compared with 2/6 (33.3%) anifrolumab-treated patients in this subgroup (FIG. 19). In addition, of the 22 patients in the placebo group who had SLEDAI-2K arthritis response, 12 were receiving ≥10 mg/day glucocorticoid at baseline, of whom 5 (41.6%) were unable to taper glucocorticoids to $7.5 mg/day. In contrast, of the 6 anifrolumab-treated patients who had SLEDAI-2K arthritis resolution, none of the 4 patients who were receiving ≥10 mg/day glucocorticoid at baseline failed to taper their glucocorticoids to ≤7.5 mg/day.

### 12.3.6 Reasons for BICLA Nonresponse in the TULIP-1 BICLA Nonresponder/SRI(4) Responder Subgroup

In the TULIP-1 BICLA nonresponder/SRI(4) responder subgroup, patients achieving a response on items resulting in a 4-point reduction in SLEDAI-2K also improved in the same organ domains on BILAG-2004. However, patients in this subgroup were BICLA nonresponders because a BICLA response required improvement in all baseline BILAG-2004 manifestations that were moderate or severe at study entry, and these patients had other unresolved organ involvement at Week 52. The most common reason for a BICLA nonresponse in the TULIP-1 BICLA nonresponder/SRI(4) responder subgroup was a lack of improvement in BILAG-2004 rash in both the anifrolumab (8/12, 66.7%) and placebo groups (24/28, 85.7%) (FIG. 18B, Table 12-8).

TABLE 12-8
Reasons for BICLA nonresponse at Week 52 in TULIP-1
among BICLA nonresponders/SRI(4) responders

| | BICLA nonresponders/SRI(4) | | | |
|---|---|---|---|---|
| BICLA nonresponse (no | responders in TULIP-1 | | | |
| improvement in BILAG-2004 | Placebo | | Anifrolumab 300 mg | |
| A or B items), n (%) | (n = 28) | | (n = 12) | |
| Arthritis | 2 | (7.1) | 2 | (16.7) |
| Arthritis only | 2 | (7.1) | 2 | (16.7) |
| Arthritis + other items | 0 | | 0 | |
| Rash | 24 | (85.7) | 8 | (66.7) |
| Rash only | 15 | (53.6) | 5 | (41.7) |
| Rash + other items | 9 | (32.1) | 3 | (25.0) |

| | | | |
|---|---|---|---|
| Cognitive dysfunction | 0 | 1 | (8.3) |
| Pleurisy/pericarditis | 0 | 1 | (8.3) |

| | | | |
|---|---|---|---|
| Lupus hepatitis | 1 | (3.6) | 0 |
| Interstitial alveolitis/pneumonitis | 1 | (3.6) | 0 |

BICLA, British Isles Lupus Assessment Group-based Composite Lupus Assessment; BILAG-2004, British Isles Lupus Assessment Group-2004; SRI(4), Systemic Lupus Erythematosus Responder Index of ≥4.

Overall, in this subgroup, the combination of BICLA nonresponse due to rash and SRI(4) response due to arthritis occurred in 20 (71.4%) placebo patients and 5 (41.7%) anifrolumab-treated patients (Table 12-9).

TABLE 12-9
Overview of reasons for a BICLA nonresponse/SRI(4)
response at Week 52 in TULIP-1 among BICLA
nonresponders/SRI(4) responders

| | SRI(4) responders/BICLA | | |
|---|---|---|---|
| | nonresponders in TULIP-1 | | |
| BICLA nonresponse/ | Placebo | | Anifrolumab 300 mg |
| SRI response, n (%) | (n = 28) | | (n = 12) |
| BICLA nonresponse due to rash/ | 20 | (71.4) | 5 (41.7) |
| SRI response due to arthritis | | | |
| BICLA nonresponse due to rash/ | 4 | (14.3) | 3 (25.0) |
| SRI response not due to arthritis | | | |
| BICLA nonresponse on arthritis/ | 2 | (7.1) | 2 (16.7) |
| SRI response due to any reason | | | |
| Other^{a} BICLA nonresponse/ | 2 | (7.1) | 2 (16.7) |
| SRI response due to any reason | | | |

BICLA, British Isles Lupus Assessment Group-based Composite Lupus Assessment; SRI(4), Systemic Lupus Erythematosus Responder Index of ≥4.
^{a}Nonresponse due to a clinical manifestation other than rash or arthritis.

### 12.4 Discussion

In the anifrolumab clinical development program for the treatment of patients with SLE, consistent BICLA and SRI(4) outcomes in favour of anifrolumab were observed in the TULIP-2 and MUSE trials, but not in the TULIP-1 trial. Here, the inventors studied BICLA and SRI(4) outcomes at an individual patient level across all three trials and found a high level of concordance between both composite endpoints. The proportion of patients in TULIP-1 who met the stringent “dual responder” criteria (i.e., met both BICLA and SRI [4] response definitions) favoured anifrolumab (treatment difference, 14.3%), and was similar to the effect size for “dual responders” seen in TULIP-2 (treatment difference 16.9%), supporting the beneficial effect of anifrolumab on global disease activity in patients with SLE.

Discordant outcomes were observed in small proportions of patients in all three trials. In contrast to the TULIP-2 and MUSE trials, where the proportions of discordant patients were generally similar in the anifrolumab and placebo treatment groups, BICLA nonresponders/SRI(4) responders were more frequently observed in the placebo group in TULIP-1. This subgroup of patients was likely responsible for the lack of significant SRI(4) treatment differences in TULIP-1.

In the BICLA nonresponder/SRI(4) responder subgroup in TULIP-1, the primary reason for SRI(4) response was SLEDAI-2K resolution of arthritis (with a weight of 4 points), which by itself was sufficient to achieve an SRI(4) response. This single domain response is not sufficient to achieve a BICLA response unless all other baseline organ domain activity also improves. Patients in the BICLA nonresponder/SRI(4) responder subgroup who achieved a SLEDAI-2K response also improved in the equivalent BILAG-2004 organ domains. However, since a BICLA response requires improvement in all moderate to severe baseline manifestations, patients with additional clinical manifestations, predominantly rash, who did not improve enough to satisfy criteria for a BICLA response were classified as SRI(4) responders but BICLA nonresponders.

As SRI(4) response can be driven by arthritis resolution alone, we assessed how many active joints resolved to attain an SRI(4) response in the BICLA nonresponder/SRI(4) responder group in TULIP-1, and found that baseline joint scores tended to be lower in the placebo group than in the anifrolumab group. Therefore, SLEDAI-2K arthritis responses could more easily be achieved in the placebo group than the anifrolumab group. Another key observation in this TULIP-1 subgroup was the lower proportion of patients in the placebo group versus the anifrolumab group who achieved glucocorticoid taper to ≤7.5 mg/day. It is therefore possible that the lower degree of glucocorticoid tapering (resulting in greater glucocorticoid exposure), coupled with a higher proportion of patients with ≤6 active joints at baseline, may have inflated the proportion of patients with an SRI(4) response in the placebo group compared with the anifrolumab group.

Variations in glucocorticoid tapering may reflect regional differences in the use of standard therapies. The discordant subgroup in TULIP-1 had a higher proportion of patients from Eastern versus Western Europe. In a previous international inception cohort study, there was significant between-centre variation in glucocorticoid use, even after adjustment for patient factors known to influence glucocorticoid dosage requirements. Our observations from TULIP-1 support the notion that physicians vary in glucocorticoid prescribing behaviours, which if not accounted for, can create an imbalance in trial outcomes.

The inventor's analyses highlight the potential for inconsistent BICLA and SRI(4) outcomes among individual patients. In a phase 2 placebo-controlled study of epratuzumab, the SRI(4) response rate in the placebo group was higher than that in the active groups, and the BICLA response rate was lower than the SRI(4) response rate in all treatment groups⁵². Similarly, despite similar SRI(4) and BICLA placebo response rates (33%) in the phase 2 trial of ustekinumab, the SRI(4) ustekinumab response rate was 62%, whereas the BICLA response rate was 35% 53. Notably, two reviews comparing BICLA and SRI(4) as measures of global SLE disease activity concluded that, while both are viable tools for use as primary endpoints in SLE studies, differences in the populations under study and in study designs can impact the outcomes of each measure^{54,55}. The TULIP-1 results presented herein suggest that BICLA/SRI(4) discordance is more likely to occur in patients with two or more active organ domains if the weighting of key active organs (i.e., arthritis; worth 4 SLEDAI-2K points) allows an SRI(4) response to be met by a single organ-domain resolution alone.

This secondary analysis of TULIP-1 data provides some important lessons for future SLE trial design. Variations in the number of active organ domains and joint counts at baseline, glucocorticoid prescribing/tapering practices, and/or regions of recruitment into trials may all increase the risk of discordant BICLA and SRI(4) outcomes. Imbalances in these demographic and clinical factors at baseline potentially jeopardize the primary outcome; therefore, every effort should be made to ensure adequate balancing of these factors at entry into, and during the running of, clinical trials. In addition, setting a minimum threshold for active joint counts within the arthritis domain, as well as thresholds for other manifestations such as rash, oral ulcers, and alopecia, may improve the stringency of an endpoint such as SRI(4), which can be confounded by improvement in one or two highly-weighted organ domains in patients with low disease activity at baseline.

To conclude, in individual patient-level analyses, the inventors found that the majority of patients in the TULIP-1, TULIP-2, and MUSE trials of anifrolumab had concordant outcomes on BICLA and SRI(4). The proportion of patients who met the stringent BICLA and SRI(4) “dual-responder” criteria favoured anifrolumab over placebo in all three trials. A discordant BICLA nonresponder/SRI(4) responder subgroup was identified in all three trials, and this subgroup was over-represented in the placebo group of TULIP-1. Discordance was largely driven by the sensitivity of SRI(4) to single organ (arthritis) improvement coupled with the stringent requirement of BICLA for improvement in all domains with baseline BILAG-2004 A or B scores. Discordant placebo-treated patients showed regional recruitment variation, tended to have lower baseline disease activity and joint counts, and were less likely to taper glucocorticoids. Careful attention to baseline factors and minimizing variation in glucocorticoid tapering will be essential in future SLE clinical trials.

### 13 EXAMPLE 8: INJECTION DEVICE

Anifrolumab is administered by an injection device [1] [9] such as a prefilled syringe (PFS) (FIG. 20A) or an autoinjector (AI) (FIG. 20B).

### 13.1 Autoinjector

Anifrolumab may be administered by an autoinjector [1]. The autoinjector is shown in exploded view (FIG. 21A) and in an assembled form (FIG. 21B, FIG. 21C). A label [4] is wrapped around and attached to the autoinjector [1] (C). The autoinjector has an autoinjector housing [3], cap and cap remover [2] and drive unit [5]. The liquid anifrolumab formulation unit dose [6] is contained in the autoinjector housing [3]. The unit dose [6] can be viewed through the viewing window [7].

### 13.2 Accessorized Pre-Frilled Syringe

Anifrolumab may be administered by accessorized pre-filled syringe (APFS) [8]. The APFS [8] includes the unit dose of anifrolumab [6] contained in a primary container [9] shown in an assembled state in FIG. 22A and in an exploded view in FIG. 22B. The primary container [9] has a plunger stopper [16]. The primary container has a nominal fill volume of 0.8 ml but may contain slightly more than 0.8 ml. The remainder of the space in the primary container [9] is taken up by an air bubble [18]. The air bubble may have a size of 3-5 mm, optionally, 4 mm. The primary container [9] has a defined stopper position [19].

The accessorized pre-filled syringe (APFS) primary container [9] is provided in a PFS assembly [8] including a needle guard [12], a finger flange and a plunger rod (FIG. 22C, FIG. 22D). A label [14] is provided with the primary container [9] in the PFS assembly [8]. The label is wrapped around the syringe [9] in the label placement position [15].

### 13.3 Packaging

The injection device [1] [8] is provided in a kit (FIG. 23). A label [4] is provided with the APFS or autoinjector in the packaging. The label includes instruction for the use of the injection device [1], [8]. The packaging includes a tamper seal.

### REFERENCES

All publications mentioned in the specification are herein incorporated by reference.
(1) Pons-Estel, G. J.; Alarcón, G. S.; Scofield, L.; Reinlib, L.; Cooper, G. S. Understanding the Epidemiology and Progression of Systemic Lupus Erythematosus. Semin Arthritis Rheum 2010, 39 (4), 257-268. https://doi.org/10.1016/j.semarthrit.2008.10.007.
(2) McCauliffe, D. P. Cutaneous Lupus Erythematosus. Semin Cutan Med Surg 2001, 20 (1), 14-26. https://doi.org/10.1053/sder.2001.23091.
(3) Uva, L.; Miguel, D.; Pinheiro, C.; Freitas, J. P.; Marques Gomes, M.; Filipe, P. Cutaneous Manifestations of Systemic Lupus Erythematosus. Autoimmune Dis 2012, 2012, 834291. https://doi.org/10.1155/2012/834291.
(4) Zayat, A. S.; Md Yusof, M. Y.; Wakefield, R. J.; Conaghan, P. G.; Emery, P.; Vital, E. M. The Role of Ultrasound in Assessing Musculoskeletal Symptoms of Systemic Lupus Erythematosus: A Systematic Literature Review. Rheumatology (Oxford) 2016, 55 (3), 485-494. https://doi.org/10.1093/rheumatology/kev343.
(5) Sa, C.; E, A.; A, R.; D, I. Damage and mortality in a group of British patients with systemic lupus erythematosus followed up for over 10 years https://pubmed.ncbi.nlm.nih.gov/19359343/(accessed 2021 Feb. 8). https://doi.org/10.1093/rheumatology/kep062.
(6) Murimi-Worstell, I. B.; Lin, D. H.; Nab, H.; Kan, H. J.; Onasanya, O.; Tierce, J. C.; Wang, X.; Desta, B.; Alexander, G. C.; Hammond, E. R. Association between Organ Damage and Mortality in Systemic Lupus Erythematosus: A Systematic Review and Meta-Analysis. BMJ Open 2020, 10 (5), e031850. https://doi.org/10.1136/bmjopen-2019-031850.
(7) Doria, A.; Briani, C. Lupus: Improving Long-Term Prognosis. Lupus 2008, 17 (3), 166-170. https://doi.org/10.1177/0961203307087612.
(8) Petri, M. Long-Term Outcomes in Lupus. Am J Manag Care 2001, 7 (16 Suppl), S480-485.
(9) Zonana-Nacach, A.; Barr, S. G.; Magder, L. S.; Petri, M. Damage in Systemic Lupus Erythematosus and Its Association with Corticosteroids. Arthritis Rheum 2000, 43 (8), 1801-1808. https://doi.org/10.1002/1529-0131(200008)43:8<1801:AID-ANR16>3.0.CO; 2-O.
(10) Urowitz, M. B.; Bookman, A. A.; Koehler, B. E.; Gordon, D. A.; Smythe, H. A.; Ogryzlo, M. A. The Bimodal Mortality Pattern of Systemic Lupus Erythematosus. Am J Med 1976, 60 (2), 221-225. https://doi.org/10.1016/0002-9343(76)90431-9.
(11) Patel, D. D.; Antoni, C.; Freedman, S. J.; Levesque, M. C.; Sundy, J. S. Phase 2 to Phase 3 Clinical Trial Transitions: Reasons for Success and Failure in Immunologic Diseases. Journal of Allergy and Clinical Immunology 2017, 140 (3), 685-687. https://doi.org/10.1016/j.jaci.2017.04.029.
(12) Dowden, H.; Munro, J. Trends in Clinical Success Rates and Therapeutic Focus. Nature Reviews Drug Discovery 2019, 18 (7), 495-496. https://doi.org/10.1038/d41573-019-00074-z.
(13) Eisenberg, R. WHY CAN'T WE FIND A NEW TREATMENT FOR SLE? J Autoimmun 2009, 32 (3-4), 223-230. https://doi.org/10.1016/j.jaut.2009.02.006.
(14) Gomez, A.; Qiu, V.; Cederlund, A.; Borg, A.; Lindblom, J.; Emamikia, S.; Enman, Y.; Lampa, J.; Parodis, I. Adverse Health-Related Quality of Life Outcome Despite Adequate Clinical Response to Treatment in Systemic Lupus Erythematosus. Frontiers in Medicine 2021, 8, 471. https://doi.org/10.3389/fmed.2021.651249.
(15) Blair, H. A.; Duggan, S. T. Belimumab: A Review in Systemic Lupus Erythematosus. Drugs 2018, 78 (3), 355-366. https://doi.org/10.1007/s40265-018-0872-z.
(16) Navarra, S. V.; Guzman, R. M.; Gallacher, A. E.; Hall, S.; Levy, R. A.; Jimenez, R. E.; Li, E. K.-M.; Thomas, M.; Kim, H.-Y.; Leon, M. G.; Tanasescu, C.; Nasonov, E.; Lan, J.-L.; Pineda, L.; Zhong, Z. J.; Freimuth, W.; Petri, M. A.; BLISS-52 Study Group. Efficacy and Safety of Belimumab in Patients with Active Systemic Lupus Erythematosus: A Randomised, Placebo-Controlled, Phase 3 Trial. Lancet 2011, 377 (9767), 721-731. https://doi.org/10.1016/S0140-6736 (10) 61354-2.
(17) Furie, R.; Petri, M.; Zamani, O.; Cervera, R.; Wallace, D. J.; Tegzová, D.; Sanchez-Guerrero, J.; Schwarting, A.; Merrill, J. T.; Chatham, W. W.; Stohl, W.; Ginzler, E. M.; Hough, D. R.; Zhong, Z. J.; Freimuth, W.; van Vollenhoven, R. F.; BLISS-76 Study Group. A Phase III, Randomized, Placebo-Controlled Study of Belimumab, a Monoclonal Antibody That Inhibits B Lymphocyte Stimulator, in Patients with Systemic Lupus Erythematosus. Arthritis Rheum 2011, 63 (12), 3918-3930. https://doi.org/10.1002/art.30613.
(18) van Vollenhoven, R. F.; Petri, M.; Wallace, D. J.; Roth, D. A.; Molta, C. T.; Hammer, A. E.; Tang, Y.; Thompson, A. Cumulative Corticosteroid Dose Over Fifty-Two Weeks in Patients With Systemic Lupus Erythematosus: Pooled Analyses From the Phase III Belimumab Trials. Arthritis Rheumatol 2016, 68 (9), 2184-2192. https://doi.org/10.1002/art.39682.
(19) Morand, E. F.; Furie, R.; Tanaka, Y.; Bruce, I. N.; Askanase, A. D.; Richez, C.; Bae, S.-C.; Brohawn, P. Z.; Pineda, L.; Berglind, A.; Tummala, R. Trial of Anifrolumab in Active Systemic Lupus Erythematosus. New England Journal of Medicine 2020, 382 (3), 211-221. https://doi.org/10.1056/NEJMoa1912196.
(20) Furie, R.; Khamashta, M.; Merrill, J. T.; Werth, V. P.; Kalunian, K.; Brohawn, P.; Illei, G. G.; Drappa, J.; Wang, L.; Yoo, S.; Investigators, for the C. S. Anifrolumab, an Anti-Interferon-α Receptor Monoclonal Antibody, in Moderate-to-Severe Systemic Lupus Erythematosus. Arthritis & Rheumatology (Hoboken, N.j.) 2017, 69 (2), 376. https://doi.org/10.1002/art.39962.
(21) Furie, R. A.; Morand, E. F.; Bruce, I. N.; Manzi, S.; Kalunian, K. C.; Vital, E. M.; Ford, T. L.; Gupta, R.; Hiepe, F.; Santiago, M.; Brohawn, P. Z.; Berglind, A.; Tummala, R. Type I Interferon Inhibitor Anifrolumab in Active Systemic Lupus Erythematosus (TULIP-1): A Randomised, Controlled, Phase 3 Trial. The Lancet Rheumatology 2019, 1 (4), e208-e219. https://doi.org/10.1016/S2665-9913 (19) 30076-1.
(22) Tanaka, Y.; Tummala, R. Anifrolumab, a Monoclonal Antibody to the Type I Interferon Receptor Subunit 1, for the Treatment of Systemic Lupus Erythematosus: An Overview from Clinical Trials. Modern Rheumatology 2020, 0 (0), 1-12. https://doi.org/10.1080/14397595.2020.1812201.
(23) Golder, V.; Tsang-A-Sjoe, M. W. P. Treatment Targets in SLE: Remission and Low Disease Activity State. Rheumatology 2020, 59 (Supplement_5), v19-v28. https://doi.org/10.1093/rheumatology/keaa420.
(24) Parodis, I.; Emamikia, S.; Gomez, A.; Gunnarsson, I.; van Vollenhoven, R. F.; Chatzidionysiou, K. Clinical SLEDAI-2K Zero May Be a Pragmatic Outcome Measure in SLE Studies. Expert Opinion on Biological Therapy 2019, 19 (2), 157-168. https://doi.org/10.1080/14712598.2019.1561856.
(25) Waldheim, E.; Ajeganova, S.; Bergman, S.; Frostegård, J.; Welin, E. Variation in Pain Related to Systemic Lupus Erythematosus (SLE): A 7-Year Follow-up Study. Clin Rheumatol 2018, 37 (7), 1825-1834. https://doi.org/10.1007/s10067-018-4079-1.
(26) Greco, C. M.; Rudy, T. E.; Manzi, S. Adaptation to Chronic Pain in Systemic Lupus Erythematosus: Applicability of the Multidimensional Pain Inventory. Pain Med 2003, 4 (1), 39-50. https://doi.org/10.1046/j.1526-4637.2003.03001.x.
(27) Tummala, R.; Rouse, T.; Berglind, A.; Santiago, L. Safety, Tolerability and Pharmacokinetics of Subcutaneous and Intravenous Anifrolumab in Healthy Volunteers. Lupus Sci Med 2018, 5 (1), e000252. https://doi.org/10.1136/lupus-2017-000252.
(28) Ferguson, G. T.; Mansur, A. H.; Jacobs, J. S.; Hebert, J.; Clawson, C.; Tao, W.; Wu, Y.; Goldman, M. Assessment of an Accessorized Pre-Filled Syringe for Home-Administered Benralizumab in Severe Asthma. J Asthma Allergy 2018, 11, 63-72. https://doi.org/10.2147/JAA.S157762.
(29) Albrecht, J.; Taylor, L.; Berlin, J. A.; Dulay, S.; Ang, G.; Fakharzadeh, S.; Kantor, J.; Kim, E.; Militello, G.; McGinnis, K.; Richardson, S.; Treat, J.; Vittorio, C.; Van Voorhees, A.; Werth, V. P. The CLASI (Cutaneous Lupus Erythematosus Disease Area and Severity Index): An Outcome Instrument for Cutaneous Lupus Erythematosus. J Invest Dermatol 2005, 125 (5), 889-894. https://doi.org/10.1111/j.0022-202X.2005.23889.x.
(30) Klein, R. S.; Morganroth, P. A.; Werth, V. P. Cutaneous Lupus and the CLASI Instrument. Rheum Dis Clin North Am 2010, 36 (1), 33-51. https://doi.org/10.1016/j.rdc.2009.12.001.
(31) Franklyn, K.; Lau, C. S.; Navarra, S. V.; Louthrenoo, W.; Lateef, A.; Hamijoyo, L.; Wahono, C. S.; Chen, S. L.; Jin, O.; Morton, S.; Hoi, A.; Huq, M.; Nikpour, M.; Morand, E. F. Definition and Initial Validation of a Lupus Low Disease Activity State (LLDAS). Annals of the Rheumatic Diseases 2016, 75 (9), 1615-1621. https://doi.org/10.1136/annrheumdis-2015-207726.
(32) Yellen, S. B.; Cella, D. F.; Webster, K.; Blendowski, C.; Kaplan, E. Measuring Fatigue and Other Anemia-Related Symptoms with the Functional Assessment of Cancer Therapy (FACT) Measurement System. J Pain Symptom Manage 1997, 13 (2), 63-74. https://doi.org/10.1016/s0885-3924 (96) 00274-6.
(33) EuroQol Group. EuroQol—a New Facility for the Measurement of Health-Related Quality of Life. Health Policy 1990, 16 (3), 199-208. https://doi.org/10.1016/0168-8510 (90) 90421-9.
(34) McElhone, K.; Abbott, J.; Shelmerdine, J.; Bruce, I. N.; Ahmad, Y.; Gordon, C.; Peers, K.; Isenberg, D.; Ferenkeh-Koroma, A.; Griffiths, B.; Akil, M.; Maddison, P.; Teh, L.-S. Development and Validation of a Disease-Specific Health-Related Quality of Life Measure, the LupusQol, for Adults with Systemic Lupus Erythematosus. Arthritis Rheum 2007, 57 (6), 972-979. https://doi.org/10.1002/art.22881.
(35) Jolly, M.; Pickard, A. S.; Wilke, C.; Mikolaitis, R. A.; Teh, L.-S.; McElhone, K.; Fogg, L.; Block, J. Lupus-Specific Health Outcome Measure for US Patients: The LupusQoL-US Version. Ann Rheum Dis 2010, 69 (1), 29-33. https://doi.org/10.1136/ard.2008.094763.
(36) Kroenke, K.; Spitzer, R. L. The PHQ-9: A New Depression Diagnostic and Severity Measure. Psychiatric Annals 2002, 32 (9), 509-515. https://doi.org/10.3928/0048-5713-20020901-06.
(37) Haefeli, M.; Elfering, A. Pain Assessment. Eur Spine J 2006, 15 (Suppl 1), S17-S24. https://doi.org/10.1007/s00586-005-1044-x.
(38) Touma, Z.; Urowitz, M.; Gladman, D. SLEDAI-2K for a 30-Day Window. Lupus 2010, 19 (1), 49-51. https://doi.org/10.1177/0961203309346505.
(39) Interferon-Inducible Gene Expression Kit As a Potential Diagnostic Test for Anifrolumab: Analytical Validation for Use in Clinical Trials. ACR Meeting Abstracts.
(40) Drenkard, C.; Bao, G.; Dennis, G.; Kan, H. J.; Jhingran, P. M.; Molta, C. T.; Lim, S. S. Burden of Systemic Lupus Erythematosus on Employment and Work Productivity: Data from a Large Cohort in the Southeastern United States. Arthritis Care Res (Hoboken) 2014, 66 (6), 878-887. https://doi.org/10.1002/acr.22245.
(41) Bruce, I. N.; O'Keeffe, A. G.; Farewell, V.; Hanly, J. G.; Manzi, S.; Su, L.; Gladman, D. D.; Bae, S.-C.; Sanchez-Guerrero, J.; Romero-Diaz, J.; Gordon, C.; Wallace, D. J.; Clarke, A. E.; Bernatsky, S.; Ginzler, E. M.; Isenberg, D. A.; Rahman, A.; Merrill, J. T.; Alarcón, G. S.; Fessler, B. J.; Fortin, P. R.; Petri, M.; Steinsson, K.; Dooley, M. A.; Khamashta, M. A.; Ramsey-Goldman, R.; Zoma, A. A.; Sturfelt, G. K.; Nived, O.; Aranow, C.; Mackay, M.; Ramos-Casals, M.; van Vollenhoven, R. F.; Kalunian, K. C.; Ruiz-Irastorza, G.; Lim, S.; Kamen, D. L.; Peschken, C. A.; Inanc, M.; Urowitz, M. B. Factors Associated with Damage Accrual in Patients with Systemic Lupus Erythematosus: Results from the Systemic Lupus International Collaborating Clinics (SLICC) Inception Cohort. Ann Rheum Dis 2015, 74 (9), 1706-1713. https://doi.org/10.1136/annrheumdis-2013-205171.
(42) Strand, V.; Gladman, D.; Isenberg, D.; Petri, M.; Smolen, J.; Tugwell, P. Endpoints: Consensus Recommendations from OMERACT IV. Outcome Measures in Rheumatology. Lupus 2000, 9 (5), 322-327. https://doi.org/10.1191/096120300678828424.
(43) Kaul, A.; Gordon, C.; Crow, M. K.; Touma, Z.; Urowitz, M. B.; van Vollenhoven, R.; Ruiz-Irastorza, G.; Hughes, G. Systemic Lupus Erythematosus. Nat Rev Dis Primers 2016, 2, 16039. https://doi.org/10.1038/nrdp.2016.39.
(44) Hochberg, M. C. Updating the American College of Rheumatology Revised Criteria for the Classification of Systemic Lupus Erythematosus. Arthritis Rheum 1997, 40 (9), 1725. https://doi.org/10.1002/art. 1780400928.
(45) STULL, D. E.; KERCHER, K.; KOSLOSKI, K. D. Physical Health and Long-Term Care: A Multidimensional Approach. American Behavioral Scientist 1996, 39 (3), 317-335. https://doi.org/10.1177/0002764296039003008.
(46) Yee, C.-S.; Farewell, V.; Isenberg, D. A.; Rahman, A.; Teh, L.-S.; Griffiths, B.; Bruce, I. N.; Ahmad, Y.; Prabu, A.; Akil, M.; McHugh, N.; D'Cruz, D.; Khamashta, M. A.; Maddison, P.; Gordon, C. British Isles Lupus Assessment Group 2004 Index Is Valid for Assessment of Disease Activity in Systemic Lupus Erythematosus. Arthritis Rheum 2007, 56 (12), 4113-4119. https://doi.org/10.1002/art.23130.
(47) Hurst, N. P.; Jobanputra, P.; Hunter, M.; Lambert, M.; Lochhead, A.; Brown, H. Validity of Euroqol—a Generic Health Status Instrument—in Patients with Rheumatoid Arthritis. Economic and Health Outcomes Research Group. Br J Rheumatol 1994, 33 (7), 655-662. https://doi.org/10.1093/rheumatology/33.7.655.
(48) Hurst, N. P.; Kind, P.; Ruta, D.; Hunter, M.; Stubbings, A. Measuring Health-Related Quality of Life in Rheumatoid Arthritis: Validity, Responsiveness and Reliability of EuroQol (EQ-5D). Br J Rheumatol 1997, 36 (5), 551-559. https://doi.org/10.1093/rheumatology/36.5.551.
(49) Cella, D.; Eton, D. T.; Lai, J.-S.; Peterman, A. H.; Merkel, D. E. Combining Anchor and Distribution-Based Methods to Derive Minimal Clinically Important Differences on the Functional Assessment of Cancer Therapy (FACT) Anemia and Fatigue Scales. J Pain Symptom Manage 2002, 24 (6), 547-561. https://doi.org/10.1016/s0885-3924 (02) 00529-8.
(50) Bentler, P. M. Comparative Fit Indexes in Structural Models. Psychol Bull 1990, 107 (2), 238-246. https://doi.org/10.1037/0033-2909.107.2.238.
(51) Steiger, J. H. Structural Model Evaluation and Modification: An Interval Estimation Approach. Multivariate Behavioral Research 1990, 25 (2), 173-180. https://doi.org/10.1207/s15327906mbr2502_4.
(52) Talk: Systemic Lupus Erythematosus Responder Index Assessment of Responders in EMBLEM, a Phase IIb Study in Patients with Moderate to Severe Systemic Lupus Erythematosus (2011 ACR/ARHP Annual Scientific Meeting) https://acr.confex.com/acr/2011/webprogram/Paper22284.html (accessed 2021 Aug. 25).
(53) van Vollenhoven, R. F.; Hahn, B. H.; Tsokos, G. C.; Wagner, C. L.; Lipsky, P.; Touma, Z.; Werth, V. P.; Gordon, R. M.; Zhou, B.; Hsu, B.; Chevrier, M.; Triebel, M.; Jordan, J. L.; Rose, S. Efficacy and Safety of Ustekinumab, an IL-12 and IL-23 Inhibitor, in Patients with Active Systemic Lupus Erythematosus: Results of a Multicentre, Double-Blind, Phase 2, Randomised, Controlled Study. Lancet 2018, 392 (10155), 1330-1339. https://doi.org/10.1016/S0140-6736 (18) 32167-6.
(54) Thanou, A.; Chakravarty, E.; James, J. A.; Merrill, J. T. Which Outcome Measures in SLE Clinical Trials Best Reflect Medical Judgment? Lupus Sci Med 2014, 1 (1), e000005. https://doi.org/10.1136/lupus-2013-000005.
(55) Pike, M. C.; Kelley, L. Data Quality Challenges in Systemic Lupus Erythematosus Trials: How Can This Be Optimized? Curr Rheumatol Rep 2012, 14 (4), 324-333. https://doi.org/10.1007/s11926-012-0261-7.

1. (canceled)

2. (canceled)

3. (canceled)

4. (canceled)

5. (canceled)

6. (canceled)

7. (canceled)

8. (canceled)

9. (canceled)

10. (canceled)

11. A method of treating pain in a subject in need thereof, the method comprising administering to the subject a therapeutically effective amount of anifrolumab, wherein the subject has systemic lupus erythematosus (SLE).

12. The method of claim 11, wherein the pain is bodily pain.

13. The method of claim 11, wherein the pain is abdominal pain, headache, and/or pain associated with the Raynaud's phenomenon.

14. The method of claim 11, wherein the method reduces fatigue in the subject.

15. The method of claim 11, wherein the method improves the subject's mood.

16. The method of claim 11, wherein the method improves the subject's physical functioning.

17. The method of claim 11, wherein the method reduces SLE disease activity in the subject.

18. The method of claim 17, wherein the reduction in SLE disease activity comprises a BILCA and/or SRI(4) response.

19. (canceled)

20. The method of claim 11, the method comprising steroid sparing in the subject, wherein the dose of the steroid administered to the subject is tapered from a pre-sparing dose at baseline to a post-sparing dose, wherein the pre-sparing dose is 20 mg/day prednisone or prednisone equivalent dose.

21. (canceled)

22. (canceled)

23. (canceled)

24. (canceled)

25. (canceled)

26. (canceled)

27. The method of claim 11, wherein the subject has moderate to severe SLE.

28. The method of claim 11, wherein the method has been demonstrated in a phase III clinical trial.

29. (canceled)

30. (canceled)

31. (canceled)

32. (canceled)

33. (canceled)

34. The method of claim 11, wherein the method comprises administering a fixed dose of anifrolumab.

35. The method of claim 34, wherein the method comprises administering about 300 mg to about 1000 mg of anifrolumab.

36. The method of claim 35, comprising administering about 300 mg anifrolumab.

37. The method of claim 34, comprising administering anifrolumab at a dose of about 300 to about 1000 mg every four weeks (Q4W).

38. The method of claim 11, wherein anifrolumab is administered intravenously.

39. The method of claim 34, comprising administering anifrolumab to the patient at a dose of greater than 105 mg and less than 150 mg.

40. The method of claim 39, comprising administering anifrolumab to the patient at a dose of about 120 mg per week.

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. The method of claim 39, wherein anifrolumab is administered subcutaneously.
